(19) United States
(12) Patent Application Publication
(10) Pub. No.: US 2021/0079054 A1
(43) Pub. Date: Mar. 18, 2021

(54) PEPTIDE IMMUNOGENS OF IL-31 AND FORMULATIONS THEREOF FOR THE TREATMENT AND/OR PREVENTION OF ATOPIC DERMATITIS

(71) Applicant: UBI US Holdings, LLC., Hauppauge, NY (US)

(72) Inventor: Chang Yi WANG, Cold Spring Harbor, NY (US)

(21) Appl. No.: 16/771,948

(22) PCT Filed: Dec. 11, 2018
(86) PCT No.: PCT/US18/65025

§ 371 Date: Jun. 11, 2020

Related U.S. Application Data
(60) Provisional application No. 62/597,130, filed on Dec. 11, 2017.

(51) Int. Cl. C07K 14/54; C07K 16/24; A61K 39/00; A61K 39/35

(52) U.S. Cl. CPC C07K 14/54; C07K 16/244; A61K 38/00; A61K 39/35; A61K 39/0008

(57) ABSTRACT

The present disclosure is directed to individual peptide immunogen constructs targeting portions of the Interleukin-31 (IL-31) protein for the treatment and/or prevention of a pruritic condition and/or an allergic condition, such as atopic dermatitis. The present disclosure is also directed to compositions containing the peptide immunogen constructs, methods of making and using the peptide immunogen constructs, and antibodies produced by the peptide immunogen constructs.

The present application is a PCT International Application that claims the benefit of U.S. Provisional Application Ser. No. 62/597,130, filed Dec. 11, 2017, which is incorporated herein by reference in its entirety.

### FIELD OF THE INVENTION

This disclosure relates to peptide immunogen constructs targeting Interleukin-31 (IL-31) and formulations thereof as a pharmaceutical composition for the treatment and/or prevention of atopic dermatitis.

### BACKGROUND OF THE INVENTION

Atopic dermatitis (AD) has been defined by the American College of Veterinary Dermatology task force as “a genetically-predisposed inflammatory and pruritic allergic skin disease with characteristic clinical features” (Olivry 2001). The task force also recognized that the disease in canines has been associated with allergen-specific IgE (Olivry 2001: Marsella & Olivry 2003). Severe pruritus, along with secondary alopecia and erythema, are the most noticeable and concerning symptoms to pet owners (U.S. Pat. No. 8,790,651).

The prevalence of atopic dermatitis is not known with precision due to poor and inconsistent epidemiological data, but is estimated to be 10% of the total canine population (Marsella & Olivry 2003. Scott 2002; Hillier 2001). Globally, about 4.5 million dogs are affected with this chronic and lifelong condition. Incidence appears to be increasing. Breed and sex predilections have been suspected, but may vary greatly depending on geographical region (Hillier 2001; Picco 2008).

The potential factors involved in allergic dermatitis are numerous and poorly understood. Components in food may trigger atopic dermatitis (Picco, 2008), as well as environmental allergens such as fleas, dust mites, ragweed, plant extracts, etc. Genetic factors also play an important role. Although there is no confirmed breed predilection, some mode of inheritance is thought to increase predisposition to atopic dermatitis (Sousa & Marsella 2001; Schwartzman 1971).

Interleukin-31 (IL-31) is a cytokine that was cloned in 2004. It is mainly produced by activated T helper (Th)2 cells (Dillon 2004), but is also produced in mast cells and macrophages. IL-31 binds a co-receptor composed of IL-31 receptor A (IL-31RA) and the oncostatin M receptor (OSMR) (Dillon 2004 and Bilsborough 2006). Receptor activation results in phosphorylation of STAT through JAK receptor(s). Expression of the co-receptor has been shown in macrophages, keratinocytes and in dorsal root ganglia. Recently, it has been found that IL-31 is involved in dermatitis, pruritic skin lesions, allergy and airway hypersensitivity.

Stimulation of T cells with anti-CD3 and anti-CD28 antibodies immediately upregulates IL-31 mRNA expression (Dillon 2004). Microarray analysis has shown that IL-31 induces certain chemotactic genes, such as CXCL1, CLL17 (thymus and activation-regulated chemokine (TARC)), CCL19 (macrophage inflammatory protein (MIP)3β), CCL22 (monocyte-derived chemokine (MDC), CCL23 (MIP3), and CCL4 (MIPβ)) (Dillon 2004).

Transgenic mice that over-express IL-31 show skin inflammation, pruritis, severe dermatitis, and alopecia (Dillon 2004). Subcutaneous injection of IL-31 into mice triggers infiltration by the inflammatory cells, neutrophils, eosinophils, lymphocytes, and macrophages, and results in epidermal thickening and dermal acanthosis. In NC/Nga mice, with atopic dermatitis (AD) due to natural causes, IL-31 is overexpressed in skin lesions and correlates with pruritus (Takaoka 2005: Takaoka 2006). Also, in murine models, IL-31 has been shown to induce rapid onset pruritus (Raap 2008).

Further studies have indicated that IL-31 is associated with atopic-dermatitis-induced skin inflammation and pruritus in humans. In human AD patients, the expression of IL-31 mRNA is considerably higher in skin lesions than in non-lesional skin, and the expression in non-lesional skin is greater than that in normal skin from healthy patients (Sonkoly 2006). Another study has reported that CD45RO+(memory) cutaneous lymphocyte antigen (CLA)-positive T cells in the skin of AD patients express IL-31 mRNA and protein (Bilsborough 2006). It has also been reported that IL-31 mRNA overexpression in the skin of patients or allergic contact dermatitis is correlated with IL-4 and IL-13 mRNA expression, but not with interferon (IFN)-γ mRNA expression (Neis 2006). Furthermore, IL-31 serum levels have been shown to be elevated in human patients with chronic spontaneous urticaria and even more so in patients with AD (Raap 2010). Also, a correlation of the severity of AD with serum IL-31 levels has been observed in humans (Rapp 2008). IL-31 secretion has also been shown to be enhanced in mast cells following IgE cross-linking and as a response to Staphylococcal superantigen in atopic individuals. In addition, IL-31 has been shown to stimulate the production of several pro-inflammatory mediators including IL-6, IL-8, CXCL1, CC17 and multiple metalloproteinases in human colonic myofibroblasts (Yagi 2007).

Type I hypersensitivity against environmental allergens is considered to be the main mechanism of canine AD, and the levels of Th2-mediated cytokines, such as IL-4 are increased in the skin lesions of dogs with AD (Nuttall 2002). Moreover, infiltration by inflammatory cells, lymphocytes and neutrophils, is an important mechanism underlying the aggravation of the skin lesions; the overexpression of chemotactic genes such as CCL17/TARC, CCR4, and CCL28/mucosae-associated epithelial chemokine (MEC) contributes to the aggravation of skin lesions in the dogs with AD (Maeda 2005; Maeda 2002; and Maeda 2008).

Recent evidence has suggested that IL-31 might be involved in promoting allergic inflammation and an airway epithelial response characteristic of allergic asthma (Chattopadhyay 2007; and Wai 2007).

These observations support the hypothesis that IL-31 plays a significant role in both pruritic and allergic conditions. It would be desirable to provide a peptide immunogen capable of eliciting antibodies against IL-31 useful for treating a pruritic condition and/or an allergic condition, such as atopic dermatitis in dogs, cats, and/or humans.

### REFERENCES

1. BAMMERT, et al., U.S. Pat. No. 8,790,651 (issued Jul. 29, 2014)
2. BILSBOROUGH, et al., J Allergy Clin Immunol., 117(2):418-25 (2006)
3. CHATTOPADHYAY, et al., J Biol Chem, 282:3014-26 (2007)
4. LENG, et al., U.S. Pat. No. 8,426,163 (issued Apr. 23, 2013)
5. DILLON, et al., Nat Immunol, 5:752-60 (2004)
6. HILLIER, Veterinary Immunology and Immunopathology, 81: 147-151 (2001)
7. MAEDA, et al., Vet. Immunol. Immunopathol., 90, 145-154; (2002)
8. MAEDA, et al., Vet. Immunol. Immunopathol., 103, 83-92; (2003)
9. MAEDA, et al., J. Vet. Med. Sci., 70, 51-55 (2008)
10. MARSELLA & OLIVRY, Clinics in Dermatology, 21: 122-133 (2003)
11. NEIS, et al., J. Allergy Clin. Immunol., 118, 930-937 (2006)
12. NUTTALL, et al., Vet. Immunol. Immunopathol., 87, 379-384 (2002)
13. OLIVRY, et al., Veterinary Immunology and Immunopathology, 81: 143-146 (2001)
14. PICCO, et al., Vet Dermatol., 19: 150-155 (2008)
15. RAAP, et al., J Allergy Clin Immunol., 122(2):421-3 (2008)
16. RAAP, et al., Exp Dermatol., 19(5):464-6 (2010)
17. SCHWARTZMAN, et al., Clin. Exp. Immunol., 9: 549-569 (2971)
18. SCOTT, et al., Canadian Veterinary Journal, 43: 601-603 (2002)
19. SONKOLY, et al., J Allergy Clin Immunol., 117:411-7 (2006)
20. SOUSA, et al., Veterinary Immunology and Immunopathology, 81:153-157 (2001)
21. TAKAOKA, et al., Eur J. Pharmacol., 516, 180-181 (2005)
22. TAKAOKA, et al., Exp. Dermatol., 15, 161-167 (2006)
23. WAI, et al., Immunology, 122, 532-541 (2007)
24. WALKER, et al., U.S. Pat. No. 6,361,966 (issued Mar. 26, 2002)
25. YAGI, et al., International Journal of Molecular Medicine, 19(6): 941-946 (2007)
26. “Atopic Dermatitis”, Wikipedia, The Free Encyclopedia, website address: en.wikipedia.org/wiki/Atopic_dermatitis (accessed Dec. 8, 2017).
27. “Interleukin 31”, Wikipedia, The Free Encyclopedia, website address: en.wikipedia.org/wiki/Interleukin_31 (accessed Dec. 8, 2017).

### SUMMARY OF THE INVENTION

The present disclosure is directed to individual peptide immunogen constructs targeting portions of the Interleukin-31 (IL-31) protein for the treatment and/or prevention of a pruritic condition and/or an allergic condition, such as atopic dermatitis. The present disclosure is also directed to compositions containing the peptide immunogen constructs, methods of making and using the peptide immunogen constructs, and antibodies produced by the peptide immunogen constructs.

The disclosed peptide immunogen constructs contain about 25 or more amino acids. The peptide immunogen constructs contain a B cell epitope from portions of the canine IL-31 protein (GenBank: BAH97742.1). The full-length amino acid sequence of the canine and human IL-31 protein is shown as SEQ ID NO: 1 and SEQ ID NO: 2, respectively, in Table 1. The B cell epitope can be linked to a heterologous T helper cell (Th) epitope derived from pathogen proteins through an optional heterologous spacer. The disclosed peptide immunogen constructs stimulate the generation of highly specific antibodies directed against IL-31. The disclosed peptide immunogen constructs can be used as an immunotherapy for animals suffering from a pruritic condition and/or an allergic condition, such as atopic dermatitis.

The B cell epitope portion of the peptide immunogen constructs have amino acid sequences from the full-length canine IL-31 protein (SEQ ID NO: 1) or the full-length human IL-31 protein (SEQ ID NO: 2). In some embodiments, the B cell epitope has a sequence containing any of SEQ ID NOs: 1 to 13 and 93 to 98, as shown in Table 1.

The peptide immunogen constructs of the present disclosure can contain a heterologous Th epitope amino acid sequence derived from a pathogenic protein (e.g., SEQ ID NOs: 14 to 42) as shown in Table 2. In certain embodiments, the heterologous Th epitope is derived from natural pathogens, such as Diphtheria Toxin (SEQ ID NO: 18), Plasmodium falciparum (SEQ ID NO: 19), Cholera Toxin (SEQ ID NO: 21). In other embodiments, the heterologous Th epitope is an idealized artificial Th epitope derived from Measles Virus Fusion protein (MVF 1 to 5) or Hepatitis B Surface Antigen (HBsAg 1 to 3) in the form of either single sequence or combinatorial sequences (e.g., SEQ ID NOs: 25, 24, and 26).

In some embodiments, the peptide immunogen constructs contain a B cell epitope from IL-31 linked to a heterologous T helper cell (Th) epitope through an optional heterologous spacer. In certain embodiments, the peptide immunogen constructs contain a B cell antigenic site having the amino acid sequence from IL-31 (e.g., SEQ ID NOs: 1 to 13 and 93 to 98) linked to a heterologous Th epitope derived from a pathogenic protein (e.g., SEQ ID NOs: 14 to 42) through an optional heterologous spacer. In some embodiments, the optional heterologous spacer is a molecule or chemical structure capable of linking two amino acids and/or peptides together. In certain embodiments, the spacer is a naturally occurring amino acid, a non-naturally occurring amino acid, or a combination thereof. In specific embodiments, the peptide immunogen constructs have the amino acid sequence of SEQ ID NOs: 43 to 90 and 99 to 105 shown in Table 3.

The present disclosure is also directed to compositions containing an IL-31 peptide immunogen construct. In some embodiments, the disclosed compositions contain more than one IL-31 peptide immunogen constructs. In certain embodiments, the compositions contain a mixture of IL-31 peptide immunogen constructs (e.g., any combination of SEQ ID NOs: 43-90 and 99 to 105) to cover a broad genetic background in subjects. Compositions containing a mixture of peptide immunogen constructs can lead to a higher percentage in responder rate upon immunization for the treatment of a pruritic condition and/or an allergic condition, such as atopic dermatitis, compared to compositions containing only a single peptide immunogen construct.

The present disclosure is also directed to pharmaceutical compositions for the treatment and/or prevention of a pruritic condition and/or an allergic condition, such as atopic dermatitis. In some embodiments, the pharmaceutical compositions contain the disclosed peptide immunogen constructs in the form of a stabilized immunostimulatory complex formed through electrostatic associations by mixing a CpG oligomer with a composition containing a peptide immunogen complex. Such stabilized immunostimulatory complexes are able to further enhance the immunogenicity of the peptide immunogen constructs. In some embodiments, the pharmaceutical compositions contain adjuvants such as mineral salts, including alum gel (ALHYDROGEL), aluminum phosphate (ADJUPHOS), or water-in-oil emulsions including MONTANIDE ISA 50V2, ISA 51, or ISA 720.

The present disclosure is also directed to antibodies directed against the disclosed IL-31 peptide immunogen constructs. In particular, the peptide immunogen constructs of the present disclosure are able to stimulate the generation of highly specific antibodies that are cross-reactive with the IL-31 amino acid sequences (SEQ ID NOs: 1-13) when administered to a subject. The highly specific antibodies produced by the peptide immunogen constructs are cross reactive with recombinant IL-31-containing proteins. The disclosed antibodies bind with high specificity to IL-31 without much, if any, directed to the heterologous Th epitopes employed for immunogenicity enhancement, which is in sharp contrast to the conventional protein or other biological carriers used for such peptide antigenicity enhancement.

The present disclosure also includes methods for treating and/or preventing a pruritic condition and/or an allergic condition, such as atopic dermatitis, using the disclosed peptide immunogen constructs and/or antibodies directed against the peptide immunogen constructs. In some embodiments, the methods for treating and/or preventing a pruritic condition and/or an allergic condition, such as atopic dermatitis, include administering to a host a composition containing a disclosed peptide immunogen construct. In certain embodiments, the compositions utilized in the methods contain a disclosed peptide immunogen construct in the form of a stable immunostimulatory complex with negatively charged oligonucleotides, such as CpG oligomers, through electrostatic association, which complexes are further supplemented, optionally, with mineral salts or oil as adjuvant, for administration to subjects with a pruritic condition and/or an allergic condition, such as atopic dermatitis. The disclosed methods also include dosing regimens, dosage forms, and routes for administering the peptide immunogen constructs to a host at risk for, or with, a pruritic condition and/or an allergic condition, such as atopic dermatitis.

### BRIEF DESCRIPTION OF THE DRAWINGS

FIGS. 1A to 1B—Amino acid sequence of canine IL-31 protein (SEQ ID NO: 1) with structural and functional features highlighted (FIG. 1A) and sequence alignment and localization of antigenic sites used in the present disclosure relative to the full-length canine IL-31 protein (FIG. 1B).

FIG. 2—Graphs illustrating the anti-IL-31 polyclonal antibody titers (Log₁₀) with recombinant canine IL-31 protein after immunization of guinea pigs with various formulations containing the IL-31 peptide immunogens in adjuvant ISA50V2. Samples were analyzed at 3, 6, 9, 12, and 15 weeks post injection (wpi).

FIGS. 3A to 3D—Plasmid map of canine IL-31 with a His-tag at the C-terminus for expression in mammalian cells (Expi 293F) (FIG. 3A) and a Westem blot probed with an anti-His-tag antibody from cell lysate and culture medium (FIG. 3B). Coomassie blue staining of His-tagged IL-31 protein expression and purification run on a 12% Bis Tris SDS PAGE (FIG. 3C). Westem blot of the SDS-PAGE gel shown in FIG. 3C, probed with an anti-His-tag antibody (FIG. 3D).

FIGS. 4A to 4B—schematic illustrating an in vitro IL-31 induced signal transduction functional inhibition assay using IL-31 antibodies (FIG. 4A). An enlarged view of the assay steps is shown in FIG. 4B.

FIGS. 5A and 5B—Graphs showing the establishment of an in vitro functional assay measuring the phosphorylation level of STAT3 in canine DH82 monocytes induced by canine IL-31 produced from expi293 cells. FIG. 5A shows pSTAT3 was induced by canine IL-31 in a dose-dependent manner. FIG. 5B shows pSTAT3 was induced by canine IL-31 in a time-dependent manner.

FIGS. 6A to 6B—Graphs showing the inhibition of canine IL-31 (1 μg/mL) induced pSTAT3 signaling in canine DH82 monocytes by anti-IL-31 antibodies derived from IL-31 peptide immunogen constructs (SEQ ID NOs: 43, 47, 51, 55, and 59) shown at 12 weeks post initial immunization (wpi) (FIG. 6A) and 15 wpi (FIG. 6B).

FIG. 7—Graph showing the inhibition of canine IL-31 (1 μg/mL) induced pSTAT3 signaling in canine DH82 monocytes by anti-IL-31 antibodies derived from IL-31 peptide immunogen constructs p4751 kb (SEQ ID NO: 43) and p4752 (SEQ ID NO: 47) at 15 weeks post injection (wpi). The bottom panel reports the inhibition (IC₅₀) to recombinant canine IL-31 protein induced pSTAT signaling.

FIG. 8—Graphs illustrating the anti-IL-31 polyclonal antibody titers (Log₁₀) with recombinant canine IL-31 protein after immunization of guinea pigs with formulations containing the IL-31 peptide immunogens (SEQ ID NOs: 63, 68, 71, 76, 80, 84) in adjuvant ISA50V2. Samples were analyzed at 3, 6, 9, 12, and 15 weeks post initial immunization (wpi).

FIGS. 9A to 9B—Graphs showing the percent phosphorylation (FIG. 9A) and percent inhibition (FIG. 9B) of canine IL-31 (1 μg/mL) induced pSTAT3 signaling in canine DH82 monocytes by anti-IL-31 antibodies derived from the disclosed IL-31 peptide immunogen constructs (SEQ ID NOs: 63, 68, 71, 76, 80 and 84) shown at 6 and 12 weeks post initial immunization (wpi).

FIGS. 10A to 10B—Graphs showing the percent phosphorylation (FIG. 10A) and percent inhibition (FIG. 10B) of canine IL-31 (1 μg/mL) induced pSTAT3 signaling in canine DH82 monocytes by anti-IL-31 antibodies derived from the disclosed IL-31 peptide immunogen constructs (SEQ ID Nos: 63, 68, 71 and 64) shown at 9 and 12 weeks post initial immunization (wpi).

FIGS. 11A to 11B—Bar graphs showing the percent phosphorylation (FIG. 11A) and percent inhibition (FIG. 11B) of canine IL-31 (1 μg/mL) induced pSTAT3 signaling in canine DH82 monocytes by anti-IL-31 antibodies derived from IL-31 peptide immunogen constructs p4854 kb (SEQ ID NO: 63) and p4859 (SEQ ID NO: 84) at 6, 9, and 12 weeks post initial immunization (wpi).

FIG. 12—Curve graphs showing the percent phosphorylation of canine IL-31 (1 μg/mL) induced pSTAT3 signaling in canine DH82 monocytes by anti-IL-31 antibodies derived from IL-31 peptide immunogen constructs p4854 kb (SEQ ID NO: 63) and p4859 (SEQ ID NO: 84) at 6, 9, and 12 weeks post initial immunization (wpi). The bottom panel reports the inhibition (IC₅₀) to recombinant canine IL-31 protein induced pSTAT signaling.

FIG. 13—Immunization protocol of beagle dogs using canine IL-31 peptide immunogen construct (SEQ ID NO: 84).

FIG. 14—Assessment of immunogenicity of human IL-31 peptide immunogen construct (SEQ ID NO: 84) (Log₁₀) to the B cell epitope peptide (2^{nd} Ab: Goat anti-dog IgG HRP) in beagle dogs.

FIG. 15—Dog Serum antibody titers (Log 10) to rcIL-31 protein using IL-31 peptide immunogen construct (SEQ ID NO:84) (2^{nd} Ab: rabbit anti-dog IgG HRP) at 21 and 41 days post initial immunization (DPI).

FIG. 16—Dog Serum antibody titers (Log 10) to rcIL-31 Protein using IL-31 peptide immunogen construct (SEQ ID NO:84) (2^{nd} Ab: protein A/G-HRP) at 21 and 41 days post initial immunization (DPI).

FIGS. 17A to 17B—Graphs showing the percent phosphorylation (FIG. 17A) and percent inhibition (FIG. 17B) of canine IL-31 induced pSTAT signaling in canine DH82 monocytes by purified IgGs from dog immune sera (representative samples 1 to 10).

FIGS. 18A to 183—Graphs showing the percent phosphorylation (FIG. 18A) and percent inhibition (FIG. 18B) of canine IL-31 induced pSTAT signaling in canine DH82 monocytes by Cytopoint (anti-IL-31 mAb)—IC₅₀: 3.21 μg/mL.

FIGS. 19A to 19C—Schematic showing the immunization and bleeding schedule of guinea pigs immunized with various IL-31 peptide immunogen constructs (SEQ ID NOs: 83 and 85) is provided in FIG. 19A. Graphs showing the percent phosphorylation (FIG. 19B) and percent inhibition (FIG. 19C) of canine IL-31 induced pSTAT signaling in canine DH82 monocytes by purified IgGs from guinea pig immune sera of 6 and 15 wpi, respectively.

FIGS. 20A to 20B—Graph illustrating the immunogenicity of various human IL-31 peptide immunogen constructs (SEQ ID NOs: 87, 99, 100, and 101) in guinea pigs (FIG. 20A) together with a summary of the Log EC₅₀ for each construct (FIG. 20B).

FIG. 21—Graph illustrating the inhibition of IL-31 and IL-31Rα interaction by IL-31 reactive polyclonal antibodies from guinea pig immune sera against IL-31 peptide immunogen constructs (SEQ ID NOs: 87, 99, 100, and 101) together with a summary of the IC₅₀ for each construct.

FIG. 22—Suppression of IL-31 induced STAT3 phosphorylation on U87MG cells by IL-31 reactive polyclonal antibodies from guinea pig immune sera against IL-31 peptide immunogen constructs (SEQ ID NOs: 87 and 101).

FIG. 23—Suppression of IL-31 induced IL-20 expression on HaCaT cells by IL-31 reactive polyclonal antibodies from guinea pig immune sera against IL-31 peptide immunogen constructs (SEQ ID NOs: 87 and 101).

FIGS. 24A to 24B—Graph illustrating the immunogenicity of a representative human IL-31 peptide construct (SEQ ID NO: 101) in various multi-component formulations (FIG. 24A) together with a summary of the Log EC₅₀ for each formulation (FIG. 24B).

FIGS. 25A to 25B—Graph showing inhibition of IL-31 and IL-31a interaction by IL-31 reactive polyclonal antibodies from guinea pig immune sera against human IL-31 peptide immunogen construct (SEQ ID NO: 101) in various formulations (FIG. 25A) together with a summary of the IC₅₀ for each formulation (FIG. 25B).

FIG. 26—Suppression of IL-31 induced IL-20 expression on HaCaT cells by IL-31 reactive polyclonal antibodies from guinea pig immune sera against IL-31 peptide immunogen construct (SEQ ID NO:101) in various formulations.

### DETAILED DESCRIPTION OF THE INVENTION

The present disclosure is directed to individual peptide immunogen constructs targeting portions of the Interleukin-31 (IL-31) protein for the treatment and/or prevention of a pruritic condition and/or an allergic condition, such as atopic dermatitis. The present disclosure is also directed to compositions containing the peptide immunogen constructs, methods of making and using the peptide immunogen constructs, and antibodies produced by the peptide immunogen constructs.

The disclosed peptide immunogen constructs contain about 25 or more amino acids. The peptide immunogen constructs contain a B cell epitope from portions of the canine IL-31 protein (GenBank: BAH97742.1). The full-length amino acid sequence of the canine and human IL-31 protein is shown as SEQ ID NO: 1 and SEQ ID NO: 2, respectively, in Table 1. The B cell epitope can be linked to a heterologous T helper cell (Th) epitope derived from pathogen proteins through an optional heterologous spacer. The disclosed peptide immunogen constructs stimulate the generation of highly specific antibodies directed against IL-31. The disclosed peptide immunogen constructs can be used as an immunotherapy for animals suffering from a pruritic condition and/or an allergic condition, such as atopic dermatitis.

The B cell epitope portion of the peptide immunogen constructs have amino acid sequences from the full-length canine IL-31 protein (SEQ ID NO: 1) or the full-length human IL-31 protein (SEQ ID NO: 2). In some embodiments, the B cell epitope has a sequence containing any of SEQ ID NOs: 1 to 13 and 93 to 98 as shown in Table 1.

The peptide immunogen constructs of the present disclosure can contain a heterologous Th epitope amino acid sequence derived from a pathogenic protein (e.g., SEQ ID NOs: 14 to 42) as shown in Table 2. In certain embodiments, the heterologous Th epitope is derived from natural pathogens, such as Diphtheria Toxin (SEQ ID NO: 18), Plasmodium falciparum (SEQ ID NO: 19), Cholera Toxin (SEQ ID NO: 21). In other embodiments, the heterologous Th epitope is an idealized artificial Th epitope derived from Measles Virus Fusion protein (MVF 1 to 5) or Hepatitis B Surface Antigen (HBsAg 1 to 3) in the form of either single sequence or combinatorial sequences (e.g., SEQ ID NOs: 25, 24, and 26).

In some embodiments, the peptide immunogen constructs contain a B cell epitope from IL-31 linked to a heterologous T helper cell (Th) epitope through an optional heterologous spacer. In certain embodiments, the peptide immunogen constructs contain a B cell antigenic site having the amino acid sequence from IL-31 (e.g., SEQ ID NOs: 1 to 13 and 93 to 98) linked to a heterologous Th epitope derived from a pathogenic protein (e.g., SEQ ID NOs: 14 to 42) through an optional heterologous spacer. In some embodiments, the optional heterologous spacer is a molecule or chemical structure capable of linking two amino acids and/or peptides together. In certain embodiments, the spacer is a naturally occurring amino acid, a non-naturally occurring amino acid, or a combination thereof. In specific embodiments, the peptide immunogen constructs have the amino acid sequence of SEQ ID NOs: 43 to 90 and 99 to 105 shown in Table 3.

The present disclosure is also directed to compositions containing an IL-31 peptide immunogen construct. In some embodiments, the disclosed compositions contain more than one IL-31 peptide immunogen constructs. In certain embodiments, the compositions contain a mixture of IL-31 peptide immunogen constructs (e.g., any combination of SEQ ID NOs: 43-90 and 99 to 105) to cover a broad genetic background in subjects. Compositions containing a mixture of peptide immunogen constructs can lead to a higher percentage in responder rate upon immunization for the treatment of a pruritic condition and/or an allergic condition, such as atopic dermatitis, compared to compositions containing only a single peptide immunogen construct.

The present disclosure is also directed to pharmaceutical compositions for the treatment and/or prevention of a pruritic condition and/or an allergic condition, such as atopic dermatitis. In some embodiments, the pharmaceutical compositions contain the disclosed peptide immunogen constructs in the form of a stabilized immunostimulatory complex formed through electrostatic associations by mixing a CpG oligomer with a composition containing a peptide immunogen complex. Such stabilized immunostimulatory complexes are able to further enhance the immunogenicity of the peptide immunogen constructs. In some embodiments, the pharmaceutical compositions contain adjuvants such as mineral salts, including alum gel (ALHYDROGEL), aluminum phosphate (ADJUPHOS), or water-in-oil emulsions including MONTANIDE ISA 50V2, ISA 51, or ISA 720.

The present disclosure is also directed to antibodies directed against the disclosed IL-31 peptide immunogen constructs. In particular, the peptide immunogen constructs of the present disclosure are able to stimulate the generation of highly specific antibodies that are cross-reactive with the IL-31 amino acid sequences (SEQ ID NOs: 1-13 and 93 to 98) when administered to a subject. The highly specific antibodies produced by the peptide immunogen constructs are cross reactive with recombinant IL-31-containing proteins. The disclosed antibodies bind with high specificity to IL-31 without much, if any, directed to the heterologous Th epitopes employed for immunogenicity enhancement, which is in sharp contrast to the conventional protein or other biological carriers used for such peptide antigenicity enhancement.

The present disclosure also includes methods for treating and/or preventing a pruritic condition and/or an allergic condition, such as atopic dermatitis, using the disclosed peptide immunogen constructs and/or antibodies directed against the peptide immunogen constructs. In some embodiments, the methods for treating and/or preventing a pruritic condition and/or an allergic condition, such as atopic dermatitis, include administering to a host a composition containing a disclosed peptide immunogen construct. In certain embodiments, the compositions utilized in the methods contain a disclosed peptide immunogen construct in the form of a stable immunostimulatory complex with negatively charged oligonucleotides, such as CpG oligomers, through electrostatic association, which complexes are further supplemented, optionally, with mineral salts or oil as adjuvant, for administration to subjects with a pruritic condition and/or an allergic condition, such as atopic dermatitis. The disclosed methods also include dosing regimens, dosage forms, and routes for administering the peptide immunogen constructs to a host at risk for, or with, a pruritic condition and/or an allergic condition, such as atopic dermatitis.

The section headings used herein are for organizational purposes only and are not to be construed as limiting the subject matter described. All references or portions of references cited in this application are expressly incorporated by reference herein in their entirety for any purpose.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control.

In addition, the materials, methods, and examples are illustrative only and not intended to be limiting.

### IL-31 Peptide Immunogen Construct

The present disclosure provides peptide immunogen constructs containing a B cell epitope with an amino acid sequence from IL-31 covalently linked to a heterologous T helper cell (Th) epitope directly or through an optional heterologous spacer.

The phrase “IL-31 peptide immunogen construct” or “peptide immunogen construct”, as used herein, refers to a peptide containing (a) a B cell epitope having about 15 or more amino acid residues from the full-length sequence of canine IL-31 (SEQ ID NO: 1) or human IL-31 (SEQ ID NO: 2); (b) a heterologous Th epitope; and (c) an optional heterologous spacer.

In certain embodiments, the IL-31 peptide immunogen construct can be represented by the formulae:

(Th)ₘ-(A)ₙ-(IL-31 fragment)-X

or

(IL-31 fragment)-(A)ₙ-(Th)ₘ-X

wherein

Th is a heterologous T helper epitope;

A is a heterologous spacer;

(IL-31 fragment) is a B cell epitope having about 15 to about 75 amino acid residues from SEQ ID NO: 1 or SEQ ID NO: 2:

X is an α-COOH or α-CONH₂ of an amino acid;

m is from 1 to about 4; and

n is from 0 to about 10.

The various components of the disclosed IL-31 peptide immunogen construct are described below.

a. IL-31 Fragments

The disclosed peptide immunogen constructs contain about 25 or more total amino acids, with about 15 amino acids from the IL-31 protein. The peptide immunogen constructs contain a B cell epitope from canine IL-31 protein (GenBank: BAH97742.1) having the amino acid sequence of SEQ ID NO: 1 shown in Table 1 or human IL-31 protein (GenBank: AAS86448.1) having the amino acid sequence of SEQ ID NO: 2 shown in Table 1. The B cell epitope can be linked to a heterologous T helper cell (Th) epitope derived from pathogen proteins through an optional heterologous spacer. The disclosed peptide immunogen constructs stimulate the generation of highly specific antibodies directed against IL-31. The disclosed peptide immunogen constructs can be used as an immunotherapy for subjects having a pruritic condition and/or an allergic condition, such as atopic dermatitis.

In some embodiments, the B cell epitope has a sequence containing any of SEQ ID NOs: 1-13 and 93 to 98 as shown in Table 1. The IL-31 fragments shown in Table 1 are exemplary and the present disclosure includes any other fragment of the full-length canine IL-31 protein or human IL-31 protein of SEQ ID NOs: 1 and 2, respectively.

b. Heterologous T Helper Cell Epitopes (Th Epitopes)

The present disclosure provides peptide immunogen constructs containing a B cell epitope from IL-31 covalently linked to a heterologous T helper cell (Th) epitope directly or through an optional heterologous spacer.

The heterologous Th epitope in the IL-31 peptide immunogen construct enhances the immunogenicity of the IL-31 fragments, which facilitates the production of specific high titer antibodies directed against the optimized target B cell epitope (i.e., the IL-31 fragment) through rational design.

The term “heterologous”, as used herein, refers to an amino acid sequence that is derived from an amino acid sequence that is not part of, or homologous with, the wild-type sequence of IL-31. Thus, a heterologous Th epitope is a Th epitope derived from an amino acid sequence that is not naturally found in IL-31 (i.e., the Th epitope is not autologous to IL-31). Since the Th epitope is heterologous to IL-31, the natural amino acid sequence of IL-31 is not extended in either the N-terminal or C-terminal directions when the heterologous Th epitope is covalently linked to the IL-31 fragment.

The heterologous Th epitope of the present disclosure can be any Th epitope that does not have an amino acid sequence naturally found in IL-31. The Th epitope can have an amino acid sequence derived from any species (e.g., human, pig, cattle, dog, rat, mouse, guinea pigs, etc.). The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e. little, if any, of the antibodies generated by the IL-31 peptide immunogen constructs will be directed towards the Th epitope, thus allowing a very focused immune response directed to the targeted B cell epitope of the IL-31 fragment.

Th epitopes of the present disclosure include, but are not limited to, amino acid sequences derived from foreign pathogens, as exemplified in Table 2 (SEQ ID NOs: 14 to 42). Further, Th epitopes include idealized artificial Th epitopes and combinatorial idealized artificial Th epitopes (e.g., SEQ ID NOs: 15 and 22-28). The heterologous Th epitope peptides presented as a combinatorial sequence (e.g., SEQ ID NOs: 23-26), contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 23-26 which are shown in Table 2. Th epitope peptides of the present invention provide broad reactivity and immunogenicity to animals and patients from genetically diverse populations.

IL-31 peptide immunogen constructs comprising Th epitopes are produced simultaneously in a single solid-phase peptide synthesis in tandem with the IL-31 fragment. Th epitopes also include immunological analogues of Th epitopes. Immunological Th analogues include immune-enhancing analogs, cross-reactive analogues and segments of any of these Th epitopes that are sufficient to enhance or stimulate an immune response to the IL-31 fragments.

Functional immunologically analogues of the Th epitope peptides are also effective and included as part of the present invention. Functional immunological Th analogues can include conservative substitutions, additions, deletions and insertions of from one to about five amino acid residues in the Th epitope which do not essentially modify the Th-stimulating function of the Th epitope. The conservative substitutions, additions, and insertions can be accomplished with natural or non-natural amino acids, as described above for the IL-31 fragments. Table 2 identifies another variation of a functional analogue for Th epitope peptide. In particular. SEQ ID NOs: 15 and 22 of MvF1 and MvF2 Th are functional analogues of SEQ ID NOs: 25 and 27 of MvF4 and MvF5 in that they differ in the amino acid frame by the deletion (SEQ ID NOs: 15 and 22) or the inclusion (SEQ ID NOs: 25 and 27) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-4 Ths (SEQ ID NOs: 15, 22, 23, 25, and 27) and from Hepatitis Surface protein HBsAg 1-3 Ths (SEQ ID NOs: 24, 26, and 28).

The Th epitope in the IL-31 peptide immunogen construct can be covalently linked at either N- or C-terminal end of the IL-31 peptide. In some embodiments, the Th epitope is covalently linked to the N-terminal end of the IL-31 peptide. In other embodiments, the Th epitope is covalently linked to the C-terminal end of the IL-31 peptide. In certain embodiments, more than one Th epitope is covalently linked to the IL-31 fragment. When more than one Th epitope is linked to the IL-31 fragment, each Th epitope can have the same amino acid sequence or different amino acid sequences. In addition, when more than one Th epitope is linked to the IL-31 fragment, the Th epitopes can be arranged in any order. For example, the Th epitopes can be consecutively linked to the N-terminal end of the IL-31 fragment, or consecutively linked to the C-terminal end of the IL-31 fragment, or a Th epitope can be covalently linked to the N-terminal end of the IL-31 fragment while a separate Th epitope is covalently linked to the C-terminal end of the IL-31 fragment. There is no limitation in the arrangement of the Th epitopes in relation to the IL-31 fragment.

In some embodiments, the Th epitope is covalently linked to the IL-31 fragment directly. In other embodiments, the Th epitope is covalently linked to the IL-31 fragment through a heterologous spacer described in further detail below.

c. Heterologous Spacer

The disclosed IL-31 peptide immunogen constructs optionally contain a heterologous spacer that covalently links the B cell epitope from IL-31 to the heterologous T helper cell (Th) epitope.

As discussed above, the term “heterologous”, refers to an amino acid sequence that is derived from an amino acid sequence that is not part of, or homologous with, the wild-type sequence of IL-31. Thus, the natural amino acid sequence of IL-31 is not extended in either the N-terminal or C-terminal directions when the heterologous spacer is covalently linked to the B cell epitope from IL-31 because the spacer is heterologous to the IL-31 sequence.

The spacer is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer can provide structural features to the IL-31 peptide immunogen construct. Structurally, the spacer provides a physical separation of the Th epitope from the B cell epitope of the IL-31 fragment. The physical separation by the spacer can disrupt any artificial secondary structures created by joining the Th epitope to the B cell epitope. Additionally, the physical separation of the epitopes by the spacer can eliminate interference between the Th cell and/or B cell responses. Furthermore, the spacer can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences encoding flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 92), where Xaa is any amino acid, and preferably aspartic acid.

The spacer can also provide functional features to the IL-31 peptide immunogen construct. For example, the spacer can be designed to change the overall charge of the IL-31 peptide immunogen construct, which can affect the solubility of the peptide immunogen construct. Additionally, changing the overall charge of the IL-31 peptide immunogen construct can affect the ability of the peptide immunogen construct to associate with other compounds and reagents. As discussed in further detail below, the IL-31 peptide immunogen construct can be formed into a stable immunostimulatory complex with a highly charged oligonucleotide, such as CpG oligomers through electrostatic association. The overall charge of the IL-31 peptide immunogen construct is important for the formation of these stable immunostimulatory complexes.

Chemical compounds that can be used as a spacer include, but are not limited to, (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. Non-naturally occurring amino acids include, but are not limited to, ε-N Lysine, β-alanine, omithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

The spacer in the IL-31 peptide immunogen construct can be covalently linked at either N- or C-terminal end of the Th epitope and the IL-31 peptide. In some embodiments, the spacer is covalently linked to the C-terminal end of the Th epitope and to the N-terminal end of the IL-31 peptide. In other embodiments, the spacer is covalently linked to the C-terminal end of the IL-31 peptide and to the N-terminal end of the Th epitope. In certain embodiments, more than one spacer can be used, for example, when more than one Th epitope is present in the peptide immunogen construct. When more than one spacer is used, each spacer can be the same as each other or different. Additionally, when more than one Th epitope is present in the peptide immunogen construct, the Th epitopes can be separated with a spacer, which can be the same as, or different from, the spacer used to separate the Th epitope from the B cell epitope. There is no limitation in the arrangement of the spacer in relation to the Th epitope or the IL-31 fragment.

In certain embodiments, the heterologous spacer is a naturally occurring amino acid or a non-naturally occurring amino acid. In other embodiments, the spacer contains more than one naturally occurring or non-naturally occurring amino acid. In specific embodiments, the spacer is Lys-, Gly-, Lys-Lys-Lys-, (α, ε-N)Lys, or ε-N-Lys-Lys-Lys-Lys (SEQ ID NO: 91).

d. Specific Embodiments of the IL-31 Peptide Immunogen Construct

In certain embodiments, the IL-31 peptide immunogen construct can be represented by the formulae:

(Th)ₘ-(A)ₙ-(IL-31 fragment)-X

or

(IL-31 fragment)-(A)ₙ-(Th)ₘ-X

wherein

Th is a heterologous T helper epitope:

A is a heterologous spacer;

(IL-31 fragment) is a B cell epitope having about 15 to about 75 amino acid residues from SEQ ID NO: 1 or SEQ ID NO: 2:

X is an α-COOH or α-CONH₂ of an amino acid;

m is from 1 to about 4; and

n is from 0 to about 10.

In certain embodiments, the heterologous Th epitope in the IL-31 peptide immunogen construct has an amino acid sequence selected from any of SEQ ID NOs: 14-42 and combinations thereof, shown in Table 2. In specific embodiments, the Th epitope has an amino acid sequence selected from any of SEQ ID NOs: 22-28. In certain embodiments, the IL-31 peptide immunogen construct contains more than one Th epitope.

In certain embodiments, the optional heterologous spacer is selected from any of Lys-, Gly-, Lys-Lys-Lys-, (α, ε-N)Lys, ε-N-Lys-Lys-Lys-Lys (SEQ ID NO: 91), and combinations thereof. In specific embodiments, the heterologous spacer is ε-N-Lys-Lys-Lys-Lys (SEQ ID NO: 91).

In certain embodiments, the IL-31 fragment has about 15 to about 65 amino acid residues from SEQ ID NO: 1 or SEQ ID NO: 2. In specific embodiments, the IL-31 fragment has an amino acid sequence represented by SEQ ID NOs: 1-13 and 93 to 98, as shown in Table 1.

In certain embodiments, the IL-31 peptide immunogen construct has an amino acid sequence selected from any of SEQ ID NOs: 43-90 and 99-105, as shown in Table 3.

e. Variants, Homologues, and Functional Analogues

Variants and analogs of the above immunogenic peptides that induce and/or cross-react with antibodies to the preferred epitopes of IL-31 protein can also be used. Analogs, including allelic, species, and induced variants, typically differ from naturally occurring peptides at one, two, or a few positions, often by virtue of conservative substitutions. Analogs typically exhibit at least 80 or 90% sequence identity with natural peptides. Some analogs also include unnatural amino acids or modifications of N- or C-terminal amino acids at one, two, or a few positions.

Variants that are functional analogues can have a conservative substitution in an amino acid position; a change in overall charge: a covalent attachment to another moiety; or amino acid additions, insertions, or deletions; and/or any combination thereof.

Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

In a particular embodiment, the functional analogue has at least 50% identity to the original amino acid sequence. In another embodiment, the functional analogue has at least 80% identity to the original amino acid sequence. In yet another embodiment, the functional analogue has at least 85% identity to the original amino acid sequence. In still another embodiment, the functional analogue has at least 90% identity to the original amino acid sequence.

Variants also include variations to the phosphorylated residues. For example, variants can include different residues within the peptides that are phosphorylated. Variant immunogenic IL-31 peptides can also include pseudo-phosphorylated peptides. The pseudo-phosphorylated peptides are generated by substituting one or more of the phosphorylated serine, threonine, and tyrosine residues of the IL-31 peptides with acidic amino acid residues such as glutamic acid and aspartic acid.

### Compositions

The present disclosure also provides compositions comprising the disclosed IL-31 immunogen construct.

a. Peptide Compositions

Compositions containing a disclosed IL-31 peptide immunogen construct can be in liquid or solid form. Liquid compositions can include water, buffers, solvents, salts, and/or any other acceptable reagent that does not alter the structural or functional properties of the IL-31 peptide immunogen construct. Peptide compositions can contain one or more of the disclosed IL-31 peptide immunogen constructs.

b. Pharmaceutical Compositions

The present disclosure is also directed to pharmaceutical compositions containing the disclosed IL-31 peptide immunogen construct.

Pharmaceutical compositions can contain carriers and/or other additives in a pharmaceutically acceptable delivery system. Accordingly, pharmaceutical compositions can contain a pharmaceutically effective amount of an IL-31 peptide immunogen construct together with pharmaceutically-acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the IL-31 peptide immunogen construct without having any specific antigenic effect itself. Adjuvants used in the pharmaceutical composition can include oils, oil emulsions, aluminum salts, calcium salts, immune stimulating complexes, bacterial and viral derivatives, virosomes, carbohydrates, cytokines, polymeric microparticles. In +certain embodiments, the adjuvant can be selected from alum (potassium aluminum phosphate), aluminum phosphate (e.g. ADJU-PHOS®), aluminum hydroxide (e.g. ALHYDROGEL®), calcium phosphate, incomplete Freund's adjuvant (IFA), Freund's complete adjuvant, MF59, adjuvant 65, Lipovant, ISCOM, liposyn, saponin, squalene, L121, EMULSIGEN®, monophosphoryl lipid A (MPL), Quil A, QS21, MONTANIDE® ISA 35, ISA 50V, ISA 50V2, ISA 51, ISA 206, ISA 720, liposomes, phospholipids, peptidoglycan, lipopolysaccahrides (LPS), ASO1, ASO2, ASO3, ASO4, AF03, lipophilic phospholipid (lipid A), gamma inulin, algammulin, glucans, dextrans, glucomannons, galactomannans, levans, xylans, dimethyldioctadecylammonium bromide (DDA), as well as the other adjuvants and emulsifiers.

In some embodiments, the pharmaceutical composition contains MONTANIDET™ ISA 51 (an oil adjuvant composition comprised of vegetable oil and mannide oleate for production of water-in-oil emulsions), TWEEN® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e. w/o/w) emulsion with EMULSIGEN or EMULSIGEN D as the adjuvant.

Pharmaceutical compositions can also include pharmaceutically acceptable additives or excipients. For example, pharmaceutical compositions can contain antioxidants, binders, buffers, bulking agents, carriers, chelating agents, coloring agents, diluents, disintegrants, emulsifying agents, fillers, gelling agents, pH buffering agents, preservatives, solubilizing agents, stabilizers, and the like.

Pharmaceutical compositions can be formulated as immediate release or for sustained release formulations. Additionally the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the IL-31 peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for intravenous, subcutaneous, intradermal, or intramuscular administration. Pharmaceutical compositions suitable for other modes of administration can also be prepared, including oral and intranasal applications.

Pharmaceutical compositions can also formulated in a suitable dosage unit form. In some embodiments, the pharmaceutical composition contains from about 0.5 μg to about 1 mg of the IL-31 peptide immunogen construct per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight and general health of the subject as is well known in the therapeutic arts.

In some embodiments, the pharmaceutical composition contains more than one IL-31 peptide immunogen construct. A pharmaceutical composition containing a mixture of more than one IL-31 peptide immunogen construct to allow for synergistic enhancement of the immunoefficacy of the constructs. Pharmaceutical compositions containing more than one IL-31 peptide immunogen construct can be more effective in a larger genetic population due to a broad MHC class II coverage thus provide an improved immune response to the IL-31 peptide immunogen constructs.

In some embodiments, the pharmaceutical composition contains an IL-31 peptide immunogen construct selected from SEQ ID NOs: 43-90 and 99-105 (Table 3), as well as homologues, analogues and/or combinations thereof. In specific embodiments, pharmaceutical compositions contain an IL-31 peptide immunogen construct selected from SEQ ID NOs: 43-90 and 99-105 (Table 3), and any combination thereof.

Pharmaceutical compositions containing an IL-31 peptide immunogen construct can be used to elicit an immune response and produce antibodies in a host upon administration.

c. Immunostimulatory Complexes

The present disclosure is also directed to pharmaceutical compositions containing an IL-31 peptide immunogen construct in the form of an immunostimulatory complex with a CpG oligonucleotide, such as CpG1 (SEQ ID NO: 108), CpG2 (SEQ ID NO: 109), or CpG3 (SEQ ID NO: 110). Such immunostimulatory complexes are specifically adapted to act as an adjuvant and as a peptide immunogen stabilizer. The immunostimulatory complexes are in the form of a particulate, which can efficiFINently present the IL-31 peptide immunogen to the cells of the immune system to produce an immune response. The immunostimulatory complexes may be formulated as a suspension for parenteral administration. The immunostimulatory complexes may also be formulated in the form of w/o emulsions, as a suspension in combination with a mineral salt or with an in-situ gelling polymer for the efficient delivery of the IL-31 peptide immunogen to the cells of the immune system of a host following parenteral administration.

The stabilized immunostimulatory complex can be formed by complexing an IL-31 peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the IL-31 peptide immunogen construct is designed to contain a cationic portion that is positively charged at a pH in the range of 5.0 to 8.0. The net charge on the cationic portion of the IL-31 peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the IL-31 peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the IL-31 peptide immunogen construct is the heterologous spacer. In certain embodiments, the cationic portion of the IL-31 peptide immunogen construct has a charge of +4 when the spacer sequence is (α, ε-N)Lys, ε-N-Lys-Lys-Lys-Lys (SEQ ID NO: 91).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. Preferably, the CpG immunostimulatory single-stranded DNA molecules contain 18-48 nucleotide bases, with the number of repeats of CpG motif in the range of 3 to 8.

More preferably the anionic oligonucleotide is represented by the formula: 5′ X¹CGX² 3′ wherein C and G are unmethylated; and X¹ is selected from the group consisting of A (adenine), G (guanine) and T (thymine); and X² is C (cytosine) or T (thymine). Or, the anionic oligonucleotide is represented by the formula: 5′ (X³)₂CG(X⁴)₂ 3′ wherein C and G are unmethylated; and X³ is selected from the group consisting of A, T or G; and X⁴ is C or T.

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

### Antibodies

The present disclosure also provides antibodies elicited by the IL-31 peptide immunogen construct.

The disclosed IL-31 peptide immunogen constructs, comprising an IL-31 fragment, heterologous Th epitope, and optional heterologous spacer, are capable of eliciting an immune response and the production of antibodies when administered to a host. The design of the IL-31 peptide immunogen constructs can break tolerance to self-IL-31 and elicit the production of site-specific antibodies that recognize conformational, not linear, epitopes.

The antibodies produced by the IL-31 peptide immunogen constructs recognize and bind to IL-31 in the forms of monomers, dimers, trimers, and oligomers.

The resulting immune responses from animals immunized with IL-31 peptide immunogen constructs of the present invention demonstrated the ability of the constructs to produce potent site-directed antibodies that are reactive with IL-31.

### Methods

The present disclosure is also directed to methods for making and using the IL-31 peptide immunogen constructs, compositions, and pharmaceutical compositions.

a. Methods for Manufacturing the IL-31 Peptide Immunogen Construct

The IL-31 peptide immunogen constructs of this disclosure can be made by chemical synthesis methods well known to the ordinarily skilled artisan (see, e.g., Fields et al., Chapter 3 in Synthetic Peptides: A User's Guide, ed. Grant, W H. Freeman & Co., New York, N.Y., 1992, p. 77). The IL-31 peptide immunogen constructs can be synthesized using the automated Merrifield techniques of solid phase synthesis with the α-NH₂ protected by either t-Boc or F-moc chemistry using side chain protected amino acids on, for example, an Applied Biosystems Peptide Synthesizer Model 430A or 431. Preparation of IL-31 peptide immunogen constructs comprising combinatorial library peptides for T epitopes can be accomplished by providing a mixture of alternative amino acids for coupling at a given variable position.

After complete assembly of the desired IL-31 peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to one of ordinary skill in the art.

The quality of peptides produced by this chemical process can be controlled and defined and, as a result, reproducibility of IL-31 peptide immunogen constructs, immunogenicity, and yield can be assured. Detailed description of the manufacturing of the IL-31 peptide immunogen construct through solid phase peptide synthesis is shown in Example 1.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically-derived drugs. Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The IL-31 peptide immunogen constructs can also be made using recombinant DNA technology including nucleic acid molecules, vectors, and/or host cells. As such, nucleic acid molecules encoding the IL-31 peptide immunogen construct and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the IL-31 peptide immunogen construct and immunologically functional analogues thereof. For example, methods can include a step of incubating a host cell containing an expression vector containing a nucleic acid molecule encoding an IL-31 peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

In some embodiments, the IL-31 peptide immunogen construct can be expressed in specific E. coli strains that allow for lipidation to make an IL-31 lipoprotein, as described in U.S. Pat. No. 8,426,163, which is incorporated herein by reference in its entirety. In certain embodiments, the E. coli strains used are resistant to the toxic effects induced by over-expression of exogenous proteins, in particular, membrane proteins. Such E. coli strains can be identified/generated by the methods described in U.S. Pat. No. 6,361,966, which is incorporated herein by reference in its entirety. Examples of E. coli strains that are capable of producing a lipoprotein in lipidated form include, but are not limited to. C43(DE3) (ECCC B96070445). C41(DE3) (ECCC B96070444), CO₂₁₄(DE3), DK8(DE3)S (NCIMB 40885), and C2014(DE3) (NCIMB 40884). A lipidated form of IL-31 can be expressed in one of the E. coli strains noted above via conventional recombinant technology. Briefly, a DNA fragment encoding IL-31 is obtained from its native source via, e.g., PCR amplification, and optionally modified to optimize codon usage in E. coli. The DNA fragment is then inserted into an E. coli expression vector to produce an expression plasmid. Preferably, expression of the IL-31 lipoprotein is driven by a strong promoter, e.g., T7, T5, T3, or SP6, which can be inducible, e.g., by IPTG. The expression plasmid is then introduced into a selected E. coli strain and positive transformants are cultured under suitable conditions for protein expression. The lipoprotein thus expressed can be isolated from the E. coli cells and its lipidation status can be confirmed via methods known in the art, e.g., immunoblotting with an anti-lipoprotein antibody or mass spectrometry.

b. Methods for the Manufacturing of Immunostimulatory Complexes

Various exemplary embodiments also encompass methods of producing the Immunostimulatory complexes comprising IL-31 peptide immunogen constructs and CpG oligodeoxynucleotide (ODN) molecule. Stabilized immunostimulatory complexes (ISC) are derived from a cationic portion of the IL-31 peptide immunogen construct and a polyanionic CpG ODN molecule. The self-assembling system is driven by electrostatic neutralization of charge. Stoichiometry of the molar charge ratio of cationic portion of the IL-31 peptide immunogen construct to anionic oligomer determines extent of association. The non-covalent electrostatic association of IL-31 peptide immunogen construct and CpG ODN is a completely reproducible process. The peptide/CpG ODN immunostimulatory complex aggregates, which facilitate presentation to the “professional” antigen-presenting cells (APC) of the immune system thus further enhancing of the immunogenicity of the complexes. These complexes are easily characterized for quality control during manufacturing. The peptide/CpG ISC are well tolerated in vivo. This novel particulate system comprising CpG ODN and IL-31 fragment derived peptide immunogen constructs was designed to take advantage of the generalized B cell mitogenicity associated with CpG ODN use, yet promote balanced Th-1/Th-2 type responses.

The CpG ODN in the disclosed pharmaceutical compositions is 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen-presenting cells (APC). Consequently, such formulations are novel conceptually and offer potential advantages by promoting the stimulation of immune responses by alternative mechanisms.

c. Methods for the Manufacturing Pharmaceutical Compositions

Various exemplary embodiments also encompass pharmaceutical compositions containing IL-31 peptide immunogen constructs. In certain embodiments, the pharmaceutical compositions employ water in oil emulsions and in suspension with mineral salts.

In order for a pharmaceutical composition to be used by a large population and with prevention of IL-31 aggregation also being part of the goal for administration, safety becomes another important factor for consideration. Despite the use of water-in-oil emulsions in humans for many formulations in clinical trials, Alum remains the major adjuvant for use in formulations due to its safety. Alum or its mineral salts Aluminum phosphate (ADJUPHOS) are, therefore, frequently used as adjuvants in preparation for clinical applications.

Other adjuvants and immunostimulating agents include 3 De-O-acylated monophosphoryl lipid A (MPL) or 3-DMP, polymeric or monomeric amino acids, such as polyglutamic acid or polylysine. Such adjuvants can be used with or without other specific immunostimulating agents, such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′ dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include MF59 (see WO 90/14837 to Van Nest et al., which is hereby incorporated by reference in its entirety), containing 5% Squalene, 0.5% TWEEN 80, and 0.5% Span 85 (optionally containing various amounts of MTP-PE) formulated into submicron particles using a microfluidizer; SAF, containing 10% Squalene, 0.4% TWEEN 80, 5% pluronic-blocked polymer L121, and thr-MDP, either microfluidized into a submicron emulsion or vortexed to generate a larger particle size emulsion; and the Ribi™ adjuvant system (RAS) (Ribi ImmunoChem, Hamilton, Mont.) containing 2% squalene, 0.2% TWEEN 80, and one or more bacterial cell wall components selected from the group consisting of monophosphoryllipid A (MPL), trehalose dimycolate (TDM), and cell wall skeleton (CWS), preferably MPL+CWS (Detox™). Other adjuvants include Complete Freund's Adjuvant (CFA), Incomplete Freund's Adjuvant (IFA), and cytokines, such as interleukins (IL-1, IL-2, and IL-12), macrophage colony stimulating factor (M-CSF), and tumor necrosis factor (TNF).

The choice of an adjuvant depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, alum, MPL or Incomplete Freund's adjuvant (Chang et al., Advanced Drug Delivery Reviews 32:173-186 (1998), which is hereby incorporated by reference in its entirety) alone or optionally all combinations thereof are suitable for human administration.

The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, non-immunogenic stabilizers, and the like.

Pharmaceutical compositions can also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Additionally, these carriers can function as immunostimulating agents (i.e., adjuvants).

The pharmaceutical compositions of the present invention can further include a suitable delivery vehicle. Suitable delivery vehicles include, but are not limited to viruses, bacteria, biodegradable microspheres, microparticles, nanoparticles, liposomes, collagen minipellets, and cochleates.

d. Methods Using Pharmaceutical Compositions

The present disclosure also includes methods of using pharmaceutical compositions containing IL-31 peptide immunogen constructs.

In certain embodiments, the pharmaceutical compositions containing IL-31 peptide immunogen constructs can be used for the treatment and/or prevention of a pruritic condition and/or an allergic condition, such as atopic dermatitis.

### SPECIFIC EMBODIMENTS

(1) An IL-31 peptide immunogen construct can be represented by the formulae:

(Th)ₘ-(A)ₙ-(IL-31 fragment)-X

or

(IL-31 fragment)-(A)ₙ-(Th)ₘ-X

wherein

Th is a heterologous T helper epitope;

A is a heterologous spacer;

(IL-31 fragment) is a B cell epitope having about 15 to about 75 amino acid residues from SEQ ID NO: 1 or SEQ ID NO: 2:

X is an α-COOH or α-CONH₂ of an amino acid;

m is from 1 to about 4; and

n is from 0 to about 10.

(2) The IL-31 peptide immunogen construct according to (1), wherein the IL-31 fragment is selected from the group consisting of SEQ ID NOs: 1-13 and 93-98.
(3) The IL-31 peptide immunogen construct according to any of (1) or (2), wherein the Th epitope is selected from the group consisting of SEQ ID NOs: 14-42.
(4) The IL-31 peptide immunogen construct according to (1), wherein the peptide immunogen construct is selected from the group consisting of SEQ ID NOs: 43-90 and 99 to 105.
(5) An IL-31 peptide immunogen construct comprising:

a B cell epitope comprising about 15 to about 75 amino acid residues from the full-length IL-31 protein sequence of SEQ ID NO: 1 or SEQ ID NO: 2;

a T helper epitope comprising an amino acid sequence selected from the group consisting of SEQ ID NOs: 14-42; and

an optional heterologous spacer selected from the group consisting of an amino acid, Lys-, Gly-, Lys-Lys-Lys-, (α, ε-N)Lys, and ε-N-Lys-Lys-Lys-Lys (SEQ ID NO: 91),

wherein the B cell epitope is covalently linked to the T helper epitope directly or through the optional heterologous spacer.

(6) The IL-31 peptide immunogen construct of (5), wherein the B cell epitope is selected from the group consisting of SEQ ID NOs: 1-13 and 93-98.
(7) The IL-31 peptide immunogen construct of (5), wherein the T helper epitope is selected from the group consisting of SEQ ID NOs: 14-42.
(8) The IL-31 peptide immunogen construct of (5), wherein the optional heterologous spacer is (α, ε-N)Lys or ε-N-Lys-Lys-Lys-Lys (SEQ ID NO: 91).
(9) The IL-31 peptide immunogen construct of (5), wherein the T helper epitope is covalently linked to the amino terminus of the B cell epitope.
(10) The IL-31 peptide immunogen construct of (5), wherein the T helper epitope is covalently linked to the amino terminus of the B cell epitope through the optional heterologous spacer.
(11) A composition comprising a peptide immunogen construction according to any of (1) to (4).
(12) A pharmaceutical composition comprising:

a, a peptide immunogen construct according to any of (1) to (4); and

b, and a pharmaceutically acceptable delivery vehicle and/or adjuvant.

(13) The pharmaceutical composition of (12), wherein

a, the IL-31 peptide immunogen construct is selected from the group consisting of SEQ ID NOs: 43-90 and 99-105; and

b, the IL-31 peptide immunogen construct is mixed with an CpG oligodeoxynucleotide (ODN) to form a stabilized immunostimulatory complex.

(14) An isolated antibody or epitope-binding fragment thereof that specifically binds to the B cell epitope of the IL-31 peptide immunogen construct according to any of (1) to (10).
(15) The isolated antibody or epitope-binding fragment thereof according to (14) bound to the IL-31 peptide immunogen construct.
(16) An isolated antibody or epitope-biding fragment thereof that specifically binds to the B cell epitope of the IL-31 peptide immunogen construct according to any of (1) to (10).
(17) A composition comprising the isolated antibody or epitope-binding fragment thereof according to any of claims (14) to (16).

### Example 1

### Synthesis of IL-31 Related Peptides and Preparation of Formulations Thereof

a. IL-31 Fragments

Methods for synthesizing designer IL-31 fragments that were included in the development effort of IL-31 peptide immunogen constructs are described. The peptides were synthesized in small-scale amounts that are useful for serological assays and laboratory pilot and field studies, which are useful for analyzing pharmaceutical compositions. A large repertoire of IL-31 related antigenic peptides having sequences with lengths from approximately 15 to approximately 75 amino acids (Table 1) were designed for the screening and selection of the most optimal peptide constructs for use in an efficacious IL-31 peptide immunogen construct.

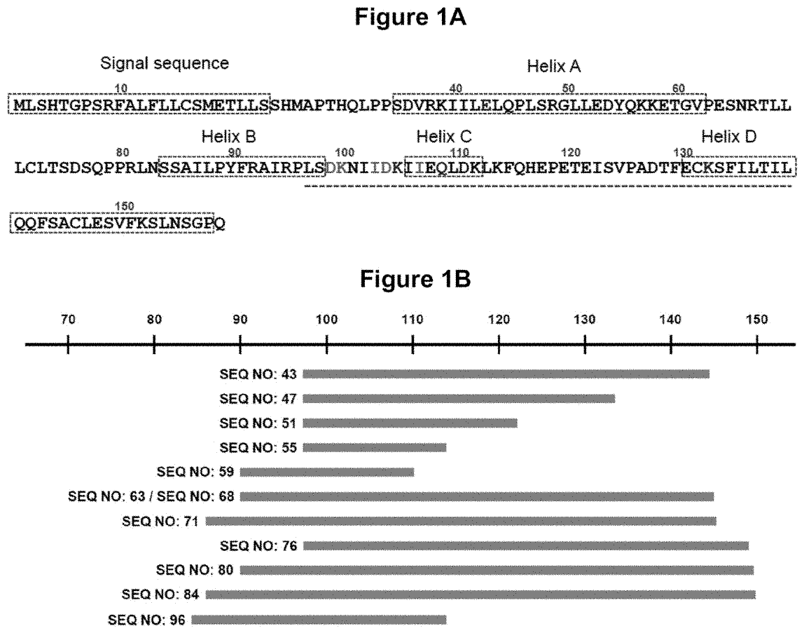

Amino acid sequences from the full length canine IL-31 (SEQ ID NO: 1) (Figure A) and human IL-31 (SEQ ID NO: 2) proteins were used. The IL-31 fragments employed for epitope mapping in various serological assays are identified in Table 1 (SEQ ID NOs: 3-13 and 93-98) and the relative sequence alignment of the IL-31 fragments to the full-length sequence is shown in FIG. 1B. Selected IL-31 fragments (SEQ ID NOs: 3-13 and 93-98) were made into IL-31 peptide immunogen constructs by synthetically linking to a carefully designed helper T cell (Th) epitope derived from pathogen proteins including UBITh®1 and UBITh®2 identified in Table 2 (SEQ ID NOs: 26 and 25, respectively). The Th epitopes were used either in a single sequence (UBITh®1) or a combinatorial library (UBITh®3) to enhance the immunogenicity of their respective IL-31 peptide immunogen constructs.

Representative IL-31 peptide immunogen constructs are identified in Table 3 (SEQ ID NOs: 43-90 and 99-105). The IL-31 peptide immunogen constructs that were synthesized and evaluated are shown in Table 3 with the peptide code identified.

b. Typical Synthesis of Peptides

Peptides used for immunogenicity studies or related serological tests for detection and/or measurement of anti-L-31 antibodies are typically synthesized on a small scale using F-moc chemistry by peptide synthesizers of Applied BioSystems Models 430A, 431 and/or 433. Each peptide is produced by an independent synthesis on a solid-phase support, with F-moc protection at the N-terminus and side chain protecting groups of trifunctional amino acids. Completed peptides are cleaved from the solid support and side chain protecting groups are removed by 90% Trifluoroacetic acid (TFA). Synthetic peptide preparations are evaluated by Matrix-Assisted Laser Desorption/Ionization-Time-Of-Flight (MALDI-TOF) Mass Spectrometry to ensure correct amino acid content. Each synthetic peptide is also evaluated by Reverse Phase HPLC (RP-HPLC) to confirm the synthesis profile and concentration of the preparation. Despite rigorous control of the synthesis process (including stepwise monitoring of the coupling efficiency), peptide analogues are also produced due to unintended events during elongation cycles, including amino acid insertion, deletion, substitution, and premature termination. Thus, synthesized preparations typically include multiple peptide analogues along with the targeted peptide. Despite the inclusion of such unintended peptide analogues, the resulting synthesized peptide preparations are nevertheless suitable for use in immunological applications including immunodiagnosis (as antibody capture antigens) and pharmaceutical compositions (as peptide immunogens). Typically, such peptide analogues, either intentionally designed or generated through synthetic process as a mixture of byproducts, are frequently as effective as a purified preparation of the desired peptide, as long as a discerning QC procedure is developed to monitor both the manufacturing process and the product evaluation process to guarantee the reproducibility and efficacy of the final product employing these peptides. Large scale peptide syntheses in the multi-hundred to kilo-gram quantities are conducted on a customized automated peptide synthesizer UBI2003 or the like at 15 mmole to 150 mmole scale. For active ingredients to be used in the final pharmaceutical composition for clinical trials, IL-31 peptide constructs are purified by preparative RP-HPLC under a shallow elution gradient and characterized by MALDI-TOF mass spectrometry, amino acid analysis and RP-HPLC for purity and identity.

### Example 2

### Immunization of Guinea Pigs with IL-31 Peptide Immunogen Constructs for Immunogenicity Assessment

a. Animals

Guinea pigs (300-350 g in weight) were used for the immunization to assess the immunogenicity of representative designer canine IL-31 peptide immunogen constructs (SEQ ID NOs: 43, 47, 51, 55, and 59). Fifteen (15) guinea pigs were divided into groups with 3 animals per group and an initial immunization with 400 μg of peptide followed by 4 boosters of 100 μg on 3, 6, 9 and 12 weeks post initial immunization (wpi), as summarized in Table 4. Blood samples were collected at 0, 3, 6, 9, 12 and 15 wpi for titer of anti-IL-31 antibodies measurement and for assessment of other functional properties of the antibodies present in the corresponding immune sera.

b. Formulation

In general, the formulations used for the immunization contained the following:

1. 400 μg of L-31 peptide immunogen construct

2. CpG at a peptide:CpG ratio of 0.7:1

3. 0.2% TWEEN 80

c. Titration of Serum Anti-IL-31 Antibodies by ELISA

The titration of serum anti-IL-31 antibodies elicited by the IL-31 peptide immunogen constructs were evaluated according to the following protocol:

Wells of 96 well plates were coated with 100 μl of canine IL-31 recombinant protein (rcIL-31)(50 ng/well) or IL-31 B epitope peptide (SEQ ID NOs: 3-13 and 93-98) at 0.2 μg/0.1 mL/well for 1 hour at 37° C. at 1 μg/ml in 50 mM carbonated-bicarbonate buffer, pH9.6.

The rcIL-31 coated wells were incubated with 200 μl of 1% BSA in PBS at 37° C. for 1 hour to block non-specific protein binding, followed by three washes with PBS containing 0.05% TWEEN 20 and then dried. One hundred microliters (100 μl) of the serial diluted sera samples from immunized guinea pigs were added to each well and to react for 1 hour at 37° C. The wells were then washed with PBS containing 0.05% TWEEN 20 five times and dried. 100 μl of peroxidase-labeled goat anti-guinea pig IgG with optimal dilution prepared in PBS containing 1% BSA and 0.05% TWEEN 20 was added to each well and incubated at 37° C. for 1 hour. After the incubation, wells were washed with PBS containing 0.05% TWEEN 20 six times and reacted with 100 μl of TMB substrate for another 15 minutes. Reactions were stopped by 2N H₂SO₄ and determined the absorbance at 450 nm.

For the determination of antibodies titer in guinea pigs that received IL-31 peptide-based vaccine formulation, a 10-fold serial dilution of sera from 1:100 or more, was performed for initial screening on the representative target “IL-31” B epitope peptides with titer expressed as Log₁₀. For further analysis of sera cross-reactive with recombinant canine IL-31, a 2-fold serial dilution of sera from 1:100 to 1:54,248,800 was performed on the ELISA and the titer expressed as Log EC₅₀.

d. Results

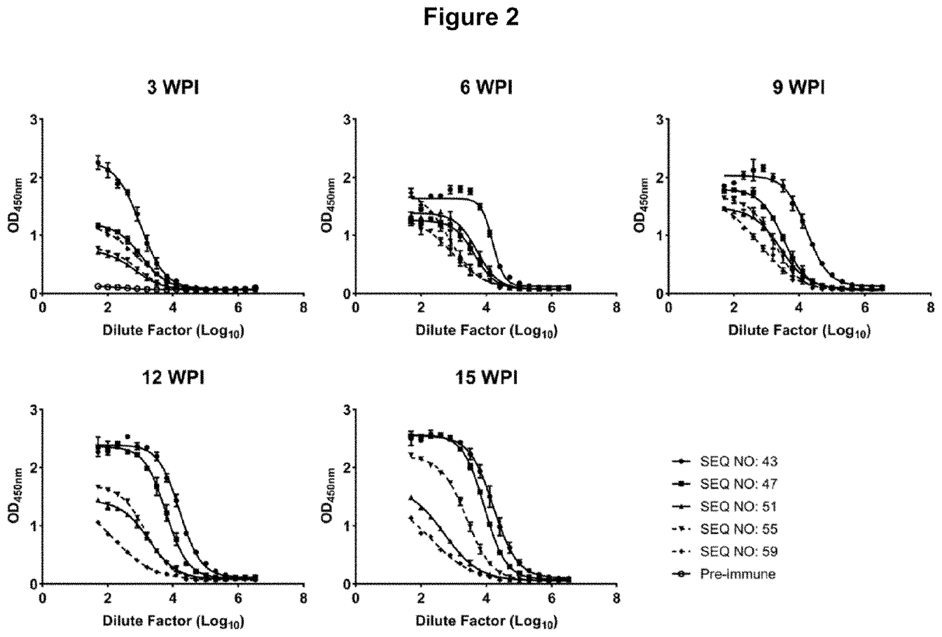

The results from the immunogenicity assessment with respective titers against the target IL-31 B epitope peptide (SEQ ID NOs: 43, 47, 51, 55, and 59) are shown in Table 5. The antibody titers (Log₁₀ EC₅₀) to recombinant canine IL-31 for each of the IL-31 peptide immunogen constructs are shown in Table 6 and FIG. 2.

High immunogenicity was found with these carefully designed canine IL-31 peptide immunogen constructs where high titer antibodies were elicited upon one single administration at 3 wpi towards the IL-31 region of AA90-AA144 (SEQ ID NO: 8) covered by peptides of SEQ ID NOs: 3 to 7. High cross-reactivity towards canine rIL-31 was found with antibodies elicited by carefully designed IL-31 peptide immunogen constructs (SEQ ID NOs: 43, 47, 51, 55, and 59).

### Example 3

### Production, Purification and Characterization of Canine IL-31 for Use in Biological Assays

a. Expression Construct

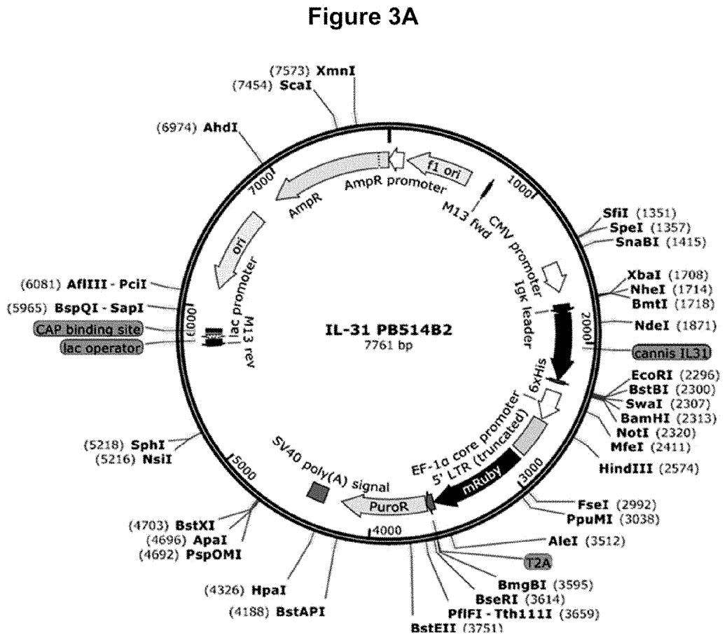

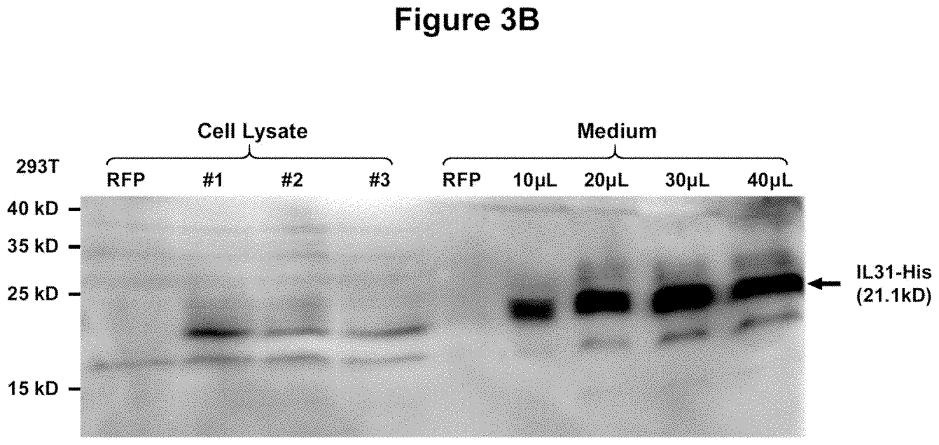

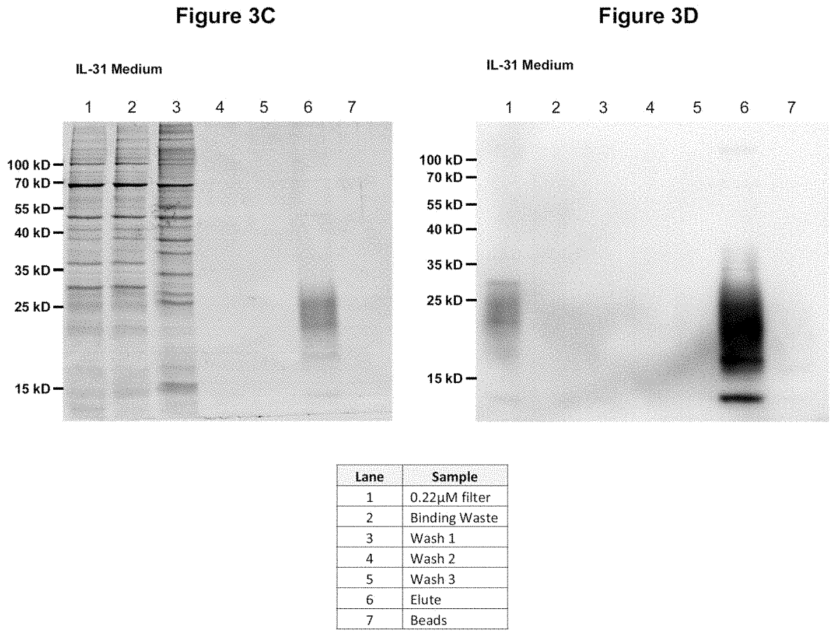

The sequence of canine 11-31 was identified by using the NCBIs genome resources (website: ncbi.nlm.nih.gov). An expression construct was created with the full-length canine IL-31 gene containing an C-terminal 6-His tag for detection and purification (see FIGS. 3A and 3B). The sequence confirmed plasmid was used to transfect in Expi293 cells and the expressed IL-31 would be secreted into culture medium. The medium was collected following 72 hours transfection and purification of the recombinant proteins was carried out using a nickel cobalt resin. After medium contained with secreted form IL-31 binding to the resin, the column was washed by wash buffer contained with 50 mM Tris buffer, pH8.0, 500 mM NaCl and 20 mM Imidazole three times within 10 fold of column volume. Then recombinant IL-31 was eluted by elution buffer contained with 50 mM Tris buffer, pH8.0, 500 mM NaCl and 250 mM Imidazole in 5 fold of column volume. Eluted fraction was analyzed by SDS-PAGE and Westem blots probed with an anti-His antibody.

b. Results

Coomassie blue staining of His-tagged IL-31 protein expression and purification run on a 12% Bis Tris SDS PAGE (FIG. 3C). Western blot of the SDS-PAGE gel is shown in FIG. 3C, probed with an anti-His-tag antibody (FIG. 3D). This 21.1 KD protein secreted in the medium was identified and purified for use in the following assays and as a reference protein for immunogenicity and cross-reactivity assessment.

### Example 4

### IL-31-Mediated pSTAT3 Signaling in a Cell-Based Assay

a. Assay

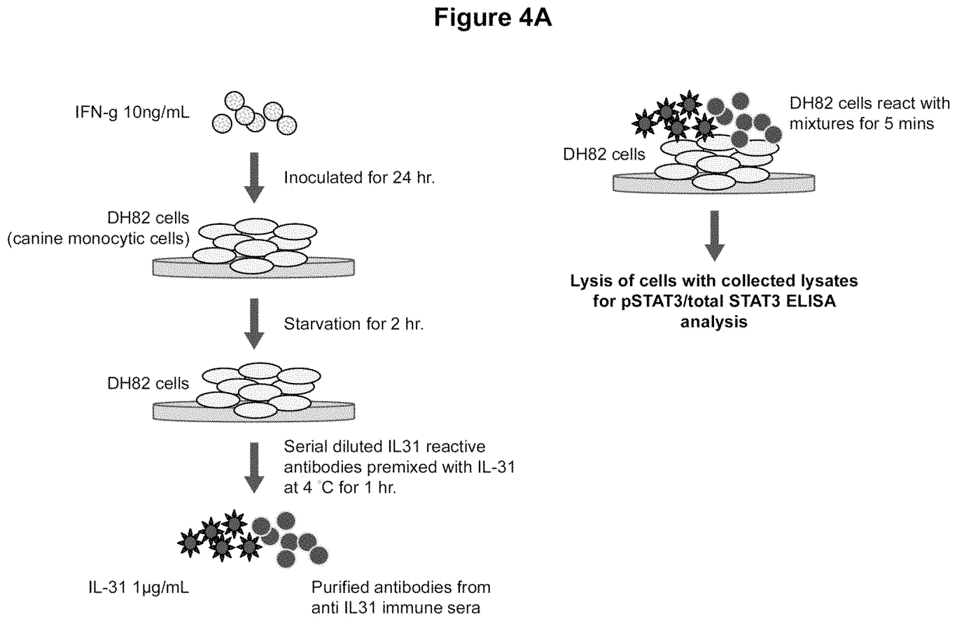

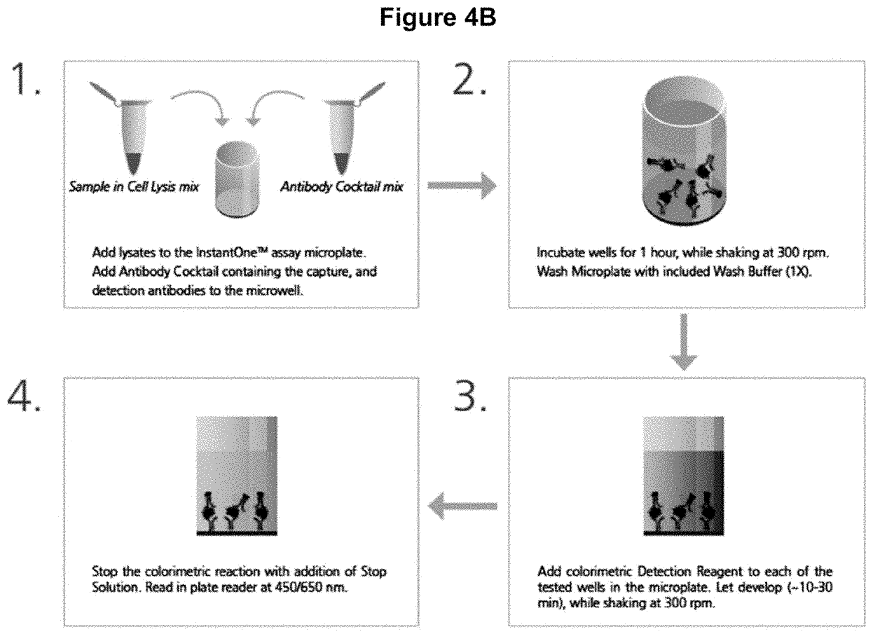

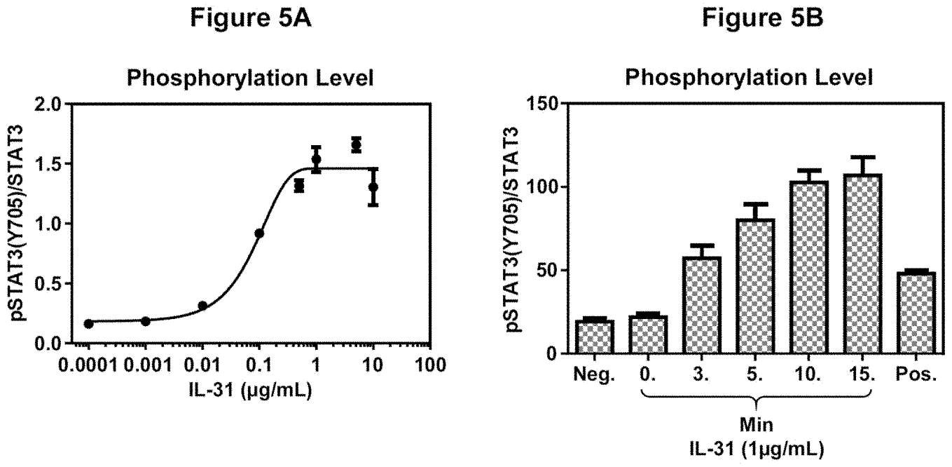

The in vitro IL-31 induced signal transduction functional inhibition assay using IL-31 antibodies elicited by the IL-31 peptide immunogen constructs is shown in FIGS. 4A and 4B and FIGS. 5A and 5B.

b. IL-31 Mediated nSTAT3 Signaling

DH-82 cells were plated into 96-well flat-bottomed cell culture plates at a density of 1×10⁵ cells per well in MEM growth media containing 15% heated-inactivated fetal bovine serum, 2 mmol/L GlutaMax, 1 mmol/L sodium pyruvate, 50 mg/L gentamicin and canine interferon-γ at 20 ng/mL for 16 hours at 37° C. in humidified air supplemented with 5% CO₂. Cells were serum starved for two hours prior to IL-31 treatment to increase IL-31 receptor expression. Following the pre-treatment, recombinant canine IL-31 was added at different doses (from 0.0001 to 10 ng/mL) for 5 minutes (FIG. 5A) or at 1 μg/mL for 0, 3, 5, 10 and 15 minutes (FIG. 5B). After the inoculation, cells were lysed with 20% final volume of Cell Lysis Mix (5×), and shaken at 300 rpm at 37° C. for 10 min. 50 μl of cell lysates were used to evaluate pSTAT3. 50 μl of peroxidase-labeled anti-pSTAT3(y705) or peroxidase-labeled anti-STAT3, antibodies were added into testing wells and to react for 1 hour at 37° C. Then wells were washed with PBS containing 0.05% TWEEN 20 five times and reacted with 100 μl of TMB substrate for another 15 minutes. Reactions were stopped by 2N H₂SO₄ and determined the absorbance at 450 nm.

c. Results

FIGS. 5A and 5B illustrate graphs of pSTAT3 signaling in canine DH-82 monocytes induced by canine IL-31 produced from expi293 cells. FIG. 5A shows pSTAT3 phosphorylation was induced by canine IL-31 in a dose-dependent manner with an optimal concentration of 1 μg/mL which was chosen to assess the phosphorylation level for assay assessment. FIG. 5B shows pSTAT3 was induced by canine IL-31 in a time-dependent manner with 5 minute as the time point chosen for the inhibition assays described in the following Examples to assess the inhibitory potency of anti-IL-31 antibodies.

### Example 5

### Inhibition of IL-31-Mediated pSTAT3 Signaling in a Cell-Based Assay

a. IL-31 Mediated DSTAT3 Signaling

DH-82 cells were plated into 96-well flat-bottomed cell culture plates at a density of 1×10⁵ cells per well in MEM growth media containing 15% heated-inactivated fetal bovine serum, 2 mmol/L GlutaMax, 1 mmol/L sodium pyruvate, 50 mg/L gentamicin and canine interferon-7 at 20 ng/mL for 16 hours at 37° C. in humidified air supplemented with 5% CO₂. Cells were serum starved for two hours prior to IL-31 treatment to increase IL-31 receptor expression. During starvation, 100 μl of the serial diluted antibody samples purified by protein A resin from guinea pig immune sera and canine IL-31 at 1 μg/mL were mixed well and co-incubated for 1 hour at 37° C. Following the pre-treatment, 50 μl recombinant canine IL-31 and purified sera sample mixtures were added for 5 minutes. After the inoculation, cells were lysed with 20% final volume of Cell Lysis Mix (5×), and shaken at 300 rpm at 37° C. for 10 min. 50 μl of cell lysates were used to evaluate pSTAT3. 50 μl of peroxidase-labeled anti-pSTAT3(y705) or peroxidase-labeled anti-STAT3 antibodies were added into testing wells and to react for 1 hour at 37° C. Then wells were washed with PBS containing 0.05% TWEEN 20 five times and reacted with 1000 of TMB substrate for another 15 minutes. Reactions were stopped by 2N H₂SO₄ and determined the absorbance at 450 nm.

b. Results

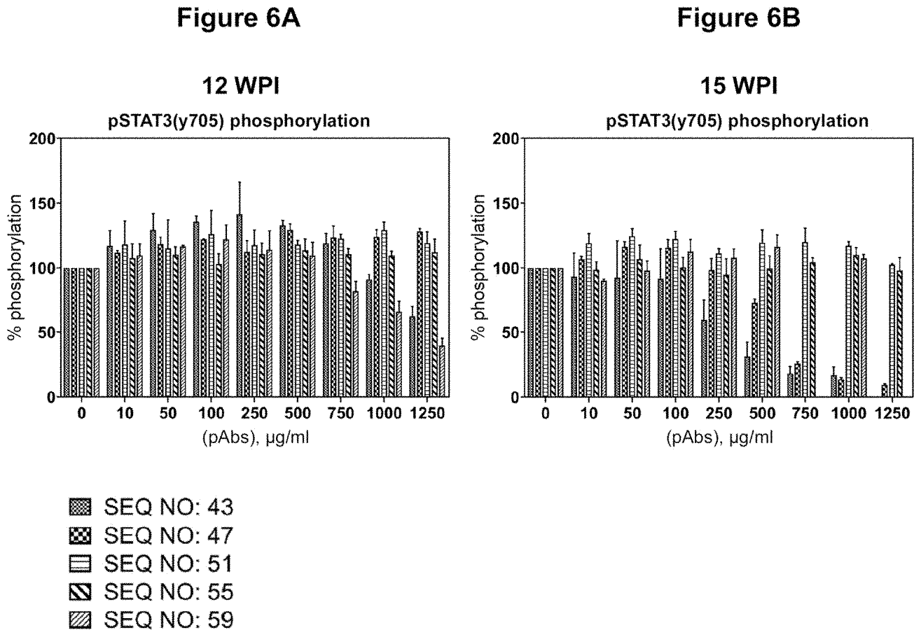

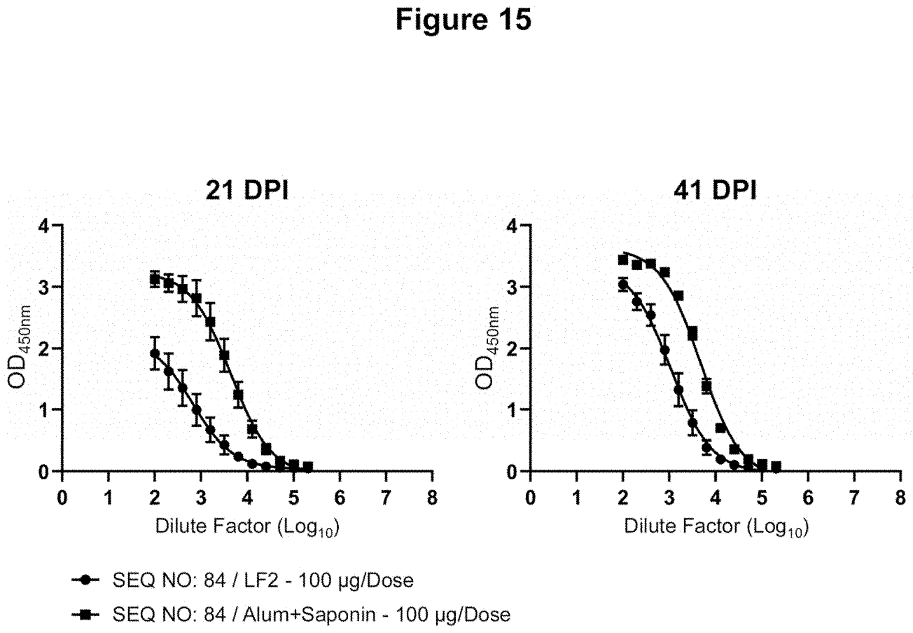

FIGS. 6A and 6B illustrates graphs showing the inhibition of canine IL-31 induced pSTAT3 signaling in canine DH82 monocytes by anti-IL-31 antibodies derived from the disclosed IL-31 peptide immunogen constructs (SEQ ID NOs: 43, 47, 51, 55 and 59) shown at 12 weeks post initial immunization (wpi) (FIG. 6A) and 15 wpi (FIG. 6B).

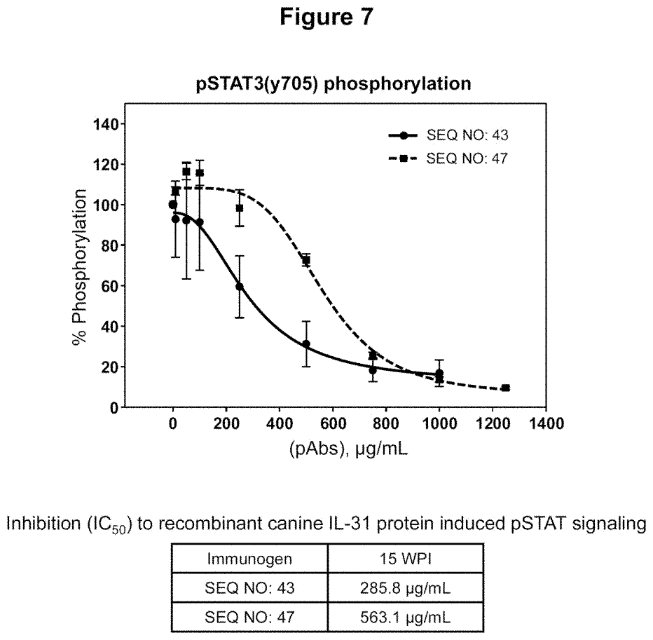

FIG. 7 contains a graph that shows additional details regarding the inhibition of canine IL-31 induced pSTAT3 signaling in canine DH82 monocytes by anti-IL-31 antibodies derived from IL-31 peptide immunogen constructs p4751 kb (SEQ ID NO: 43) and p4752 (SEQ ID NO: 47) at 15 weeks post initial immunization (wpi). FIG. 7 also reports the inhibition (IC₅₀) to recombinant canine IL-31 protein induced pSTAT signaling.

### Example 6

### Further Refinement of Canine IL-31 Peptide Immunogen Constructs for Immunogenicity Assessment in Guinea Pigs

a. Animals

A total of 18 guinea pigs (300-350 g by weight) were used for the immunization. The 18 guinea pigs were divided into 6 groups with 3 animals per group and an initial immunization with 400 μg of peptide (SEQ ID NOs: 63, 68, 71, 76, 80, and 84) followed by 4 boosters on 3. 6, 9 and 12 weeks post initial immunization (wpi), as shown in Table 7. Blood samples were collected at 0, 3, 6, 9, 12 and 15 wpi for titers of anti-IL-31 antibodies by their corresponding IL-31 peptide immunogen constructs.

b. Formulation

In general, the formulations used for the immunization contained the following:

1. 400 μg of IL-31 peptide immunogen construct

2. CpG at a peptide: CpG ratio of 0.7:1

3. 0.2% TWEEN 80

c. Titration of Serum Anti-IL-31 Antibodies by ELISA

The titration of serum anti-IL-31 antibodies elicited by the IL-31 peptide immunogen constructs were evaluated according to the following protocol:

Wells of 96 well plates were coated with 100 μl of canine IL-31 recombinant protein (50 ng/well) or the respective IL-31 B epitope peptide (0.2 μg/100 μL/well) for 1 hour at 37° C. at 1 μg/ml in 50 mM carbonated-bicarbonate buffer, pH9.6.

The rcIL-31 or the respective IL-31 B epitope peptide (SEQ ID NOs: 9 or 12) coated wells were incubated with 200 μl of 1% BSA in PBS at 37° C. for 1 hour to block non-specific protein binding, followed by three washes with PBS containing 0.05% TWEEN 20 and then dried. One hundred microliters (100 μl) of the serial diluted sera samples from immunized guinea pigs were added to each well and to react for 1 hour at 37° C. The wells were then washed with PBS containing 0.05% TWEEN 20 five times and dried. 100 μl of peroxidase-labeled goat anti-guinea pig IgG with optimal dilution prepared in PBS containing 1% BSA and 0.05% TWEEN 20 was added to each well and incubated at 37° C. for 1 hour. After the incubation, wells were washed with PBS containing 0.05% TWEEN 20 six times and reacted with 1001 of TMB substrate for another 15 minutes. Reactions were stopped by 2N H₂SO₄ and determined the absorbance at 450 nm.

For the determination of antibody titers in guinea pigs that received IL-31 peptide-based vaccine formulation, a 10-fold serial dilution of sera, from 1:100 or more, was performed for initial screening on the representative target “IL-31” B epitope peptides with titers expressed as Log₁₀. For further analysis of sera cross-reactive with recombinant canine IL-31, a 2-fold serial dilution of sera from 1:100 to 1:54,248,800 was performed on the ELISA and the titers expressed as Log₁₀ EC₅₀.

d. Results

Results from the immunogenicity assessment study are shown in Table 8.

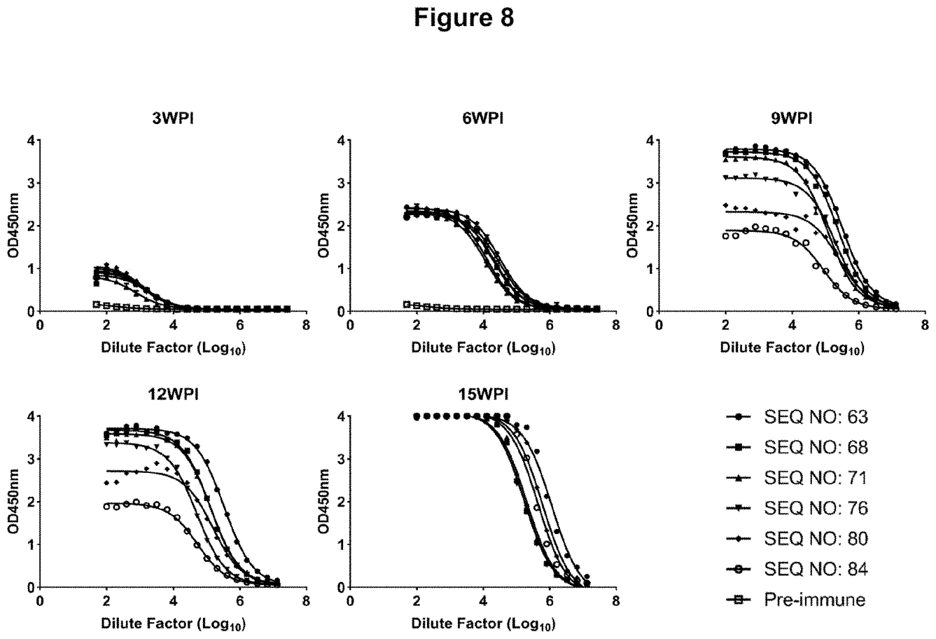

The antibody titers (Log EC₅₀) to recombinant IL-31 for each of the IL-31 peptide immunogen constructs (SEQ ID Nos: 63, 68, 71, 76, 80 and 84) are shown in Table 9 and FIG. 8.

High immunogenicity was found with these carefully designed canine IL-31 peptide immunogen constructs where high titer antibodies were elicited even upon one single administration at 3 wpi. High cross-reactivity towards canine rIL-31 was found with antibodies elicited by carefully designed IL-31 peptide immunogen constructs (SEQ ID NOs: 63, 68. 71, 76, 80 and 84).

### Example 7

### Inhibition of IL-31-Mediated pSTAT3 Signaling by Anti-IL-31 Antibodies from Guinea Pig Immune Sera in a Cell-Based Assay

a. IL-31 Mediated pSTAT3 Signaling

DH-82 cells were plated into 96-well flat-bottomed cell culture plates at a density of 1×10_{cells per well in MEM growth media containing} 15% heated-inactivated fetal bovine serum, 2 mmol/L GlutaMax, 1 mmol/L sodium pyruvate, 50 mg/L gentamicin and canine interferon-γ at 20 ng/mL for 16 hours at 37° C. in humidified air supplemented with 5% CO₂. Serum starved for two hours prior to IL-31 treatment to increase IL-31 receptor expression. At the meantime, 100p of the serial diluted sera samples purified by protein A resin from 6 and 12 WPI immunized guinea pigs and canine IL-31 at 1 μg/mL mixed well to co-incubated for 1 hour at 37° C. Following the pre-treatment, 50 μl recombinant canine IL-31 and purified sera sample mixtures were added for 5 minutes. After the inoculation, cells were lysed with 20% final volume of Cell Lysis Mix (5×), and shaken at 300 rpm at 37° C. for 10 min. 50 μl of cell lysates were used to evaluate pSTAT3. 50 μl of peroxidase-labeled anti-pSTAT3(y705) or peroxidase-labeled anti-STAT3 antibodies were added into testing wells and to react for 1 hour at 37° C. Then wells were washed with PBS containing 0.05% TWEEN 20 five times and reacted with 100 μl of TMB substrate for another 15 minutes. Reactions were stopped by 2N H₂SO₄ and determined the absorbance at 450 nm.

b. Results

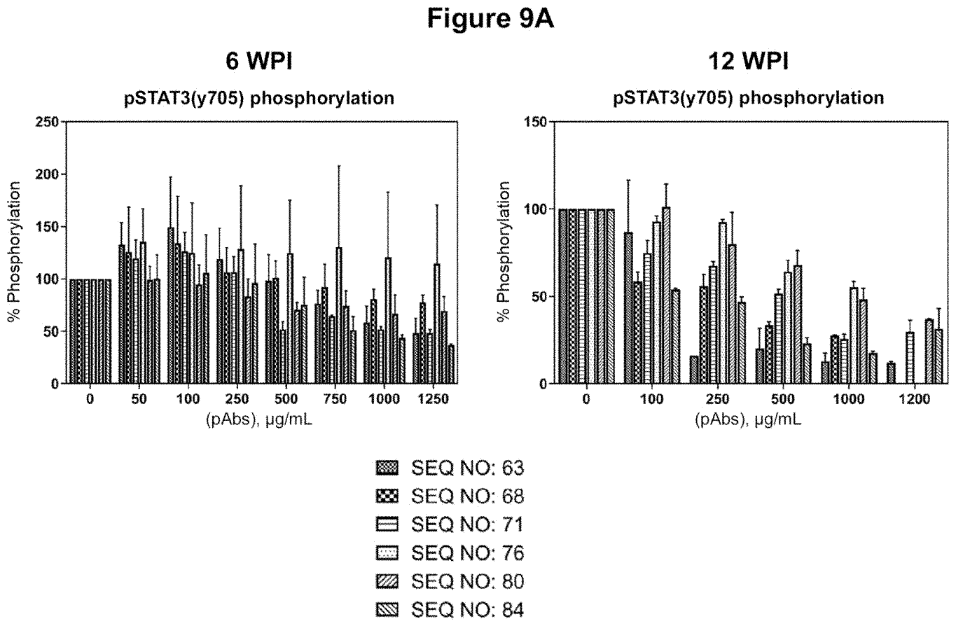

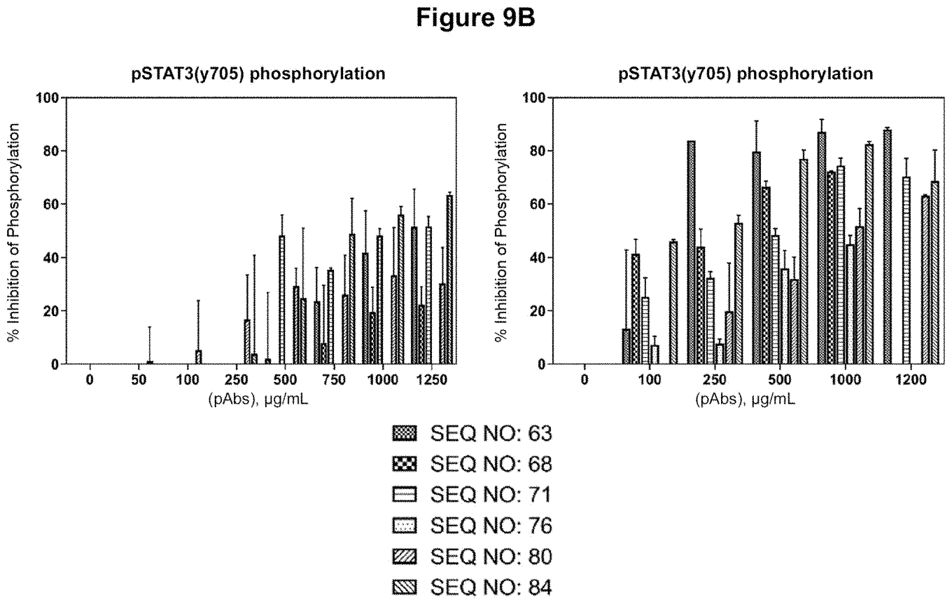

FIGS. 9A and 9B contain graphs showing the % phosphorylation (FIG. 9A) and inhibition of STAT3 phosphorylation (FIG. 9B) by canine IL-31 induced pSTAT3 signaling in canine DH82 monocytes in the presence of anti-IL-31 antibodies derived from guinea pig immune sera obtained with the disclosed IL-31 peptide immunogen constructs (SEQ ID NOs: 63, 68, 71, 76, 80 and 84) shown at 6 and 12 weeks post initial immunization (wpi) (FIG. 9A).

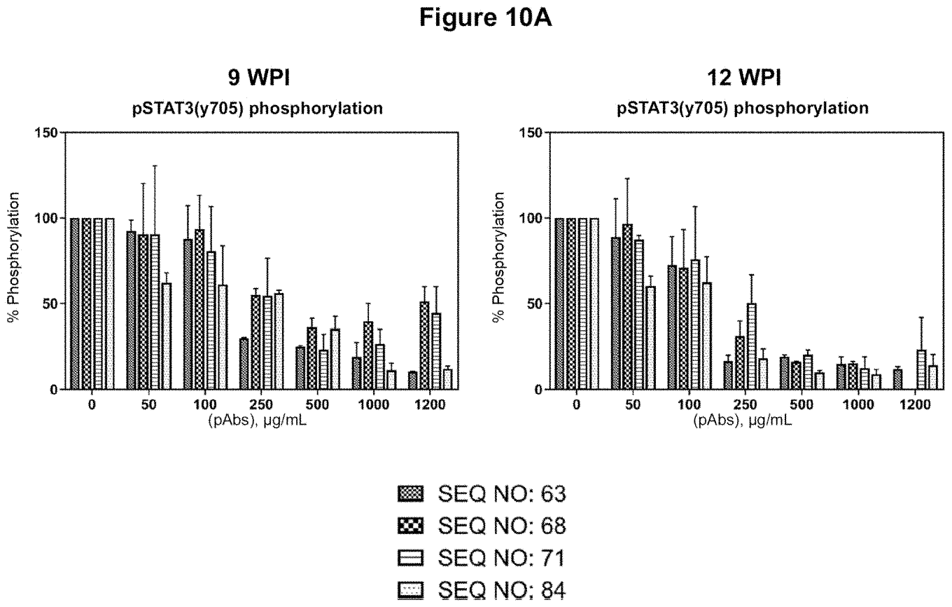

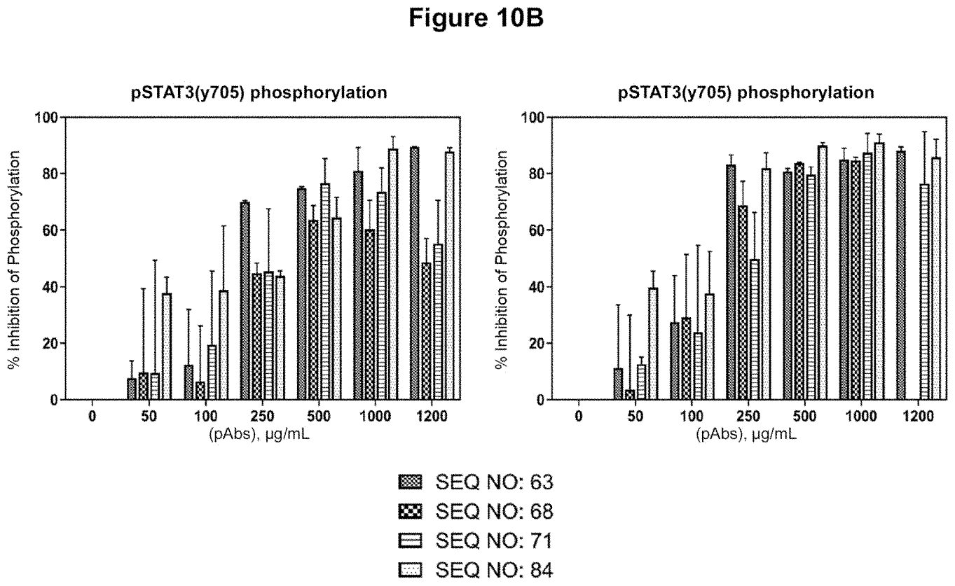

FIGS. 10A and 10B contain graphs showing the % phosphorylation (FIG. 10A) and inhibition of STAT3 phosphorylation (FIG. 10B) of canine IL-31 induced pSTAT3 signaling in canine DH82 monocytes by anti-IL-31 antibodies derived from guinea pig immune sera obtained with the disclosed IL-31 peptide immunogen constructs (SEQ ID NOs: 63, 68, 71, 84) shown at 9 and 12 weeks post initial immunization (wpi).

In general, significant inhibition of IL-31 induced pSTAT3 signaling in canine DH82 monocytes was found in a dose dependent manner for anti-L-31 reactive antibodies elicited by these carefully designed IL-31 peptide immunogen constructs.

### Example 8

### Inhibition of IL-31-Mediated pSTAT3 Signaling in a Cell-Based Assay

a. IL-31 Mediated pSTAT3 Signaling

DH-82 cells were plated into 96-well flat-bottomed cell culture plates at a density of 1×10⁵ cells per well in MEM growth media containing 15% heated-inactivated fetal bovine serum, 2 mmol/L GlutaMax, 1 mmol/L sodium pyruvate, 50 mg/L gentamicin and canine interferon-γ at 20 ng/mL for 16 hours at 37° C. in humidified air supplemented with 5% CO₂. Serum starved for two hours prior to IL-31 treatment to increase IL-31 receptor expression. At the meantime, 100 μl of the serial diluted sera samples purified by protein A resin from 6, 9, and 12 WPI immunized guinea pigs and canine IL-31 at 1 μg/mL mixed well to co-incubated for 1 hour at 37° C. Following the pre-treatment, 50 μl recombinant canine IL-31 and purified sera sample mixtures were added for 5 minutes. After the inoculation, cells were lysed with 20% final volume of Cell Lysis Mix (5×), and shaken at 300 rpm at 37° C. for 10 min. Fifty microliters (50 μl) of cell lysates were used to evaluate pSTAT3. Fifty microliters (50 μl) of peroxidase-labeled anti-pSTAT3(y705) or peroxidase-labeled anti-STAT3 antibodies were added into testing wells and to react for 1 hour at 37° C. Then wells were washed with PBS containing 0.05% TWEEN 20 five times and reacted with 100 μl of TMB substrate for another 15 minutes. Reactions were stopped by 2N H₂SO₄ and determined the absorbance at 450 nm.

b. Results

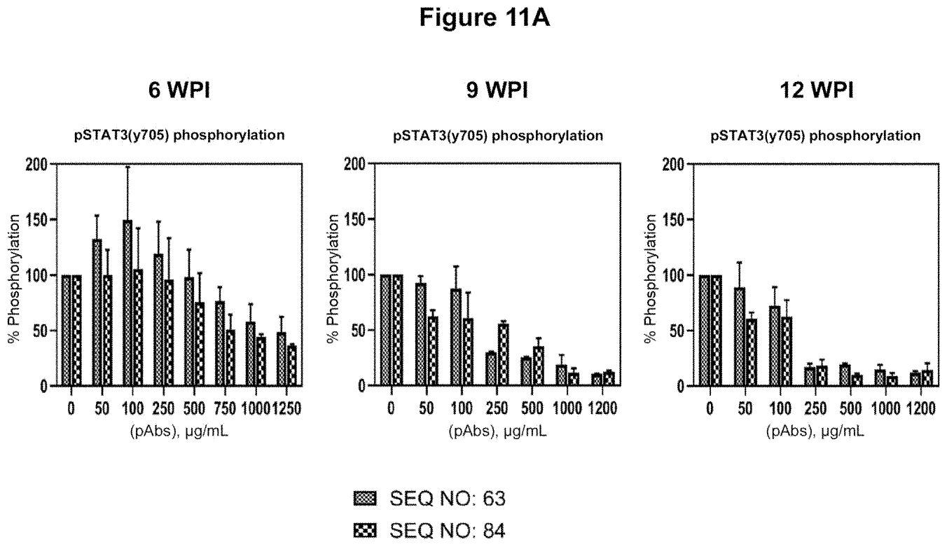

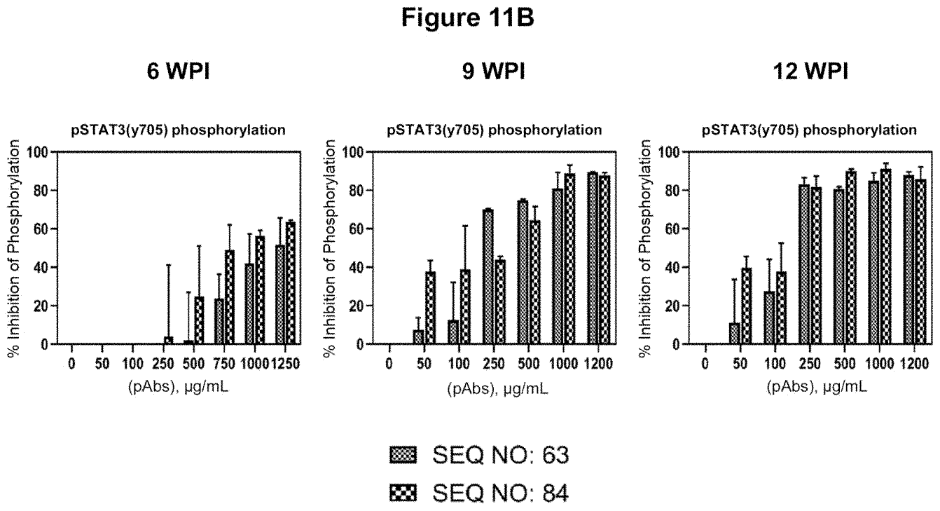

FIGS. 11A and 11B contain bar graphs showing the percent phosphorylation (FIG. 11A) and percent inhibition (FIG. 11B) of canine IL-31 induced pSTAT3 signaling in canine DH82 monocytes in the presence of anti-IL-31 antibodies derived from the guinea pig immune sera obtained with IL-31 peptide immunogen constructs p4854 kb (SEQ ID NO: 63) and p4859 (SEQ ID NO: 84) at 6, 9, and 12 weeks post initial immunization (wpi).

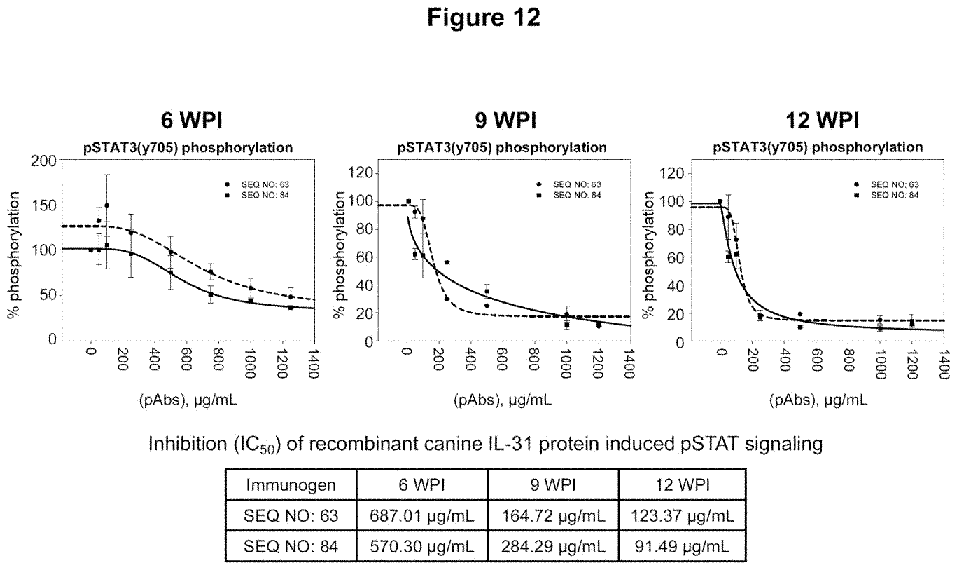

FIG. 12 illustrates graphs in curve fitting showing the percent phosphorylation of canine IL-31 induced pSTAT3 signaling in canine DH82 monocytes by anti-IL-31 antibodies derived from the guinea pig immune sera obtained with IL-31 peptide immunogen constructs p4854 kb (SEQ ID NO: 63) and p4859 (SEQ ID NO: 84) at 6, 9, and 12 weeks post initial immunization (wpi). The bottom panel reports the corresponding inhibition (IC₅₀) to recombinant canine IL-31 protein induced pSTAT signaling.

In general, significant inhibition of IL-31 induced pSTAT3 signaling in canine DH82 monocytes was found in a dose dependent manner for anti-IL-31 reactive antibodies elicited by these carefully designed IL-31 peptide immunogen constructs.

### Example 9

### Production of Lipidated Proteins in E. Coli

The IL-31 peptide immunogen constructs can be expressed in specific E. coli strains which allow for lipidation to make a lipoprotein and used as an immunogen for treatment of atopic dermatitis.

This process is described in U.S. Pat. No. 8,426,163, which is incorporated herein by reference in its entirety.

### Example 10

### Immunization of Dogs with a Selected IL-31 Peptide Immunogen Construct (Seq Id No: 84) for Functional Immunogenicity Assessment in a Proof of Concept Trial

a. Animals

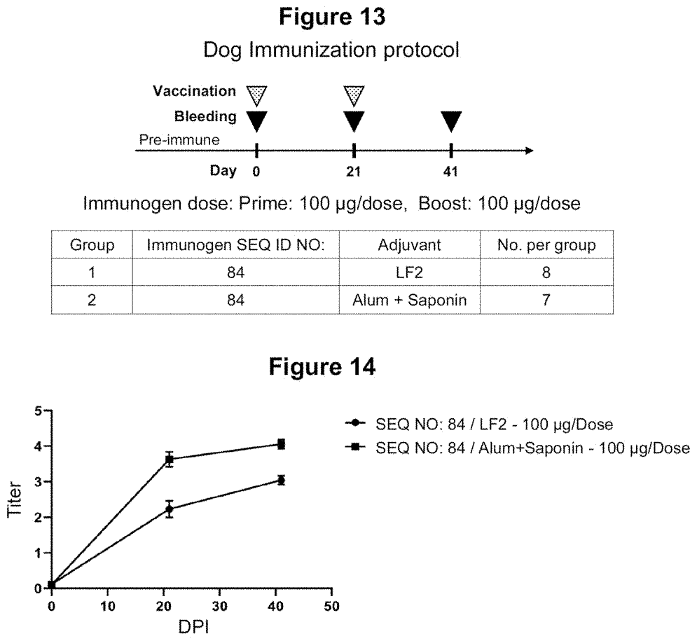

A total of 15 beagle dogs were used for the immunization. The dogs were divided into 2 groups, one group of 8 dogs was tested with water-in-oil emulsion formulation and the other group of 7 dogs was tested for alum combined with saponin formulation. An initial immunization with 100 μg of IL-31 peptide immunogen construct with SEQ ID NO: 84 was followed by another boost with the same formulation at the same dose on 21 days post initial immunization (DPI). Blood samples were collected at 0, 21 and 41 DPI for measurement of titers of anti-IL-31 antibodies and for assessment of other functional properties of the antibodies present in the corresponding immune sera. The immunization protocol is shown in FIG. 13.

b. Titration of Serum Anti-IL-31 Antibodies to IL-31 B Epitope Peptide or Recombinant Canine IL-31 Protein by ELISA

The titration of serum anti-IL-31 antibodies elicited by the IL-31 peptide immunogen construct (SEQ ID NO: 84) were evaluated according to the following protocol:

Wells of 96 well plates were coated with 100 μl of canine IL-31 recombinant protein (50 ng/well) or IL-31 B epitope peptide (SEQ ID NO:84) at 0.2 μg/0.1 mL/well for 1 hour at 37° C. at 1 μg/ml in 50 mM carbonated-bicarbonate buffer, pH9.6. The antigen coated wells were incubated with 200 μl of 1% BSA in PBS at 37° C. for 1 hour to block non-specific protein binding, followed by three washes with PBS containing 0.05% TWEEN 20 and then dried. One hundred microliters (100 μl) of the serial diluted sera samples from immunized dogs were added to each well and to react for 1 hour at 37° C. The wells were then washed with PBS containing 0.05% TWEEN 20 five times and dried. One hundred microliters (100 μl) of peroxidase-labeled rabbit anti-dog IgG or peroxidase-labeled recombinant protein A/G (Pierce™) with optimal dilution prepared in PBS containing 1% BSA and 0.05% TWEEN 20 was added to each well and incubated at 37° C. for 1 hour. After the incubation, wells were washed with PBS containing 0.05% TWEEN 20 six times and reacted with 100 μl of TMB substrate for another 15 minutes. Reactions were stopped by 2N H₂SO₄ and determined the absorbance at 450 nm. For the determination of antibodies titers in Beagle dogs that received IL-31 peptide-based vaccine formulation, a 2-fold serial dilution of sera from 1:100 to 1:54,248,800 was performed on the ELISA and the titers expressed as Log₁₀.

c. Inhibition of IL-31-Mediated pSTAT3 Signaling in a Cell-Based Assay

DH-82 cells were plated into 96-well flat-bottomed cell culture plates at a density of 1×10⁵ cells per well in MEM growth media containing 15% heated-inactivated fetal bovine serum, 2 mmol/L GlutaMax, 1 mmol/L sodium pyruvate, 50 μg/L gentamicin and canine interferon-γ at 20 ng/mL for 16 hours at 37° C. in humidified air supplemented with 5% CO₂. Serum starved for two hours prior to IL-31 treatment to increase IL-31 receptor expression. During starvation, 100 μl of the serially diluted sera samples purified by protein A resin from immunized beagles and canine IL-31 at 1 μg/mL mixed well to co-incubated for 1 hour at 37° C. Following the pre-treatment, 50 μl recombinant canine IL-31 and purified sera sample mixtures were added for 5 minutes. After the inoculation, STAT3 phosphorylation was determined with a commercial kit (InstantOne™ ELISA Kit, Thermo). Cells lysed with 20% final volume of Cell Lysis Mix (5×) and shaked at 300 rpm at 37° C. for 10 min. 50 μl of cell lysates were used to evaluated pSTAT3 and prepared 50 μl of peroxidase-labeled anti-pSTAT3(y705) or peroxidase-labeled anti-STAT3 antibodies were added into testing wells and to react for 1 hour at 37° C. Then wells were washed with PBS containing 0.05% TWEEN 20 five times and reacted with 100 μl of TMB substrate for another 15 minutes. Reactions were stopped by 2N H₂SO₄ and determined the absorbance at 450 nm.

d. Results

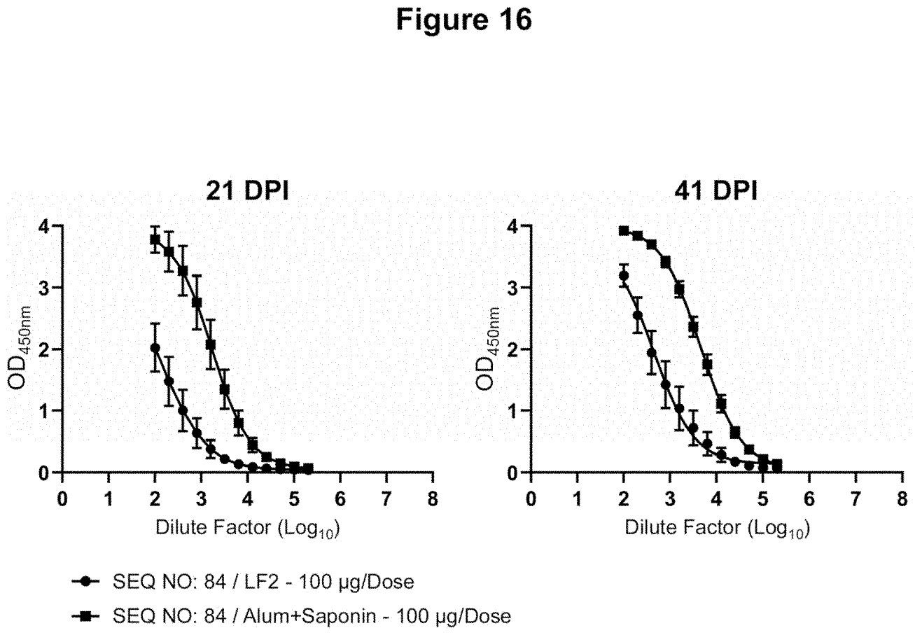

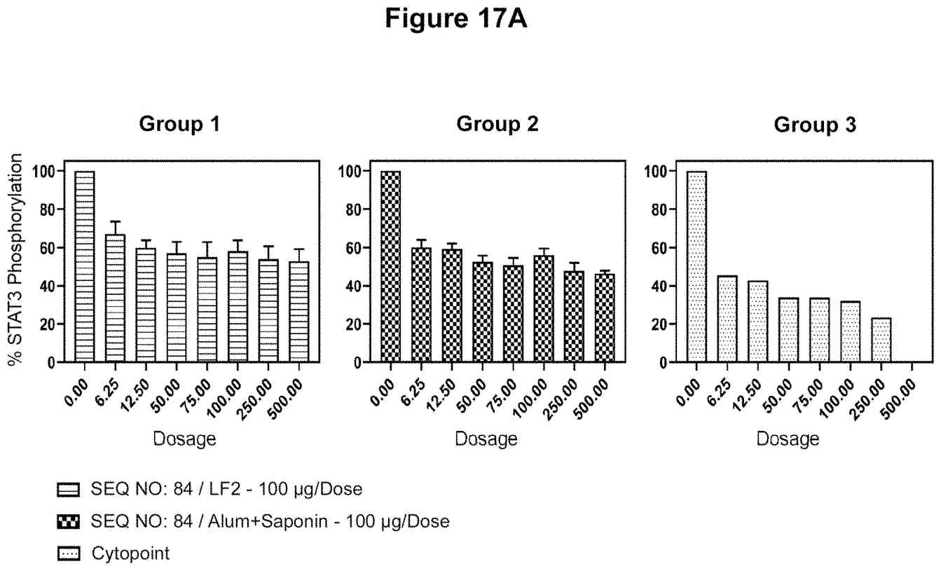

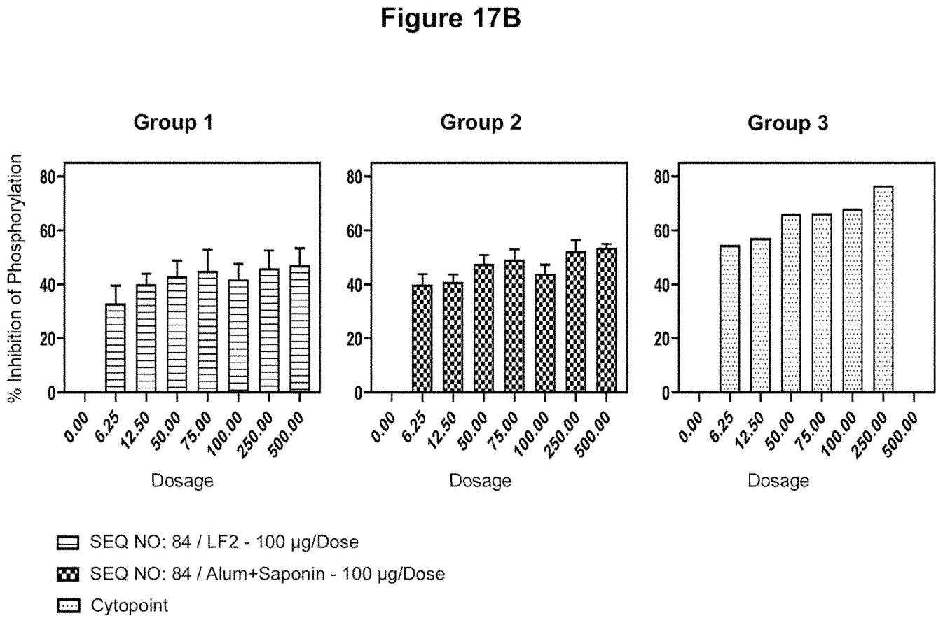

The results from the immunogenicity assessment by different formulations with respective titers against the target IL-31 B epitope peptide (SEQ ID NO: 84) are shown in FIG. 14. The antibody titers (Log₁₀ EC₅₀) to recombinant canine L-31 on 21 DPI and 41 DPI for each of the dogs are shown in FIGS. 15 and 16, where rabbit anti-dog IgG HRP (FIG. 15) or protein A/G HRP (FIG. 16) was used as the second tracer.

High immunogenicity was found with a representative canine IL-31 peptide immunogen construct with SEQ ID NO:84 where high titer antibodies were elicited upon one single administration at 21 DPI towards the IL-31B epitope peptide (FIG. 14) and with high cross-reactivity towards canine rIL-31 (FIGS. 15 and 16).

In this formulation study, it is clear that alum in the presence of adjuvant Saponin gave better immune boosting than the water in oil emulsion formulation. Both tracers gave similar binding profiles and comparable titers. Another significant finding of this immunogenicity study in dogs is that the proprietary UBITh®-T helper peptide was able to assist the self-IL-31 sequence to breakout/or overcome the immune tolerance inherent in most of self-proteins.

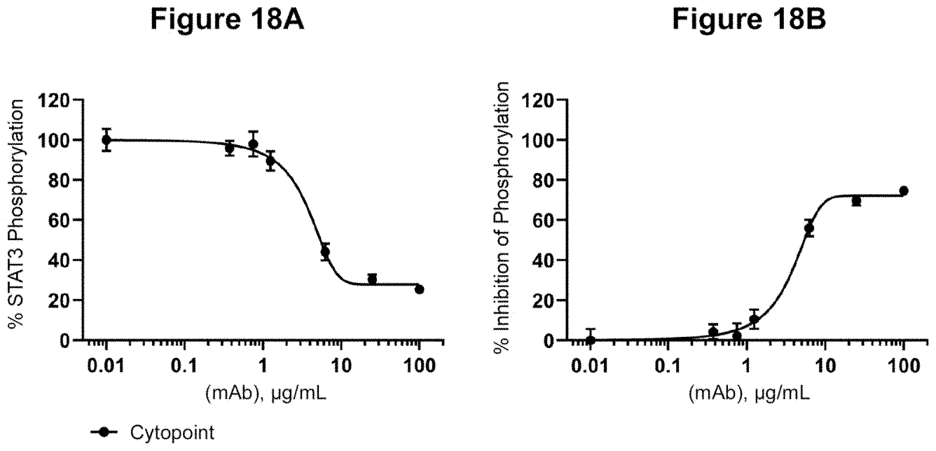

In general, significant inhibition of IL-31 induced pSTAT3 signaling in canine DH82 monocytes was found in a dose dependent manner (from 500, 250, 100, 75, 50, 12.5, to 6.25 ug) for canine anti-canine IL-31 reactive antibodies elicited by this carefully designed IL-31 peptide immunogen construct for both formulations with alum/saponin formulation giving about 40-60% inhibition of IL-31-induced pSTAT3 signaling phosphorylation, with emulsion formulation giving about 35 to 50% inhibition in comparison to a monoclonal anti-IL-31(Cytopoint) was giving comparably 50 to 75% inhibition. As shown in FIGS. 18A and 18B, monoclonal antibody to canine IL-31 “Cytopoint” has a potency in signaling inhibition of IC₅₀=3.21 μg/mL.

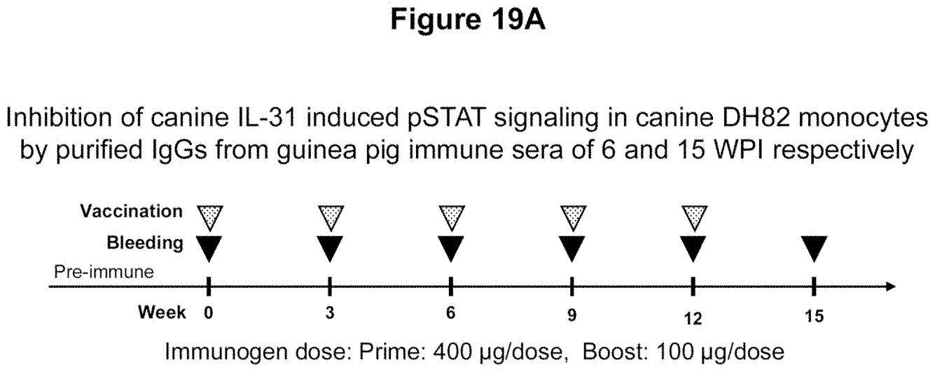

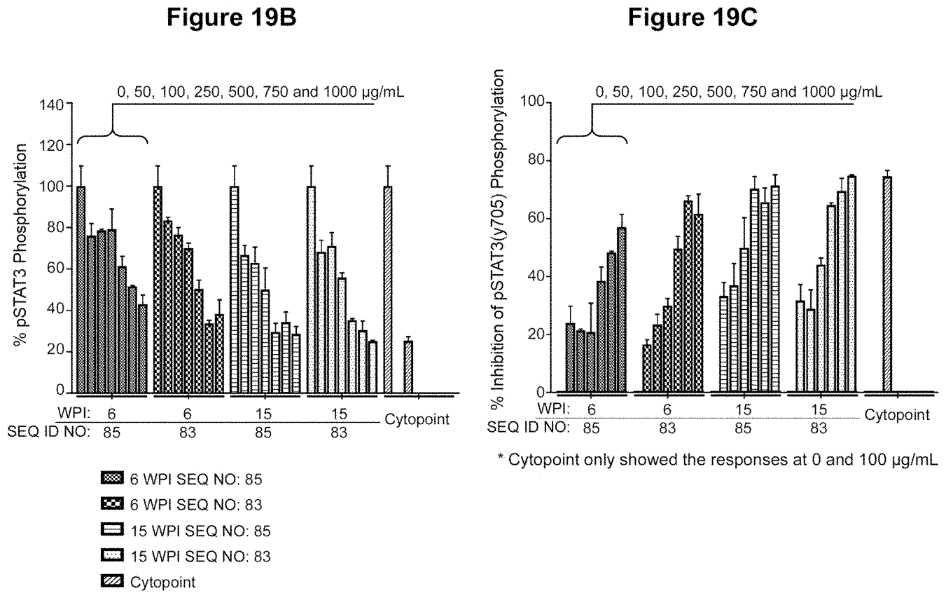

In a related guinea pig immunogenicity study, the antibodies from 6 wpi immune sera after 2 immunizations has a far lower % inhibition in such pSTAT3 signaling where antibodies from 15 wpi immune sera gave a higher dose dependent overall % inhibition of signaling (see FIGS. 19A to 19C). These data suggest that immunization of dogs with more than 2 injections provide even better signaling inhibition. Such canine IL-31 peptide immunogen construct thus has demonstrated its potential and ability to induce in dogs effective polyclonal antibodies to reduce/suppress/inhibit IL-31's stimulatory effect which could cause atopic dermatitis in dogs.

### Example 11

### Further Refinement in the Design of Human IL-31 Peptide Immunogen Constructs for Functional Immunogenicity Assessment in Guinea Pigs

a. Design of Human IL-31 Related Peptide Immunogen Constructs as an Extension to the Canine IL-31 Analogues

IL-31 is a four-helix bundle cytokine belonging to the IL-6 cytokine family. IL-31 is preferentially produced by activated CD4+TH2 cells, and by cutaneous lymphocyte-associated antigen-positive skin-homing CD45RO+(memory) T cells. IL-4 induces the gene expression and release of IL-31 protein from human TH2 cells and IL-33 further potentiates the IL-4-induced IL-31 release.

The IL-31 receptor (IL-31R) consists of a heterodimer of IL-31 receptor α chain (IL-31Rα) and oncostatin M receptor β (OSMR β). The heterodimeric IL-31R is expressed in macrophages, dendritic cells, basophils, cutaneous neurons and epithelial cells, including keratinocytes. IL-31Rα is most abundantly expressed in dorsal root ganglia, where cell bodies of cutaneous sensory neurons are located. Recent studies suggest IL-31 might communicate directly with sensory nerves through IL-31R and serve as a critical neuroimmune link between Th2 cells and sensory neurons for the generation of T cell-mediated inflammatory itch. OSMR β not only is a subunit of IL-31R but also interacts with gp130 to form type II receptor complex-binding OSM. OSMR β is widely expressed in the vascular system, heart, lung, adipose tissue, skin, bladder, mammary tissue, adrenal gland, and prostate.

IL-31 binds predominantly to IL-31Rα, but not to OSMR β, in the IL-31R complex. However, on coupling, OSMR β converts IL-31R into a high-affinity receptor and increases IL-31 binding. Interaction of IL-31 and the IL-31Rα/OSMR β receptor complex induces activation of the Jak/signal transducer and activator of transcription (STAT), phosphoinositide 3-kinase (PI3K)/AKT signal-transducing and mitogen-activated protein kinase (MAPK) pathways.

X-ray structural data for IL-31 or the IL-31/L-31Rα complex are not available yet. However, site-directed mutagenesis suggests the interaction of site II of human IL-31 with IL-31Rα and site III with OSMR β. In particular, E22 in helix A and E83/H87 in helix C form the site II and KI 11 in the N-terminus of helix D is a crucial part of site III. Ab initio modeling of human IL-31 generated by 1-TASSER showed a classical four-helix bundle fold of the IL-31 cytokine family with an up-up down-down topology. Three key residues, E22, E83 and H87, for IL-31Rα interaction are spatially clustered, exhibiting a predicted site II.

The human IL-31 B cell epitopes of the IL-31 peptide immunogen constructs were further designed to target the IL-31Rα binding region on the IL-31 molecule. To maintain the conformation of side chains of E83 and H87 being exposed to solvent, helix C was constrained by neighboring helixes in three different ways: (1) helix C-loop-helix D (SEQ ID NO: 13), (2) helix B-loop-helix C (SEQ ID NO:95), and (3) helix B-C-D (SEQ ID NO: 93). The B cell epitope peptides from 31 to 64 amino acid residues derived from L75-S122, S62-I92 or A64-L127. To make the side chains of E83 and H87 face in the same direction, the helix C epitope within p5095 kb (SEQ ID NO: 101) was constrained by helix B through helix-helix interaction and introduction of an artificial disulfide bond. A peptide construct p5094 kb (SEQ ID NO: 100) with mere helix C as B epitope (SEQ ID NO: 94) was also tested.

b. IL-31 Based ELISA Test for Antibody Specificity Analysis

Recombinant humanIL-31 protein (Sino Biological Inx.) or individual human IL-31B epitope peptides (SEQ ID NOs: 13, 94, 95 and 93) was immobilized on microtitre plates at 50 ng/well for recombinant human IL-31 protein or for respective IL-31 B epitope peptide at 200 ng/well in coating buffer (1.5 g/L Na₂CO₃, 2.9 g/L NaHCO₃, pH 9.6) and incubated at 4° C. overnight. Plates were washed 3 times with 200 μL/well of wash buffer (PBS with 0.05% TWEEN-20) Coated wells were blocked by 200 μL/well of assay diluent (0.5% BSA, 0.05% TWEEN-20, 0.01% ProClin 300 in PBS) at room temperature for 1 hour. Plates were washed 3 times with 200 μL/well of wash buffer, 100 μL of antiserum (serially 4-fold diluted from 1:100 to 1:4.19×1R, total 12 dilutions) or antibody diluents (serially 4-fold diluted from 100 mg/mL to 0.0238 ng/mL, total 12 dilutions) were added to coated wells. The incubation was carried out at room temperature for 1 hour. All wells were aspirated and washed 5 times with 200 μL/well of wash buffer. The plates were incubated with a 1:10,000 dilution of HRP-conjugated goat anti-guinea pig IgG (H+L) antibody (Jackson ImmunoResearch Laboratories) for 1 hour (100 μL/well). Then all wells were washed 5 times with 200 μL/well of wash buffer. Finally, wells were developed by 100 μL/well of NeA-Blue TMB substrate (Clinical Science Products) and the reaction was stopped by addition of 100 μL/well of 1M H₂SO₄. The absorbance was measured at OD₄₅₀ on an ELISA microplate reader (Molecule Devices). The specific titer of antiserum, expressed as Log₁₀ Titers or Log₁₀(EC₅₀), was determined as the sample dilution in log giving 50% of the maximum absorbance to a four-parameter curve by using nonlinear regression analysis in a Prism software (GraphPad). The reactivity of purified polyclonal IgG antibody was expressed as half of effective concentration (EC₅₀) to a four-parameter curve by using nonlinear regression analysis using Prism software.

c. Assessment of Functional Properties of Antibodies Elicited by the IL-31 Peptide Immunogen Constructs and Formulations Thereof in Animals

Immune sera or purified anti-IL-31 polyclonal antibodies in immunized vaccines were further tested for their ability to (1) block the interaction between IL-31 and its receptor IL-31Rα, (2) suppress the IL-31-induced STAT3 phosphorylation in U87MG glioma cells and (3) inhibit IL-20 expression in a HaCaT cell line transfected with IL-31Rα.

### 1. Cells

U87MG cell line was maintained in EMEM supplemented with 10% calf serum and 1% penicillin/streptomycin in a humidified 37° C. incubator with 5% CO₂.

IL-31Rα overexpressing HaCaT transfectants were maintained in DMEM medium (high glucose, Gibco) supplemented with 10% FBS, 1% penicillin/streptomycin, 1 mM sodium pyruvate and 200 μg/mL Hygromycin B (Gibco) in a humidified 37° C. incubator with 5% CO₂.

### 2. Binding of IL-31 to IL-31Rα Chain

The purified IgG polyclonal antibodies from pooled immune sera of guinea pigs previously immunized with different IL-31 peptide immunogen constructs were examined for their relative ability to inhibit the binding of IL-31 to IL-31Rα by ELISA. The wells of 96-well plates were coated individually with 50 ng of anti-His monoclonal antibody (GenScript) in coating buffer (15 mM Na₂CO₃, 35 mM NaHCO₃, pH 9.6) and incubated at 4° C. overnight. Coated wells were blocked by 200 μL/well of assay diluents (1% BSA, 0.05% TWEEN-20 and 0.01% ProClin 300 in PBS) at room temperature for 1 hour. Plates were washed 3 times with 200 μL/well of wash buffer (PBS with 0.05% TWEEN-20 and 0.01% ProClin 300), 100 ng of recombinant His-tagged human IL-31Rα protein (R&D systems) was immobilized on the anti-His surface at room temperature for 1 hour. After washing, 100 μL mixture of human IL-31 (GenScript) at 10 ng/mL and purified guinea pig IgG polyclonal antibodies at different concentrations was pre-incubated for 1 hour at room temperature and then added to coated wells. The incubation was carried out at room temperature for 1 hour. All wells were aspirated and washed 3 times with 200 μL/well of wash buffer. The captured IL-31 was detected by 100 μL/well of biotin-labeled rabbit anti-IL-31 antibody at 0.25 μg/mL (PeproTech Inc.) at room temperature for 1 hour. Then, the bound biotin-labeled antibodies were detected using streptavidin poly-HRP (1:10,000 dilution, Thermo Fisher Scientific) for 1 hour (100 μL/well). All wells were aspirated and washed 3 times with 200 μL/well of wash buffer. Finally, wells were developed by 100 μL/well of OptEIA TMB substrate (BD Biosciences) and the reaction was stopped by addition of 100 μL/well of IM H₂SO₄. The colorimetric absorbance was measured by a VersaMax ELISA Microplate Reader (Molecular Devices) and the reactivity curve was generated by using four parameter logistic curve-fitting for calculation of the half of maximal inhibitory concentration (IC₅₀) in Prism 6 software (GraphPad Software).

### 3. IL-31-Induced STAT3 Phosphorylation Assay

To investigate whether the purified IgG could inhibit IL-31-induced STAT3 phosphorylation in U87MG cells, cells were seeded in 12-well plate (2×10⁵/well) and starved in reduced-serum medium (1% calf serum in EMEM) for 16 hours. Cells were then simultaneously incubated with IL-31 at a final concentration of 10 ng/mL in the presence of guinea pig polyclonal antibodies at a in a total volume of 500 μL of reduced-serum medium at 37° C., 5% CO₂ for 30 min. An anti-IL-31 monoclonal antibody, MT313 (Mabtech AB), was also included as study control. The phosphorylated STAT3 level was measured by PathScan p-Stat3 ELISA kit (Cell Signaling). Briefly, the cell lysate was prepared by suspending cells in 30 μL of cell lysis buffer (Cell Signaling) supplied with 1% Phosphatase Inhibitor Cocktail 3 (Sigma-Aldrich) with cell debris removed by centrifugation at 12,000×g at 4° C. for 10 min. Ten micrograms (10 μg) of clear cell lysate was used to measure the content of phosphorylated STAT3 according to vendor's instructional brochure. The colorimetric absorbance was measured by a VersaMax ELISA Microplate Reader (Molecular Devices).

### 4. IL-31-Induced IL-20 Expression

HaCaT is a spontaneously transformed aneuploid immortal keratinocyte cell line from adult human skin. To facilitate examination of IL-31-induced IL-20 expression, a stable HaCaT clone that overexpressed human IL-31Rα was prepared, IL-31-dependent IL-20 expression could be modulated by anti-IL-31 antibodies elicited by the IL-31 peptide immunogen constructs of the present disclosure. The assay were performed by incubating 4×10⁵ cells, human recombinant IL-31 at a final concentration of 10 ng/mL and purified guinea pig IgG polyclonal antibodies at different concentrations in a total volume of 1,000 μL of culture medium per well at 37° C., 5% CO₂ for 1 hour. An anti-IL-31 monoclonal antibody. MT313 (Mabtech AB), was also included as study control. RNA was extracted by PureLink RNA Mini Kit (Thermo Fisher Scientific Inc.), according to the manufacturer's instruction, and residual genomic DNA was removed by SuperScript™ III/RNaseOUT Enzyme Mix (Thermo Fisher Scientific Inc.). A total of 1 μg of RNA was reverse transcribed back to cDNA by SuperScript III First-Strand Synthesis SuperMix kit (Thermo Fisher Scientific Inc.) and the resulting cDNA was analyzed by quantitative real-time PCR by using the Applied Biosystems 7500 Real-Time PCR Systems (Thermo Fisher Scientific Inc.). The real-time PCR reactions were performed with the Power SYBRGreen PCR Master Mix kit (Thermo Fisher Scientific Inc.). The QuantiTect primer pair for IL-20 (Hs_IL20_1_SG) was purchased from Qiagen and the primer pair for HPRT (forward: 5-TGACACTGGCAAAACAATGCA-3′ (SEQ ID NO: 106); reverse: 5′-GGTCCTTFTCACCAGCAAGCT-3′ (SEQ ID NO: 107)) was synthesized. All measurements were performed in duplicate. The relative quantification was calculated by using the comparative cycle threshold method and normalized to HPRT. The relative IL-20 expression level of each antibody-treated group was normalized to that of the untreated group.

d. Results

Design, screening, identification, assessment of functional properties and optimization of multi-component vaccine formulations incorporating representative human IL-31 peptide immunogen constructs

### 1. Selection of Human IL-31 B Cell Epitope Peptide Sequences

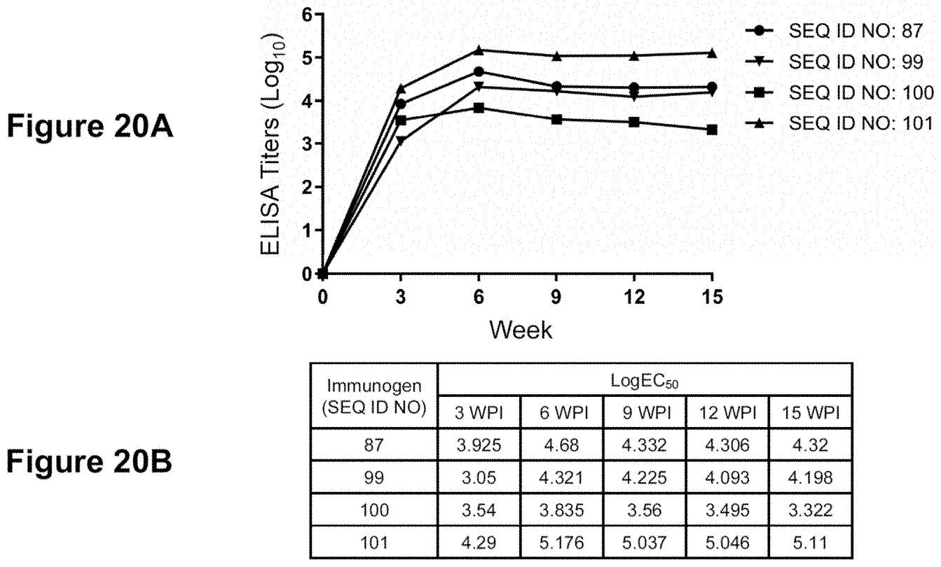

The helix C of IL-31 was selected for B cell epitope peptide design because two key residues, E83 and H87, in this region have been found to interact with IL-31Rα. The helix C-based peptide constructs, linked with UBITh1 T helper peptide (SEQ ID NO: 27) and a short linker εK, were formulated with ISA 51 and CpG for prime immunization in guinea pigs at 400 μg/1 mL and boosts (3, 6 and 9 wpi) at 100 μg/0.25 mL. To test the immunogenicity in guinea pigs, ELISA assay were used with guinea pig immune sera diluted at a 4-fold serial dilution from 1:100 to 1:4.19×10⁸. ELISA plates were coated with a recombinant human IL-31 protein at 50 ng per well. The titer of a tested serum, expressed as Log EC₅₀, was calculated by four-parameter nonlinear regression analysis of the A450 nm. The ELISA results showed the four peptide immunogen constructs not only induced high immunogenicity titers against the corresponding IL-31 B epitope peptide (Table 10 (SEQ ID NO: 87): Table 11 (SEQ ID NOs: 100 and 101; and Table 13 group 3 (SEQ ID NO: 99) and induced moderate-to-high cross reactivity against human IL-31 protein (FIGS. 20A and 20B).

2. Assessment of Immunogenicity of Human IL-31 Peptide Immunogen Constructs for their Antibodies to Inhibit IL-31 and IL-31Rα Interaction

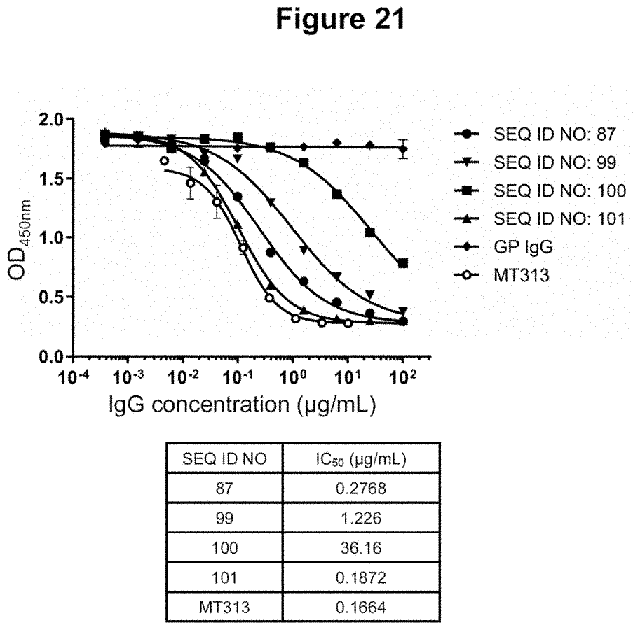

A study was performed to determine whether the antibodies from IL-31 peptide immunogen constructs generated in guinea pigs could neutralize IL-31 so as to block the interaction between IL-31 and IL-31Rα. Specifically, purified guinea pig IgGs from immune sera of guinea pigs immunized by 4 respective candidate IL-31 peptide immunogen constructs (SEQ ID NOs: 87, 99, 100, and 101) were employed in an ELISA assay, of which recombinant human IL-31Rα protein was immobilized on a solid phase coated with anti-His antibody. As shown in FIG. 21, representative antibodies purified from immune sera of guinea pigs immunized with respective IL-31 peptide immunogen constructs inhibited competitively in a dose dependent manner (from IOE-3 to 10E2 μg/mL) the IL-31 and IL-31Rα interaction.

### 3. Suppression of IL-31-Induced STAT3 Phosphorylation by Anti-IL-31 Antibodies

IL-31 signaling pathway is involved in the complex formation of L-31Rα/OSMRβ initially on cell membrane followed by the downstream protein STAT3 phosphorylation in cytoplasm. The U87MG cell line was used to assess the ability of those purified anti-L-31 antibodies derived from immune sera of guinea pigs immunized with IL-31 peptide immunogen constructs for their ability to suppress IL-31-induced STAT3 phosphorylation.

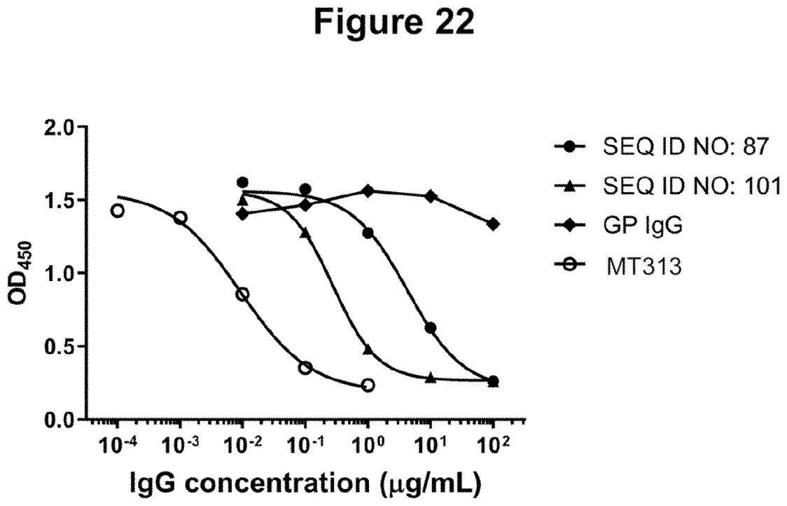

Firstly, cultured cells were treated with IL-31(10 ng/mL) and the purified IgGs at different concentrations simultaneously. The anti-IL-31 monoclonal antibody, MT313, was included as a positive control. As seen in FIG. 22, the anti-IL-31 IgGs elicited by representative immunogens (SEQ ID NOs: 87 and 101) could, along with MT313, reduce STAT3 phosphorylation in a dose dependent manner.

### 4. Suppression of IL-31-Induced IL-20 Expression

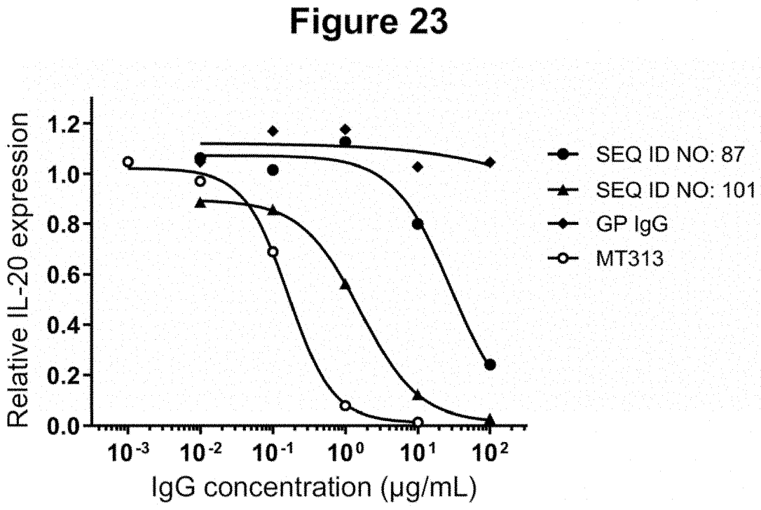

IL-20 is structurally related to IL-10 and an autocrine factor for keratinocytes that regulates their participation in inflammation. IL-31 can induce IL-20 expression in the keratinocytic cell line HaCaT. To investigate whether anti-L-31 antibodies elicited in guinea pigs by the IL-31 peptide immunogen constructs could suppress IL-31-dependent IL-20 expression in IL-31Rα-overexpressing HaCaT transfectants, all cell culture groups were treated with IL-31 at a concentration of 10 ng/mL for 30 min for induction of IL-20 gene transcription. Representative preparations of purified IgGs from immune sera of guinea pigs elicited by candidate IL-31 peptide immunogen constructs (SEQ ID NOs: 87 and 101) were added in the test groups at different concentrations and MT313 was also included as a positive control. The cell culture in the presence of IL-31 only without adding antibody was set up as a negative control. As shown in FIG. 23, an antibody concentration-dependent suppression of IL-20 expression was observed in the treatment groups with purified IgG antibodies elicited by representative candidate peptide constructs with SEQ ID NOs of 87 and 101.

The above ex-vivo functional studies suggest that an IL-31 peptide immunogen consisting of helix C constrained by helix B through helix-helix interaction and introduction of an artificial disulfide bond demonstrated the blockage of IL-31-IL-31Rα interaction and the suppression of IL-31-induced signaling cascade.

### 5. Assessment of Functional Immunogenicity of a Representative Human IL-31 Peptide Construct (SEQ ID NO: 101) in Various Multi-Component Formulations

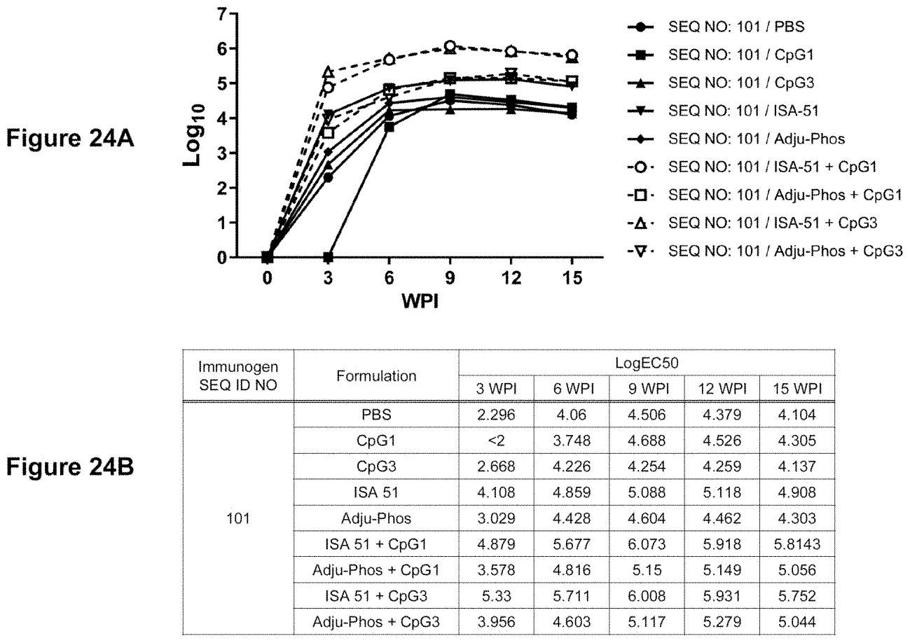

A total of 27 guinea pigs (300-350 g by weight) were used for the immunization. The 27 guinea pigs were divided into 9 groups with 3 animals per group and an initial immunization with 400 μg of peptide followed by 2 boosters on 3, and 6 weeks post initial immunization (wpi), as shown in Table 15. Blood samples were collected at 0, 3, 6, 9, 12 and 15 wpi for titers of anti-IL-31 antibodies by its corresponding IL-31 B epitope peptide (SEQ ID NO:95). The nine groups are as shown in Table 15 were placebo (with PBS), CPG1, CpG3, ISA51VG, ADJUPHOS, ISA51VG+CpG1, ADJUPHOS+CpG1, ISA5VG+CPG3 and ADJUPHOS+CpG 3. Titration of serum anti-IL-31 antibodies was performed by ELISA using IL-31 B epitope Peptide (SEQ ID NO: 95) as described above where the titers are expressed in Log₁₀ as shown in Table 15 (for 0, 3, 6, 9, 12, and 15 wpi). The immunogenicity data were plotted in the upper panel of FIG. 24 for 0, 3, 6 and 9 wpi. Detailed average titers for each of the formulation groups were shown in Log₁₀ in the lower panel of FIG. 24.

Results from this immunogenicity study indicate the high potency of formulations using ISA51 and CpG as an water in oil emulsion performed better than those using ADJUPHOS as the adjuvant. Therefore the immunogenicity of the designer IL-31 peptide immunogen constructs can be further enhanced through optimal formulations.

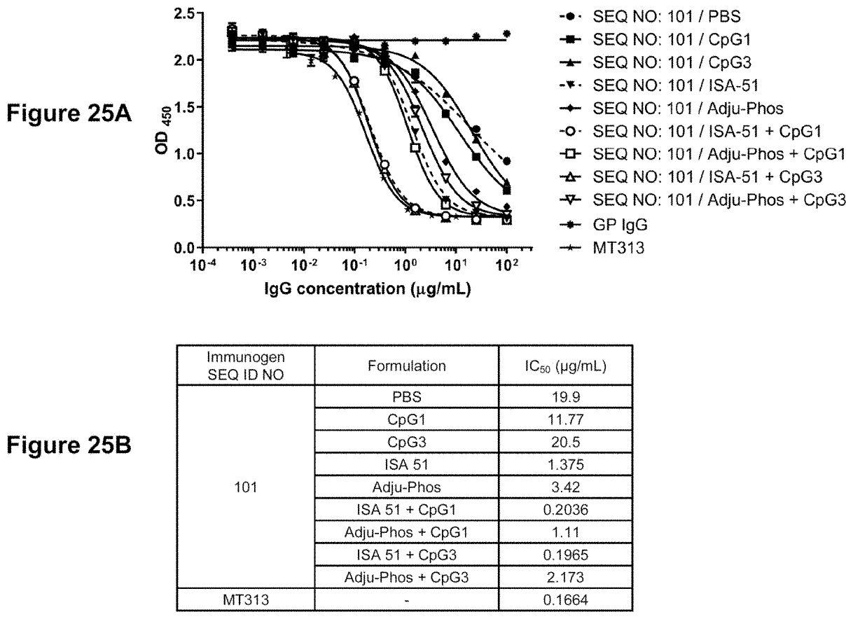

Purified polyclonal antibodies from these 9 wpi guinea pig immune sera against IL-31 peptide immunogen construct (SEQ ID NO:101) in various formulations were further tested in an IL-31 and IL-31Rα binding interaction assay as shown in FIG. 25. Detailed IC₅₀ (μg/mL) values for each of the formulations were calculated as shown in FIG. 25. Again, the most potent inhibitors of these antibodies are from those formulated in ISA51/CpG 1 or ISA51/CpG with IC₅₀ values being 0.2036 and 0.1965 μg/mL, respectively.

Furthermore, purified polyclonal antibodies from these 9 wpi guinea pig immune sera against IL-31 peptide immunogen construct (SEQ ID NO:101) in various formulations were tested in an ex-vivo mode for their ability to suppress IL-31-induced IL-20 expression and IL-31 induced STAT3 phosphorylation by these anti IL-31 antibodies.

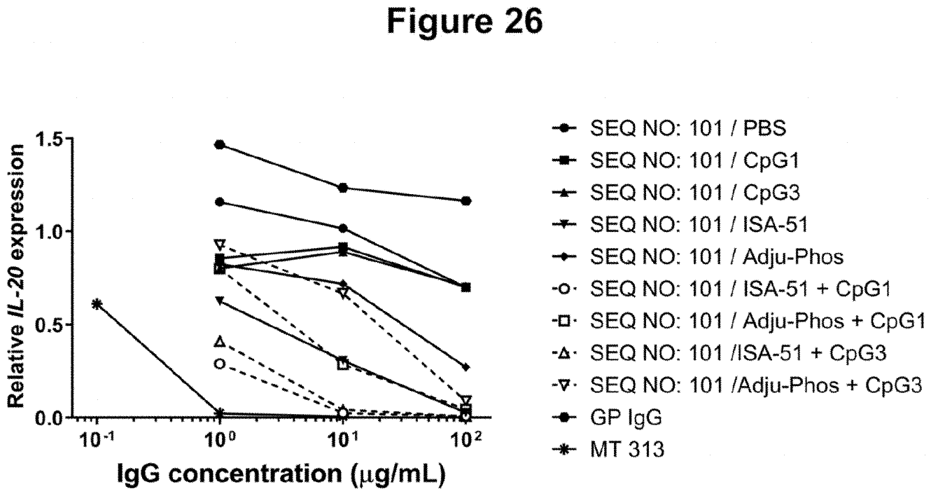

As shown in FIGS. 25 and 26, an antibody concentration-dependent suppression was observed in the treatment groups with purified antibodies elicited represented candidate peptide immunogen construct (SEQ ID NO: 101) in representative formulations (Groups 1 to 9). Again, formulations in ISA51+CPG1 or CpG3 as water in oil emulsions generated highest suppression of IL-20 expression as shown in FIG. 25 (from 0.3 or 0.4 fold of the control at 1 μg/mL down to zero when tested at 10 and 100 μg/mL) and similarly for STAT3 phosphorylation (FIG. 26).

### Example 12

### Further Assessment of Cross-Reactivities of Immunogenicity Across Different Species Amongst Canine, Murine and Human IL-31 Peptide Immunogen Constructs

a. IL-31 Based ELISA Test for Antibody Specificity Analysis

Individual canine, murine and human IL-31B epitope peptide (SEQ ID NOs: 12, 93, 96, 97 or 98) was immobilized on microtitre plates at 200 ng/well for respective IL-31 B epitope peptide in coating buffer (1.5 g/L Na₂CO₃, 2.9 g/L NaHCO₃, pH 9.6) and incubated at 4° C. overnight. Plates were washed 3 times with 200 μL/well of wash buffer (PBS with 0.05% TWEEN-20). Coated wells were blocked by 200 μL/well of assay diluent (0.5% BSA, 0.05% TWEEN-20, 0.01% ProClin 300 in PBS) at room temperature for 1 hour. Plates were washed 3 times with 200 μL/well of wash buffer, 100 μL of antiserum (serially 10-fold diluted) in antibody diluents were added to coated wells. The incubation was carried out at room temperature for 1 hour. All wells were aspirated and washed 5 times with 200 μL/well of wash buffer. The plates were incubated with a 1:10,000 dilution of HRP-conjugated goat anti-guinea pig IgG (H+L) antibody (Jackson ImmunoResearch Laboratories) for 1 hour (100 μL/well). Then all wells were washed 5 times with 200 μL/well of wash buffer. Finally, wells were developed by 100 μL/well of NeA-Blue TMB substrate (Clinical Science Products) and the reaction was stopped by addition of 100 μL/well of 1M H2SO4. The absorbance was measured at OD450 on an ELISA microplate reader (Molecule Devices). The specific titer of antiserum, expressed as Log₁₀ Titers was determined

b. Results

Results from the immunogenicity assessment study are shown in Tables 13 and 14.

Reasonable cross-reactivities with the IL-31 B epitope peptides (SEQ ID NO:12 and 93) were found amongst guinea pig immune sera against counterpart peptide analogues from both canine (SEQ ID NOs: 83 and 85) and human species (SEQ ID NO:99) as shown in Table 13. However, limited cross-reactivities for their binding to the corresponding IL-31 B epitope peptides (SEQ ID NOs: 96 for canine vs 97 and 98 for murine) were found for guinea pig immune sera directed against murine IL-31 peptide immunogen constructs (SEQ ID NOs: 104 and 105) with the counterpart peptide immunogen construct analogues from canine species (102 and 103). No cross-reactivities was found for guinea pig immune sera directed against canine IL-31 peptide immunogen constructs (SEQ ID NOs: 102 and 103) with their counterpart murine IL-31 B epitope peptide analogues (SEQ ID NOs:97 and 98) as shown in Table 14. Therefore, canine model can be used for proof of concept studies for human applications.

### Example 13

### Immunogenicity Assessment in Guinea Pigs of Recombinant Canine IL-31 Protein and a Lipoprotein Containing UBITH1 Linkage in Various Formulations with or without Adjuvant

Both recombinant full length canine IL-31 protein (FL-canine IL-31) and recombinant full length canine IL-31 protein (FL-canine IL-31 lipoprotein) comprising UBITh1 sequence placed at the N-terminal end of the IL-31 protein for immunogenicity enhancement were prepared in two types of formulations one without adjuvant and one with ADJUPHOS for comparison of their relative immunogenicities where the adjuvant free formulations were tested at two high and low dosings of 50 μg/0.5 mL/dose/IM and 25 μg/0.5 mL/dose/IM whereas the one with ADJUPHOS as adjuvant formulations were tested at high dosing only of 50 μg/0.5 mL/dose/IM with protocol shown in Table 12.

a. IL-31 Based ELISA Test for Antibody Specificity Analysis

Recombinant full length (FL) canine IL-31 protein was immobilized on microtitre plates at 50 ng/well in coating buffer (1.5 g/L Na₂CO₃, 2.9 g/L NaHCO3, pH 9.6) and incubated at 4° C. overnight. Plates were washed 3 times with 200 μL/well of wash buffer (PBS with 0.05% TWEEN-20). Coated wells were blocked by 200 μL/well of assay diluent (0.5% BSA, 0.05% TWEEN-20, 0.01% ProClin 300 in PBS) at room temperature for 1 hour. Plates were washed 3 times with 200 μL/well of wash buffer, 100 μL of antiserum (serially 10-fold diluted) in antibody diluents were added to coated wells. The incubation was carried out at room temperature for 1 hour. All wells were aspirated and washed 5 times with 200 μL/well of wash buffer. The plates were incubated with a 1:10,000 dilution of HRP-conjugated goat anti-guinea pig IgG (H+L) antibody (Jackson ImmunoResearch Laboratories) for 1 hour (100 μL/well). Then all wells were washed 5 times with 200 μL/well of wash buffer. Finally, wells were developed by 100 μL/well of NeA-Blue TMB substrate (Clinical Science Products) and the reaction was stopped by addition of 100 μL/well of 1M H2SO4. The absorbance was measured at OD450 on an ELISA microplate reader (Molecule Devices). The specific titer of antiserum, expressed as Log₁₀ Titers was determined

b. Results

Results from the immunogenicity assessment study of the above two recombinant proteins are shown in Table 12.

Both the canine IL-31 lipoprotein (Groups 1 to 3) and the regular canine IL-31 protein (Groups 4 to 6) were found immunogenic even in the absence of adjuvant (Groups 1, 2, 4, and 5) when such proteins were enhanced for their immunogenicity with UBITh® 1 linked at the N-terminal end. Overall, the immunogenicity of the IL-31 lipoprotein (groups 1 to 3) was found better than the regular IL-31 non-lipoprotein (groups 4 to 6) in parallel comparison of same formulations where those lipoprotein groups were found to be highly immunogenicity upon only single immunization. The regular protein immunization groups (groups 4 to 6) began to catch up with their corresponding immunogenicity upon further boosting and arrived at equivalent immunogenicity after two boosters as shown by immune sera bleeds of 8 wpi.

TABLE 1
Amino Acid Sequences of IL-31 and Fragments Thereof Employed in Serological Assays

| Amino Acid | SEQ ID | |
|---|---|---|
| positions | NO: | Sequence* |
| Canine IL-31 1-159 | 1 | MLSHT GPSRF ALFLL CSMET LLSSH MAPTH QLPPS DVRKI ILELQ PLSRG |
| (Uniprot C7G0W1-1; | | LLEDY QKKET GVPES NRTLL LCLTS DSQPP RLNSS AILPY FRAIR PLSDK |
| GenBank: BAH97742.1) | | NIIDK IIEQL DKLKF QHEPE TEISV PADTF ECKSF ILTIL QQFSA CLESV |
| | | FKSLN SGPQ |
| Human IL-31 1-164 | 2 | MASHS GPSTS VLFLF CCLGG WLASH TLPVR LLRPS DDVQK IVEEL QSLSK |
| (GenBank: AAS86448.1) | | MLLKD VEEEK GVLVS QNYTL PCLS DAQPP NNIHS PAIRA YLKTI RQLDN |
| | | KSVID EIIEH LDKLI FQDAP ETNIS VPTDT HECKR FILTI SQQFS ECMDL |
| | | ALKSL TSGAQ QATT |
| canine IL-31 97-144 | 3 | LSDKN IIDKI IEQLD KLKFQ HEPET EISVP ADTFE CKSFI LTILQ QFS |
| (P4751) | | |
| canine IL-31 97-133 | 4 | LSDKN IIDKI IEQLD KLKFQ HEPET EISVP ADTFE CK |
| (p4752) | | |
| canine IL-31 97-122 | 5 | LSDKN IIDKI IEQLD KLKFQ HEPET E |
| (p4753) | | |
| canine IL-31 97-114 | 6 | LSDKN IIDKI IEQLD KLK |
| (P4754) | | |
| canine IL-31 90-110 | 7 | YFRAI RPLSD KNIID KIIEQ L |
| (P4755) | | |
| canine IL-31 90-144 | 8 | YFRAI RPLSD KNIID KIIEQ LDKLK FQHEP ETEIS VPADT FECKS FILTI |
| (P4854)/(p4855) | | LQQFS |
| canine IL-31 86-144 | 9 | AILPY FRAIR PLSDK NIIDK IIEQL DKLKF QHEPE TEISV PADTF ECKSF |
| (P4856) | | ILTIL QQFS |
| canine IL-31 97-149 | 10 | LSDKN IIDKI IEQLD KLKFQ HEPET EISVP ADTFE CKSFI LTILQ QFSAS |
| (p4857) | | LES |
| canine IL-31 90-149 | 11 | YFRAI RPLSD KNIID KIIEQ LDKLK FQHEP ETEIS VPADT FECKS FILTI |
| (P4858) | | LQQFS ASLES |
| canine IL-31 86-149 | 12 | AILPY FRAIR PLSDK NIIDK IIEQL DKLKF QHEPE TEISV PADTF ECKSF |
| (P4859) | | ILTIL QQFSA SLES |
| human IL-31 98-145 | 13 | LDNKS VIDEI IEHLD KLIFQ DAPET NISVP TDTHE CKRFI LTISQ QFS |
| (P4847) | | |
| human IL-31 87-150 | 93 | AIRAY LKTIR QLDNK SVIDE IIEHL DKLIF QDAPE TNISV PTDTH ECKRF |
| (p5114) | | ILTIS QQFSE SMDL |
| human IL-31105-113 | 94 | DEIIE HLDK |
| (P5094) | | |
| human IL-31 85-115 | 95 | CPAIR AYLKT IRQLD NKSVI DEIIE HLDKL C |
| (P5095) | | |
| canine IL-31 84-114 | 96 | CSAIL PYFRA IRPLS DKNII DKIIE QLDKL C |
| (p5202)/(p5216) | | |
| murine IL-31 86-117 | 97 | CVIIA HLEKV KVLSE NTVDT SWVIR WLTNI SC |
| (p5217) | | |
| murine IL-31 87-116 | 98 | CIIAH LEKVK VLSEN TVDTS WVIRW LTNIC |
| (P5218) | | |

*The Amino acids replaced by Serine or Cysteine are underlined

TABLE 2
Selected Promiscuous T Helper Epitopes for Employment in the Design
of IL-31 Derived Peptide Immunogen Constructs

| | SEQ ID | Sequence |
|---|---|---|
| Description | NO: | |
| Clostridium tetani1 Th | 14 | KKQYIKANSKFIGITEL |
| MvF1 Th | 15 | LSEIKGVIVHRLEGV |
| Bordetella pertussis Th | 16 | GAYARCPNGTRALTVAELRGNAEL |
| Clostridium tetani2 Th | 17 | WVRDIIDDFTNESSQKT |
| Diphtheria Th | 18 | DSETADNLEKTVAALSILPGHGC |
| Plasmodium falciparum Th | 19 | DHEKKHAKMEKASSVFNVVNS |
| Schistosoma mansoni Th | 20 | KWFKTNAPNGVDEKHRH |
| Cholera Toxin Th | 21 | ALNIWDRFDVFCTLGATTGYLKGNS |
| MvF2 Th | 22 | ISEIKGVIVHKIEGI |
| KKKMvF3 Th | 23 | KKKISISEIKGVIVHKIEGILF |
| | | T RT TR T |
| HBsAg1 Th | 24 | KKKLFLLTKLLTLPQSLD |
| | | RRRIKII RII I L IR |
| | | VRVV VV V I V |
| | | F FF FF F V F |
| | | F |
| MvF4 Th (UBITh ®3) | 25 | ISISEIKGVIVHKIETILF |
| | | T RT TR |
| HBsAg2 Th | 26 | KKKIITITRIITIPQSLD |
| | | FFLL L ITTI |
| MvF5 Th (UBITh ®1) | 27 | ISITEIKGVIVHRIETILF |
| HBsAg3 Th (UBITh ®2) | 28 | KKKIITITRIITIITTID |
| Influenza MP1_1 Th | 29 | FVFTLTVPSER |
| Influenza MP1_2 Th | 30 | SGPLKAEIAQRLEDV |
| Influenza NSP1 Th | 31 | DRLRRDQKS |
| EBV BHRF1 Th | 32 | AGLTLSLLVICSYLFISRG |
| Clostridium tetani TT1 Th | 33 | QYIKANSKFIGITEL |
| EBV EBNA-1 Th | 34 | PGPLRESIVCYFMVFLQTHI |
| Clostridium tetani TT2 Th | 35 | FNNFTVSFWLRVPKVSASHLE |
| Clostridium tetani TT3 Th | 36 | KFIIKRYTPNNEIDSF |
| Clostridium tetani TT4 Th | 37 | VSIDKFRIFCKALN PK |
| EBV CP Th | 38 | VPGLYSPCRAFFNKEELL |
| HCMV IE1 Th | 39 | DKREMWMACIKELH |
| EBV GP340 Th | 40 | TGHGARTSTEPTTDY |
| EBV BPLF1 Th | 41 | KELKRQYEKKLRQ |
| EBV EBNA-2 Th | 42 | TVFYNIPPMPL |

TABLE 3
Amino Acid Sequences of IL-31 Peptide Immunogen Constructs

| Peptide | | SEQ | |
|---|---|---|---|
| code | Description | NO: | Sequence* |
| p4751kb | UBITh3-ϵK-KKK-canine IL-31₉₇₋₁₄₄ | 43 | UBITh3-ϵK-KKK LSDKNIIDKIIEQLDLKLFQHEPETEISVPADTFECKSFILTILQQFS |
| | UBITh3-ϵK-canine IL-31₉₇₋₁₄₄ | 44 | UBITh3-ϵK- LSDKNIIDKIIEQLDLKLFQHEPETEISVPADTFECKSFILTILQQFS |
| | UBITh1-ϵK-KKK-canine IL-31₉₇₋₁₄₄ | 45 | UBITh1-ϵK-KKK LSDKNIIDKIIEQLDLKLFQHEPETEISVPADTFECKSFILTILQQFS |
| | UBITh1-ϵK-canine IL-31₉₇₋₁₄₄ | 46 | UBITh1-ϵK- LSDKNIIDKIIEQLDLKLFQHEPETEISVPADTFECKSFILTILQQFS |
| p4752kb | UBITh3-ϵK-KKK-canine IL-31₉₇₋₁₃₃ | 47 | UBITh3-ϵK-KKK LSDKNIIDKIIEQLDKLKFQREPETEISVPADTFECK |
| | UBITh3-ϵK-canine IL-31₉₇₋₁₃₃ | 48 | UBITh3-ϵK- LSDKNIIDKIIEQLDKLKFQREPETEISVPADTFECK |
| | UBITh1-ϵK-KKK-canine IL-31₉₇₋₁₃₃ | 49 | UBITh1-ϵK-KKK LSDKNIIDKIIEQLDKLKFQREPETEISVPADTFECK |
| | UBITh1-ϵK-canine IL-31₉₇₋₁₃₃ | 50 | UBITh1-ϵK- LSDKNIIDKIIEQLDKLKFQREPETEISVPADTFECK |
| p4753kb | UBITh3-ϵK-KKK-canine IL-31₉₇₋₁₂₂ | 51 | UBITh3-ϵK-KKK LSDKNIIDKIIEQLDKLKFQHEPETE |
| | UBITh3-ϵK-canine IL-31₉₇₋₁₂₂ | 52 | UBITh3-ϵK- LSDKNIIDKIIEQLDKLKFQHEPETE |
| | UBITh1-ϵK-KKK-canine IL-31₉₇₋₁₂₂ | 53 | UBITh1-ϵK-KKK LSDKNIIDKIIEQLDKLKFQHEPETE |
| | UBITh1-ϵK-canine IL-31₉₇₋₁₂₂ | 54 | UBITh1-ϵK- LSDKNIIDKIIEQLDKLKFQHEPETE |
| p4754kb | UBITh3-ϵK-KKK-canine IL-31₉₇₋₁₁₄ | 55 | UBITh3-ϵK-KKK LSDKNIIDKIIEQLDKLK |
| | UBITh3-ϵK-canine IL-31₉₇₋₁₁₄ | 56 | UBITh3-ϵK- LSDKNIIDKIIEQLDKLK |
| | UBITh1-ϵK-KKK-canine IL-31₉₇₋₁₁₄ | 57 | UBITh1-ϵK-KKK LSDKNIIDKIIEQLDKLK |
| | UBITh1-ϵK-canine IL-31₉₇₋₁₁₄ | 58 | UBITh1-ϵK- LSDKNIIDKIIEQLDKLK |
| p4755kb | UBITh3-ϵK-KKK-canine IL-31₉₀₋₁₁₀ | 59 | UBITh3-ϵK-KKK YFRAIRPLSDKNIIDKIIEQL |
| | UBITh3-ϵK-canine IL-31₉₀₋₁₁₀ | 60 | UBITh3-ϵK- YFRAIRPLSDKNIIDKIIEQL |
| | UBITh1-ϵK-KKK-canine IL-31₉₀₋₁₁₀ | 61 | UBITh1-ϵK-KKK YFRAIRPLSDKNIIDKIIEQL |
| | UBITh1-ϵK-canine IL-31₉₀₋₁₁₀ | 62 | UBITh1-ϵK- YFRAIRPLSDKNIIDKIIEQL |
| p4854kb | UBITh3-ϵK-KKK-canine IL-31₉₀₋₁₄₄ | 63 | UBITh3-ϵK-KKK YFRAIRPLSDKNIIDKIIEQLDKLKFQHEPETEISVPADTFECKSFILTILQQFS |
| | UBITh3-ϵK-canine IL-31₉₀₋₁₄₄ | 64 | UBITh3-ϵK- YFRAIRPLSDKNIIDKIIEQLDKLKFQHEPETEISVPADTFECKSFILTILQQFS |
| | UBITh1-ϵK-KKK-canine IL-31₉₀₋₁₄₄ | 65 | UBITh1-ϵK-KKK YFRAIRPLSDKNIIDKIIEQLDKLKFQHEPETEISVPADTFECKSFILTILQQFS |
| | UBITh1-ϵK-canine IL-31₉₀₋₁₄₄ | 66 | UBITh1-ϵK- YFRAIRPLSDKNIIDKIIEQLDKLKFQHEPETEISVPADTFECKSFILTILQQFS |
| p4856kb | UBITh3-ϵK-KKK-canine IL-31₉₀₋₁₁₀ | 67 | UBITh3-ϵK-KKK YFRAIRPLSDKNIIDKIIEQLDKLKFQHEPETEISVPADTFECKSFILTILQQFS |
| | UBITh3-ϵK-canine IL-31₉₀₋₁₁₀ | 68 | UBITh3-ϵK- YFRAIRPLSDKNIIDKIIEQLDKLKFQHEPETEISVPADTFECKSFILTILQQFS |
| | UBITh1-ϵK-KKK-canine IL-31₉₀₋₁₁₀ | 69 | UBITh1-ϵK-KKK YFRAIRPLSDKNIIDKIIEQLDKLKFQHEPETEISVPADTFECKSFILTILQQFS |
| | UBITh1-ϵK-canine IL-31₉₀₋₁₁₀ | 70 | UBITh1-ϵK- YFRAIRPLSDKNIIDKIIEQLDKLKFQHEPETEISVPADTFECKSFILTILQQFS |
| p4856kb | UBITh3-ϵK-KKK-canine IL-31₈₆₋₁₄₄ | 71 | UBITh3-ϵK-KKK AILPYFRAIRPLSDKNIIDKIIEQLDKLKFQHEPETEISVPADTFECKSFILTILQQFS |
| | UBITh3-ϵK-canine IL-31₈₆₋₁₄₄ | 72 | UBITh3-ϵK- AILPYFRAIRPLSDKNIIDKIIEQLDKLKFQHEPETEISVPADTFECKSFILTILQQFS |
| | UBITh1-ϵK-KKK-canine IL-31₈₆₋₁₄₄ | 73 | UBITh1-ϵK-KKK AILPYFRAIRPLSDKNIIDKIIEQLDKLKFQHEPETEISVPADTFECKSFILTILQQFS |
| | UBITh1-ϵK-canine IL-31₈₆₋₁₄₄ | 74 | UBITh1-ϵK- AILPYFRAIRPLSDKNIIDKIIEQLDKLKFQHEPETEISVPADTFECKSFILTILQQFS |
| p4857kb | UBITh3-ϵK-KKK-canine IL-31₉₇₋₁₄₉ | 75 | UBITh3-ϵK-KKK LSDKNIIDKIIEQLDKLKFQHEPETEISVPADTFECKSFILTILQQFSASLES |
| | UBITh3-ϵK-canine IL-31₉₇₋₁₄₉ | 76 | UBITh3-ϵK- LSDKNIIDKIIEQLDKLKFQHEPETEISVPADTFECKSFILTILQQFSASLES |
| | UBITh1-ϵK-KKK-canine IL-31₉₇₋₁₄₉ | 77 | UBITh1-ϵK-KKK LSDKNIIDKIIEQLDKLKFQHEPETEISVPADTFECKSFILTILQQFSASLES |
| | UBITh1-ϵK-canine IL-31₉₇₋₁₄₉ | 78 | UBITh1-ϵK- LSDKNIIDKIIEQLDKLKFQHEPETEISVPADTFECKSFILTILQQFSASLES |
| p4858kb | UBITh3-ϵK-KKK-canine IL-31₈₆₋₁₄₄ | 79 | UBITh3-ϵK-KKK YFRAIRPLSDKNIIDKIIEQLDKLKFQHEPETEISVPADTFECKSFILTILQQFSASLES |
| | UBITh3-ϵK-canine IL-31₈₆₋₁₄₄ | 80 | UBITh3-ϵK- YFRAIRPLSDKNIIDKIIEQLDKLKFQHEPETEISVPADTFECKSFILTILQQFSASLES |
| | UBITh1-ϵK-KKK-canine IL-31₈₆₋₁₄₄ | 81 | UBITh1-ϵK-KKK YFRAIRPLSDKNIIDKIIEQLDKLKFQHEPETEISVPADTFECKSFILTILQQFSASLES |
| | UBITh1-ϵK-canine IL-31₈₆₋₁₄₄ | 82 | UBITh1-ϵK- YFRAIRPLSDKNIIDKIIEQLDKLKFQHEPETEISVPADTFECKSFILTILQQFSASLES |
| p5115kb | UBITh3-ϵK-KKK-canine IL-31₈₆₋₁₄₉ | 83 | UBITh3-ϵK-KKK AILPYFRAIRPLSDKNIIDKIIEQLDKLKFQHEPETEISVPADTFECKSFILTILQQFSASLES |
| p4859kb | UBITh3-ϵK-canine IL-31₈₆₋₁₄₉ | 84 | UBITh3-ϵK- AILPYFRAIRPLSDKNIIDKIIEQLDKLKFQHEPETEISVPADTFECKSFILTILQQFSASLES |
| p5114kb | UBITh1-ϵK-KKK-canine IL-31₈₆₋₁₄₉ | 85 | UBITh1-ϵK-KKK AILPYFRAIRPLSDKNIIDKIIEQLDKLKFQHEPETEISVPADTFECKSFILTILQQFSASLES |
| | UBITh1-ϵK-canine IL-31₈₆₋₁₄₉ | 86 | UBITh1-ϵK- AILPYFRAIRPLSDKNIIDKIIEQLDKLKFQHEPETEISVPADTFECKSFILTILQQFSASLES |
| p4847kb | UBITh1-ϵK-KKK-human IL-31₉₈₋₁₄₅ | 87 | UBITh1-ϵK-KKK LDNKSVIDEIIEHLDKLIFQDAPETNISVPTDTHECKRFILTISQQFS |
| | UBITh1-ϵK-human IL-31₉₈₋₁₄₅ | 88 | UBITh1-ϵK- LDNKSVIDEIIEHLDKLIFQDAPETNISVPTDTHECKRFILTISQQFS |
| | UBITh3-ϵK-KKK-human IL-31₉₈₋₁₄₅ | 89 | UBITh3-ϵK-KKK LDNKSVIDEIIEHLDKLIFQDAPETNISVPTDTHECKRFILTISQQFS |
| | UBITh3-ϵK-human IL-31₉₈₋₁₄₅ | 90 | UBITh3-ϵK- LDNKSVIDEIIEHLDKLIFQDAPETNISVPTDTHECKRFILTISQQFS |
| | spacer | 91 | -ϵK-KKK |
| | Flexible Hinge Spacer | 92 | PPXPXP |
| p5114kb | UBITh1-ϵK-KKK-human IL-31₈₇₋₁₆₀ | 99 | UBITh1-ϵK-KKK-AIRAYLKTIRQLDNKSVIDEIIEHLDKLIFQDAPETNISVPTDTHECKRFILTISQQFSESMDL |
| p5094kb | UBITh1-ϵK-KKK-human IL-31₁₀₅₋₁₁₃ | 100 | UBITh1-ϵK-KKK-DEIIEHLDK |
| p5095kb | UBITh1-ϵK-KKK-human IL-31₈₅₋₁₁₅ | 101 | UBITh1-ϵK-KKK-CPAIRAYLKTIRQLDNKSVIDEIIERLDKLC |
| p5202kb | UBITh1-ϵK-KKK-canine IL-31₈₄₋₁₁₄ | 102 | UBITh1-ϵK-KKK-CSAILPYFRAIRPLSDKNIIDKIIEQLDKLC |
| p5216kb | UBITh3-ϵK-KKK-canine IL-31₈₄₋₁₁₄ | 103 | UBITh3-ϵK-KKK-CSAILPYFRAIRPLSDKNIIDKIIEQLDKLC |
| p5217kb | UBITh3-ϵK-KKK-murine IL-31₈₆₋₁₁₇ | 104 | UBITh3-ϵK-KKK-CVIIAHLEKVKVLSENTVDTSWVIRWLTNISC |
| p5218kb | UBITh3-ϵK-KKK-murine IL-31₈₇₋₁₁₆ | 105 | UBITh3-ϵK-KKK-CIIAHLEKVKVLSENTVDTSWVIRWLTNIC |

*The Amino acids replaced by Serine or Cysteine are underlined; UBITh1 = SEQ ID NO: 27: UBITh3 = SEQ ID NO: 25

TABLE 4
Protocol for Assessment of Immunogenicity in Guinea
Pigs of Canine IL-31 Peptide Immunogen Constructs

| | | | # of | Immunogen dose/ | | | |
|---|---|---|---|---|---|---|---|
| | | SEQ | animals/ | volume/immunization | Formulation | Shot | Immunogen |
| Group# | Description | ID NO: | group ^{a} | site | and Adjuvant | No.^{b, c} | concentration |
| 1 | p4751kb | 43 | 3 | Prime: 400 μg/1.0 ml/ | ISA 50V2 | 5 | p4751Kb: |
| | UBITh3-εK- | | | dose/IM (four injection | peptide: CpG | | 400 ug/ml |
| | KKK-canine | | | sites 0.25 ml/site) | | | |
| | IL-31₉₇₋₁₄₄ | | | Boosts: 100 μg/0.25 | | | |
| | | | | ml/dose/IM | | | |
| 2 | p4752kb | 47 | 3 | Prime: 400 μg/1.0 ml/ | ISA 50V2 | 5 | p4752Kb: |
| | UBITh3-εK- | | | dose/IM (four injection | peptide: CpG | | 400 ug/ml |
| | KKK-canine | | | sites, 0.25 ml/site) | | | |
| | IL-31₉₇₋₁₃₃ | | | Boosts: 100 μg/0.25 | | | |
| | | | | ml/dose/IM | | | |
| 3 | p4753kb | 51 | 3 | Prime: 400 μg/1.0 ml/ | ISA 50V2 | 5 | p4753Kb: |
| | UBITh3-εK- | | | dose/IM (four injection | peptide: CpG | | 400 ug/ml |
| | KKK-canine | | | sites, 0.25 ml/site) | | | |
| | IL-31₉₇₋₁₂₂ | | | Boosts: 100 μg/0.25 | | | |
| | | | | ml/dose/IM | | | |
| 4 | p4754kb | 55 | 3 | Prime: 400 μg/1.0 ml/ | ISA 50V2 | 5 | p4754Kb: |
| | UBITh3-εK- | | | dose/IM (four injection | peptide: CpG | | 400 ug/ml |
| | KKK-canine | | | sites, 0.25 ml/site) | | | |
| | IL-31₉₇₋₁₁₄ | | | Boosts: 100 μg/0.25 | | | |
| | | | | ml/dose/IM | | | |
| 5 | p4755kb | 59 | 3 | Prime: 400 μg/1.0 ml/ | ISA 50V2 | 5 | p4755Kb: |
| | UBITh3-εK- | | | dose/IM (four injection | peptide: CpG | | 400 ug/ml |
| | KKK-canine | | | sites, 0.25 ml/site) | | | |
| | IL-31₉₀₋₁₁₀ | | | Boosts: 100 μg/0.25 | | | |
| | | | | ml/dose/IM | | | |

Immunizations administered at weeks 0, 3, 6, 9, and 12
Sera collected at weeks 0, 3, 6, 9, 12, and 15

TABLE 5
Immunogenicity Assessment in Guinea Pigs of Canine IL-31 Peptide Immunogen Constructs

| | | | | Canine IL-31₉₇₋₁₄₄ | | | | | | Canine IL-31₉₀₋₁₁₀ | | | | | |
|---|---|---|---|---|---|---|---|---|---|---|---|---|---|---|---|
| Group | | SEQ | Animal | (SEQ ID NO: 3) ELISA Log₁₀ Titer | | | | | | (SEQ ID NO: 7) ELISA Log₁₀ Titer | | | | | |
| # | Immunogen | ID NO: | No | 0 w | 3 w | 6 w | 9 w | 12 w | 15 w | 0 w | 3 w | 6 w | 9 w | 12 w | 15 w |
| 1 | p4751kb | 43 | 6045 | 0.157 | 5.867 | >10 | >10 | >10 | 10.05 | 0.141 | 0.000 | 3.243 | 3.527 | 4.168 | 4.607 |
| | UBITh3-εK- | | 6046 | 0.099 | 6.141 | >10 | 9.55 | 9.104 | 8.732 | 1.602 | 2.894 | 3.677 | 3.474 | 3.603 | 4.119 |
| | KKK-canine | | 6047 | 0.087 | 3.617 | >10 | >10 | >10 | >10 | 0.121 | 3.265 | 3.897 | 4.073 | 4.689 | 4.764 |
| | IL-31₉₇₋₁₄₄ | | | | | | | | | | | | | | |
| 2 | p4752kb | 47 | 6048 | 0.084 | 6.090 | 12.81 | 6.320 | 6.275 | 8.558 | 0.095 | 2.718 | 4.920 | 4.946 | 4.985 | 5.228 |
| | UBITh3-εK- | | 6049 | 0.121 | 5.181 | 8.393 | 7.149 | 7.713 | 8.787 | 0.092 | 0.000 | 2.735 | 3.254 | 4.124 | 4.666 |
| | KKK-canine | | 6050 | 0.098 | 6.364 | 6.904 | 5.848 | 5.824 | 6.026 | 0.105 | 3.252 | 3.778 | 3.870 | 4.234 | 4.674 |
| | IL-31₉₇₋₁₃₃ | | | | | | | | | | | | | | |
| 3 | p4753kb | 51 | 6051 | 0.088 | 9.561 | >10 | 8.099 | 7.406 | 6.638 | 0.107 | 3.185 | 4.734 | 4.287 | 4.023 | 4.308 |
| | UBITh3-εK- | | 6052 | 0.084 | 6.571 | >10 | >10 | >10 | 9.270 | 0.096 | 1.588 | 4.129 | 4.717 | 4.715 | 4.631 |
| | KKK-canine | | 6053 | 0.161 | 9.547 | 8.528 | 5.742 | 5.809 | 6.000 | 0.116 | 3.225 | 4 022 | 4.198 | 4.636 | 4.798 |
| | IL-31₉₇₋₁₂₂ | | | | | | | | | | | | | | |
| 4 | p4754kb | 55 | 6054 | 0.094 | 5.271 | 7.960 | 5.782 | 9.370 | 6.624 | 0.112 | 1.962 | 4.808 | 4.784 | 4.904 | 4.825 |
| | UBITh3-εK- | | 6055 | 0.107 | 7.918 | 8.744 | 5.983 | 6.341 | 6.077 | 0.121 | 3.150 | 4.258 | 4.284 | 4.321 | 4.345 |
| | KKK-canine | | 6056 | 0.068 | 8.406 | 11.79 | 7.093 | 9.391 | 10.34 | 0.091 | 3.286 | 4.781 | 4.955 | 5.019 | 5.680 |
| | IL-31₉₇₋₁₁₄ | | | | | | | | | | | | | | |
| 5 | p4755kb | 59 | 6057 | 0.113 | 4.169 | 4.753 | 4.738 | 4.877 | 4.703 | 0.091 | 4.951 | 6.153 | 5.302 | 5.347 | 5.137 |
| | UBITh3-εK- | | 6058 | 0.067 | 0.963 | 2.669 | 2.842 | 3.099 | 3.704 | 0.095 | 5.149 | 5.591 | 5.164 | 5.193 | 5.201 |
| | KKK-canine | | 6059 | 0.081 | 4.716 | 4.439 | 4.237 | 4.409 | 3.998 | 0.101 | 4.868 | 5.162 | 5.036 | 5.064 | 4.873 |
| | IL-31₉₀₋₁₁₀ | | | | | | | | | | | | | | |

TABLE 6
Antibody titer (Log₁₀ EC₅₀) to recombinant canine IL-31 protein

| | SEQ | | | | | |
|---|---|---|---|---|---|---|
| Immunogen | ID NO: | 3 wpi | 6 wpi | 9 wpi | 12 wpi | 15 wpi |
| p4751kb | 43 | 2.96 | 4.28 | 4.26 | 4.23 | 4.22 |
| P4752kb | 47 | 3.02 | 3.62 | 3.5 | 3.77 | 3.94 |
| p4753kb | 51 | 2.82 | 3.7 | 3.44 | 3.25 | 2.82 |
| p4754kb | 55 | 2.89 | 3.03 | 3.23 | 3.15 | 3.38 |
| p4755kb | 59 | 2.97 | 2.89 | 2.95 | 2.5 | 2.63 |

TABLE 7
Protocol for Assessment of Immunogenicity in Guinea Pigs of Canine IL-31 Peptide Immunogen Constructs

| | | | # of | | | | |
|---|---|---|---|---|---|---|---|
| | | SEQ | animals/ | Immunogen dose/volume/ | Formulation | Shot | Immunogen |
| Group# | Description | ID NO: | group ^{a} | immunization site | and Adjuvant | No.^{b, c} | concentration |
| 1 | p4854kb | 63 | 3 | Prime: 400 μg/1.0 ml/ | ISA 50V2 | 5 | p4854Kb: |
| | UBITh3-εK- | | | dose/IM (four injection | peptide: CpG | | 400 ug/ml |
| | KKK-Canine | | | sites, 0.25 ml/site) | | | |
| | IL31₉₀₋₁₄₄ | | | Boosts: 100 μg/0.25 | | | |
| | | | | ml/dose/IM | | | |
| 2 | p4855kb | 68 | 3 | Prime: 400 μg/1.0 ml/ | ISA 50V2 | 5 | p4855Kb: |
| | UBITh3-εK- | | | dose/IM (four injection | peptide: CpG | | 400 ug/ml: |
| | Canine IL31₉₀₋₁₄₄ | | | sites, 0.25 ml/site) | | | |
| | | | | Boosts: 100 μg/0.25 | | | |
| | | | | ml/dose/IM | | | |
| 3 | p4856kb | 71 | 3 | Prime 400 μg/1.0 ml/ | ISA 50V2 | 5 | p4856Kb: |
| | UBITh3-εK- | | | dose/IM (four injection | peptide: CpG | | 400 ug/ml |
| | KKK-Canine | | | sites, 0.25 ml/site) | | | |
| | IL31 ₈₀₋₁₄₄ | | | Boosts: 100 μg/0.25 | | | |
| | | | | ml/dose/IM | | | |
| 4 | p4857kb | 76 | 3 | Prime: 400 μg/1.0 ml/ | ISA 50V2 | 5 | p4857Kb: |
| | UBITh3-εK- | | | dose/IM (four injection | peptide: CpG | | 400 ug/ml: |
| | Canine IL31₉₇₋₁₄₉ | | | sites, 0.25 ml/site) | | | |
| | | | | Boosts: 100 μg/0.25 | | | |
| | | | | ml/dose/IM | | | |
| 5 | p4858kb | 80 | 3 | Prime: 400 μg/1.0 ml/ | ISA 50V2 | 5 | p4858Kb: |
| | UBITh3-εK- | | | dose/IM (four injection | peptide: CpG | | 400 ug/ml |
| | Canine IL31₈₀₋₁₄₉ | | | sites, 0.25 ml/site) | | | |
| | | | | Boosts: 100 μg/0.25 | | | |
| | | | | ml/dose/IM | | | |
| 6 | p4859kb | 84 | 3 | Prime: 400 μg/1.0 ml/ | ISA 50V2 | 5 | p4859Kb: |
| | UBITh3-εK- | | | dose/IM (four injection | peptide: CpG | | 400 ug/ml |
| | Canine IL31₈₆₋₁₄₉ | | | sites, 0.25 ml/site) | | | |
| | | | | Boosts: 100 μg/0.25 | | | |
| | | | | ml/dose/IM | | | |

Immunizations administered at weeks 0, 3, 6, 9, and 12
Sera collected at weeks 0, 3, 6, 9, 12, and 15

TABLE 8
Immunogenicity Assessment in Guinea Pigs of Canine IL-31 Peptide Immunogen Constructs

| | | | | Canine IL-31₈₆₋₁₄₄ | | | | | | Canine IL-31₈₆₋₁₄₉ | | | | | |
|---|---|---|---|---|---|---|---|---|---|---|---|---|---|---|---|
| Group | | SEQ | Animal | (SEQ ID NO: 9) ELISA Log₁₀ Titer | | | | | | (SEQ ID NO: 12) ELISA Log₁₀ Titer | | | | | |
| # | Immunogen | ID NO: | No | 0 w | 3 w | 6 w | 9 w | 12 w | 15 w | 0 w | 3 w | 6 w | 9 w | 12 w | 15 w |
| 1 | p4854kb | 63 | 6264 | 0.19 | 7.69 | >10 | 11.7 | >10 | >10 | 0.20 | >10 | >10 | >10 | >10 | >10 |
| | UBITh3-εK- | | 6265 | 0.14 | 5.41 | >10 | 11.8 | >10 | >10 | 0.14 | 6.67 | >10 | >10 | >10 | >10 |
| | KKK-canine | | 6266 | 0.14 | 9.31 | >10 | 9.3 | >10 | >10 | 0.14 | >10 | >10 | >10 | >10 | >10 |
| | IL-31₉₇₋₁₄₄ | | | | | | | | | | | | | | |
| 2 | p4855kb | 68 | 6267 | 0.14 | 7.37 | >10 | 10.8 | >10 | 9.04 | 0.13 | 12.99 | >10 | >10 | >10 | >10 |
| | UBITh3-εK- | | 6268 | 0.15 | 9.12 | >10 | 10.5 | 11.9 | >10 | 0.16 | 15.37 | >10 | >10 | 11.6 | 11.40 |
| | KKK-canine | | 6269 | 0.16 | 5.46 | 7.06 | 7.1 | 6.1 | 6.22 | 0.14 | 5.99 | 7.54 | 9.05 | 6.1 | 6.71 |
| | IL-31₉₇₋₁₃₃ | | | | | | | | | | | | | | |
| 3 | p4856kb | 71 | 6270 | 0.19 | 5.97 | >10 | 10.8 | >10 | 9.42 | 0.13 | 7.98 | >10 | >10 | >10 | >10 |
| | UBITh3-εK- | | 6271 | 0.19 | 6.30 | >10 | 9.2 | 9.8 | 9.44 | 0.21 | 8.83 | >10 | >10 | 9.6 | >10 |
| | KKK-canine | | 6272 | 0.21 | 5.17 | >10 | 9.6 | >10 | >10 | 0.15 | 5.72 | >10 | >10 | >10 | >10 |
| | IL-31₉₇₋₁₂₂ | | | | | | | | | | | | | | |
| 4 | p4857kb | 76 | 6273 | 0.11 | 6.64 | >10 | >10 | >10 | 9.14 | 0.13 | 8.85 | >10 | >10 | >10 | 10.93 |
| | UBITh3-εK- | | 6274 | 0.13 | 5.63 | >10 | 7.5 | 6.7 | 5.86 | 0.13 | 6.74 | >10 | 8.58 | 6.8 | 6.14 |
| | KKK-canine | | 6275 | 0.10 | 5.33 | >10 | >10 | >10 | >10 | 0.09 | 6.28 | >10 | >10 | >10 | >10 |
| | IL-31₉₇₋₁₁₄ | | | | | | | | | | | | | | |
| 5 | p4858kb | 80 | 6276 | 0.11 | 8.27 | >10 | >10 | >10 | >10 | 0.10 | 11.54 | >10 | >10 | >10 | >10 |
| | UBITh3-εK- | | 6277 | 0.12 | 6.66 | >10 | >10 | >10 | >10 | 0.14 | 8.36 | >10 | >10 | >10 | >10 |
| | KKK-canine | | 6278 | 0.14 | 6 64 | >10 | 10.6 | 11.0 | 8.21 | 0.11 | 8.65 | >10 | >10 | 10.2 | 10.33 |
| | IL-31₉₀₋₁₁₀ | | | | | | | | | | | | | | |
| 6 | p4859kb | 84 | 6279 | 0.14 | 5.88 | >10 | >10 | 11.1 | 8.58 | 0.14 | 7.19 | >10 | >10 | 10.8 | 10.16 |
| | UBITh3-εK- | | 6280 | 0.18 | 8.46 | >10 | 10.6 | 10.8 | 8.55 | 0.19 | 11.02 | >10 | >10 | 10.5 | 10.89 |
| | KKK-canine | | 6281 | 0.09 | 6.37 | >10 | >10 | >10 | >10 | 0.11 | 7.39 | >10 | >10 | >10 | >10 |
| | IL-31₉₀₋₁₁₀ | | | | | | | | | | | | | | |

TABLE 9
Immunogenicity Assessment in Guinea Pigs of Canine
IL-31 Peptide Immunogen Constructs for their cross-
reactivities with recombinant canine IL-31 protein

| Immunogen | SEQ ID NO | 3 wpi | 6 wpi | 9 wpi | 12 wpi |
|---|---|---|---|---|---|
| p4854kb | 63 | 3.12 | 4.1 | 5.51 | 5.53 |
| P4855kb | 68 | 3.3 | 4.29 | 5.38 | 5.12 |
| p4856kb | 71 | 2.88 | 4.06 | 5.08 | 5.14 |
| p4857kb | 76 | 3.13 | 4.35 | 5.32 | 4.71 |
| p4858kb | 80 | 3.14 | 4.5 | 5.41 | 5.18 |
| p4859kb | 84 | 3.21 | 4.47 | 4.94 | 4.72 |

TABLE 10
Immunogenicity Assessment in Guinea Pigs of
Human IL-31 Peptide Immunogen Constructs

| | | | | Human IL-31₈₉₋₁₄₅ | | | | | |
|---|---|---|---|---|---|---|---|---|---|
| Group | | SEQ | Animal | (SEQ ID NO: 13) ELISA Log₁₀ Titer | | | | | |
| # | Immunogen | ID NO: | No | 0 w | 3 w | 6 w | 9 w | 12 w | 15 w |
| 1 | p4847kb | 87 | 6222 | 0.133 | 10.326 | >10 | >10 | >10 | >10 |
| | UBITh1-εK- | | 6223 | 0.110 | >10 | 9.483 | >10 | 9.576 | >10 |
| | KKK-human | | 6224 | 0.108 | >10 | >10 | >10 | >10 | >10 |
| | IL-31₉₈₋₁₄₅ | | | | | | | | |

TABLE 11
Immunogenicity Assessment in Guinea Pigs of Human IL-31 Peptide Immunogen Constructs

| | | | | Human IL-31₁₀₅₋₁₁₃ | | | | | | Human IL-31₈₅₋₁₁₅ | | | | | |
|---|---|---|---|---|---|---|---|---|---|---|---|---|---|---|---|
| Group | | SEQ | Animal | (SEQ ID NO: 94) ELISA Log₁₀ Titer | | | | | | (SEQ ID NO: 95) ELISA Log₁₀ Titer | | | | | |
| # | Immunogen | ID NO: | No | 0 w | 3 w | 6 w | 9 w | 12 w | 15 w | 0 w | 3 w | 6 w | 9 w | 12 w | 15 w |
| 1 | p5094kb | 100 | 6664 | 0.131 | 4.229 | 5.245 | 5.340 | 5.298 | 5.086 | 0.097 | 3.430 | 4.853 | 4.931 | 4.923 | 4.775 |
| | UBITh1-εK- | | 6665 | 0.069 | 5.060 | 5.557 | 5.526 | 5.340 | 5.155 | 0.073 | 5.126 | 5.529 | 5.395 | 5.060 | 4.868 |
| | KKK-human | | 6666 | 0.079 | 4.814 | 5.658 | 7.622 | 5.401 | 5.177 | 0.084 | 4.713 | 5.100 | 5.082 | 4.861 | 4.736 |
| | IL-31₁₀₅₋₁₁₃ | | | | | | | | | | | | | | |
| 2 | p5095kb | 101 | 6667 | 0.072 | 1.499 | 4.193 | 3.445 | 3.532 | 3.361 | 0.181 | 5.683 | >10 | >10 | 7.738 | 6.322 |
| | UBITh1-εK- | | 6668 | 0.066 | 0.000 | 2.843 | 3.604 | 3.719 | 3.973 | 0.080 | 6.168 | >10 | >10 | 12.71 | 9.136 |
| | KKK-human | | 6669 | 0.059 | 0.000 | 3.473 | 3.446 | 3.565 | 3.473 | 0.070 | 5.793 | >10 | >10 | 7.044 | 5.989 |
| | IL-31₈₅₋₁₁₅ | | | | | | | | | | | | | | |

TABLE 12
Immunogenicity Assessment in Guinea Pigs of Full Length Recombinant
Human IL-31 Protein and its Lipoprotein Counterpart

| Group | | | | Animal | Anti-recombinant canine IL31 titers | | | | | | |
|---|---|---|---|---|---|---|---|---|---|---|---|
| # | Immunogen | Dose | Adjuvant | No | 0 wpi | 3 wpi | 6 wpi | 8 wpi | 10 wpi | 12 wpi | 14 wpi |
| 1 | FL- canine | Prime & | 20 mM Tris, | 6796 | 0.112 | 2.723 | 4.823 | 5.060 | 5.016 | 4.805 | 4.658 |
| | IL31 | boosts: | 140 mM NaCl, | 6797 | 0.071 | 3.096 | 4.697 | 4.911 | 4.763 | 4.613 | 3.987 |
| | lipoprotein | 50 μg/0.5 | 0.02% tween 80 | 6798 | 0.082 | 3.368 | 4.715 | 4.883 | 4.726 | 4.421 | 3.601 |
| | | ml/dose/IM | | | | | | | | | |
| 2 | FL- canine | Prime & | | 6799 | 0.070 | 3.573 | 5.012 | 5.241 | 5.145 | 5.034 | 4.848 |
| | IL31 | boosts; | | 6800 | 0.069 | 2.660 | 4.367 | 4.611 | 4.387 | 3.674 | 3.501 |
| | lipoprotein | 25 μg/0.5 | | 6801 | 0.060 | 3.635 | 4.801 | 4.843 | 4.850 | 4.764 | 4.762 |
| | | ml/dose/IM | | | | | | | | | |
| 3 | FL- canine | Prime & | 20 mM Tris, | 6802 | 0.067 | 4.086 | 4.967 | 5.052 | 4.929 | 4.719 | 4.581 |
| | IL31 | boosts: | 140 mM NaCl, | 6803 | 0.063 | 3.396 | 4.766 | 4.733 | 4.508 | 4.331 | 4.257 |
| | lipoprotein | 25 μg/0.5 | 0.02% tween 80 + | 6804 | 0.066 | 3.211 | 4.911 | 5.005 | 4.869 | 4.781 | 4.734 |
| | | ml/dose/IM | Adju-Phos | | | | | | | | |
| 4 | FL- canine | Prime & | 20 mM Tris, | 6805 | 0.065 | 2.133 | 4.522 | 5.018 | 4.875 | 4.767 | 4.723 |
| | IL31 protein | boosts: | 140 mM NaCl, | 6806 | 0.072 | 0.190 | 4.031 | 4.948 | 4.814 | 4.798 | 4.744 |
| | | 50 μg/0.5 | 0.02% tween 80 | 6807 | 0.062 | 2.776 | 4.769 | 5,163 | 5.034 | 4.904 | 4.920 |
| | | ml/dose/IM | | | | | | | | | |
| 5 | FL- canine | Prime & | | 6808 | 0.057 | 2.497 | 4.840 | 5.240 | 5.049 | 4.972 | 4.860 |
| | IL31 protein | boosts: | | 6809 | 0.058 | 2.730 | 4.869 | 5.247 | 5.023 | 4.924 | 4.882 |
| | | 25 μg/0.5 | | 6810 | 0.060 | 0.000 | 3.069 | 5.027 | 4.935 | 4.900 | 4.851 |
| | | ml/dose/IM | | | | | | | | | |
| 6 | FL- canine | Prime & | 20 mM Tris, | 6811 | 0.060 | 0.000 | 3.537 | 5.214 | 5.032 | 4.902 | 4.830 |
| | IL31 protein | boosts: | 140 mM NaCl, | 6812 | 0.069 | 0.000 | 2.767 | 4.861 | 4.716 | 4.690 | 4.688 |
| | | 25 μg/0.5 | 0.02% tween 80 + | 6813 | 0.071 | 0.000 | 3.481 | 4.735 | 4.578 | 4.169 | 3.654 |
| | | ml/dose/IM | Adju-Phos | | | | | | | | |

Immunizations administered at weeks 0, 3 and 6
Sera collected at weeks 0, 3, 6, 8, 10, 12 and 14

TABLE 13
Immunogenicity Assessment in Guinea Pigs of canine and human IL-31 Peptide
Immunogen Constructs and for Their Respective Cross-reactivities

| | | | | | canine IL-31₈₆₋₁₄₉ | | | | | Human IL-31₈₇₋₁₅₀ | | | | |
|---|---|---|---|---|---|---|---|---|---|---|---|---|---|---|
| Group | | | SEQ | Animal | (SEQ ID NO: 12) ELISA Log₁₀ Titer | | | | | (SEQ ID NO: 93) ELISA Log₁₀ Titer | | | | |
| # | Immunogen | Adjuvant | ID NO: | No | 0 w | 3 w | 6 w | 9 w | 12 w | 0 w | 3 w | 6 w | 9 w | 12 w |
| 1 | p5113kb: | ISA 50V2 | 85 | 6814 | 0.082 | >10 | >10 | >10 | >10 | 0.094 | 3.182 | 4.834 | 4.451 | 4.720 |
| | UBITh1-εK- | CpG3 | | 6815 | 0.107 | >10 | >10 | >10 | >10 | 0.117 | 2.620 | 3.926 | 3.211 | 3.378 |
| | KKK-canine | Tween 80 | | 6816 | 0.078 | >10 | >10 | >10 | >10 | 0.088 | 4.679 | 4.000 | 3.330 | 3.270 |
| | IL31₈₆₋₁₄₉ | | | | | | | | | | | | | |
| 2 | p5115kb: | | 83 | 6817 | 0.080 | >10 | >10 | >10 | >10 | 0.113 | 3.320 | 5.042 | 4.965 | 5.009 |
| | UBITh3-εK- | | | 6818 | 0.075 | >10 | >10 | >10 | >10 | 0.084 | 0.703 | 3.528 | 3.438 | 4.700 |
| | KKK-canine | | | 6819 | 0.078 | >10 | >10 | >10 | >10 | 0.096 | 4.324 | 6.392 | 6.693 | 6.669 |
| | IL31₈₆₋₁₄₉ | | | | | | | | | | | | | |
| 3 | p5114kb: | ISA 51VG | 99 | 6820 | 0.078 | 3.586 | 3.285 | 3.235 | 3.084 | 0.082 | >10 | >10 | >10 | >10 |
| | UBITh1-εK- | CpG3 | | 6821 | 0.083 | 4.303 | 4.878 | 4.414 | 3.590 | 0.086 | >10 | >10 | >10 | >10 |
| | KKK-human | Tween 80 | | 6822 | 0.081 | 4.606 | 6.532 | 5.367 | 5.425 | 0.092 | >10 | >10 | >10 | >10 |
| | IL31₈₇₋₁₅₀ | | | | | | | | | | | | | |

TABLE 14
Immunogenicity Assessment in Guinea Pigs of Canine and Murine IL-31 Peptide
Immunogen Constructs and for Their Respective Cross-reactivities

| | | | | | | | | | | Murine IL-31₈₆₋₁₁₇ | |
|---|---|---|---|---|---|---|---|---|---|---|---|
| | | | | Canine IL-31₈₄₋₁₁₄ | | | | | | (SEQ ID NO: 97) | |
| Group | | SEQ | Animal | (SEQ ID NO: 96) ELISA Log₁₀ Titer | | | | | | ELISA Log₁₀ Titer | |
| # | Immunogen | ID NO: | No | 0 w | 3 w | 6 w | 9 w | 12 w | 15 w | 0 w | 3 w |
| 1 | p5202kb | 102 | 7126 | 0.141 | 4.962 | 5.201 | 5.276 | 6.002 | 5.886 | 0.084 | 0.000 |
| | UBITh1-εK- | | 7127 | 0.077 | 5.165 | 5.750 | 5.767 | 6.417 | 6.714 | 0.070 | 0.000 |
| | KKK-Canine | | 7128 | 0.084 | 5.063 | 5.233 | 5.276 | 5.835 | 5.694 | 0.080 | 0.000 |
| | IL-31₈₄₋₁₁₄ | | | | | | | | | | |
| 2 | p5216kb | 103 | 7129 | 0.097 | 4.937 | 5.300 | 5.383 | 6.379 | 8.201 | 0.088 | 0.000 |
| | UBITh3-εK- | | 7130 | 0.115 | 5.124 | 6.072 | 6.260 | 6.472 | 5.404 | 0.087 | 0.000 |
| | KKK-Canine | | 7131 | 0.079 | 5.191 | 7.780 | 6.126 | 6.191 | 5.519 | 0.080 | 0.000 |
| | IL-31₈₄₋₁₁₄ | | | | | | | | | | |
| 3 | p5217kb | 104 | 7132 | 0.106 | 2.293 | 4.084 | 4.189 | 3.997 | 3.894 | 0.073 | 1.840 |
| | UBITh3-εK- | | 7133 | 0.135 | 3.017 | 4.236 | 4.631 | 4.777 | 4.530 | 0.070 | 3.134 |
| | KKK-Murine | | 7134 | 0.079 | 0.000 | 4.633 | 4.895 | 5.019 | 4.850 | 0.075 | 1.708 |
| | IL-31₈₆₋₁₁₇ | | | | | | | | | | |
| 4 | p5218kb | 105 | 7135 | 0.080 | 0.000 | 2.232 | 0.958 | 2.895 | 2.337 | 0.080 | 2.814 |
| | UBITh3-εK- | | 7136 | 0.077 | 0.000 | 1.845 | 0.000 | 2.734 | 3.039 | 0.080 | 0.000 |
| | KKK-Murine | | 7137 | 0.087 | 1.531 | 4.310 | 3.649 | 3.386 | 3.156 | 0.084 | 0.000 |
| | IL-31₈₇₋₁₁₆ | | | | | | | | | | |

| | | Murine IL-31₈₆₋₁₁₇ | | | | | | | | | |
|---|---|---|---|---|---|---|---|---|---|---|---|
| | | (SEQ ID NO: 97) | | | | Murine IL-31₈₇₋₁₁₆ | | | | | |
| Group | | ELISA Log₁₀ Titer | | | | (SEQ ID NO: 98) ELISA Log₁₀ Titer | | | | | |
| # | Immunogen | 6 w | 9 w | 12 w | 15 w | 0 w | 3 w | 6 w | 9 w | 12 w | 15 w |
| 1 | p5202kb | 0.000 | 0.000 | 0.000 | 0.000 | 0.086 | 0.000 | 0.000 | 0.000 | 0.000 | 0.000 |
| | UBITh1-εK- | 0.000 | 0.000 | 0.578 | 0.000 | 0.085 | 0.000 | 0.000 | 0.000 | 0.625 | 0.000 |
| | KKK-Canine | 0.000 | 0.000 | 0.304 | 0.000 | 0.094 | 0.000 | 0.000 | 0.000 | 0.567 | 0.533 |
| | IL-31₈₄₋₁₁₄ | | | | | | | | | | |
| 2 | p5216kb | 0.000 | 0.000 | 0.065 | 0.000 | 0.142 | 0.000 | 0.000 | 0.000 | 0.013 | 0.000 |
| | UBITh3-εK- | 0.000 | 0.000 | 0.908 | 0.000 | 0.080 | 0.000 | 0.000 | 0.000 | 0.000 | 0.000 |
| | KKK-Canine | 0.000 | 0.000 | 2.414 | 0.000 | 0.075 | 0.000 | 0.000 | 0.000 | 1.383 | 0.314 |
| | IL-31₈₄₋₁₁₄ | | | | | | | | | | |
| 3 | p5217kb | 4.987 | 5.174 | 5.318 | 5.091 | 0.070 | 1.450 | 4.935 | 5.063 | 5.123 | 4.994 |
| | UBITh3-εK- | 5.172 | 5.259 | 5.263 | 5.208 | 0.075 | 3.136 | 5.143 | 5.144 | 5.112 | 5.064 |
| | KKK-Murine | 4.749 | 4.913 | 4.989 | 4.838 | 0.077 | 0.384 | 4.669 | 4.791 | 4.836 | 4.690 |
| | IL-31₈₆₋₁₁₇ | | | | | | | | | | |
| 4 | p5218kb | 6.315 | 6.093 | 5.827 | 5.299 | 0.091 | 2.766 | 6.612 | 5.933 | 5.540 | 5.214 |
| | UBITh3-εK- | 5.091 | 5.127 | 5.187 | 5.058 | 0.087 | 0.000 | 5.083 | 5.101 | 5.081 | 4.924 |
| | KKK-Murine | 5.312 | 5.760 | 5.738 | 5.422 | 0.099 | 0.000 | 5.198 | 5.892 | 5.366 | 5.155 |
| | IL-31₈₇₋₁₁₆ | | | | | | | | | | |

TABLE 15
Immunogenicity Assessment of a Representative human IL-31 Peptide Immunogen
Construct (SEQ ID NO: 101) in Guinea Pigs in Multi-Component Formulations

| | | | | Human IL-31₈₅₋₁₁₅ | | | | | |
|---|---|---|---|---|---|---|---|---|---|
| Group | | | Animal | (SEQ ID NO: 95) ELISA Log₁₀ Titer | | | | | |
| # | Immunogen | Adjuvant | No | 0 w | 3 w | 6 w | 9 w | 12 w | 15 w |
| 1 | p5095kb | — | 6982 | 0.072 | 2.827 | 3.793 | 4.728 | 4.494 | 3.782 |
| | UBITh1-εK- | | 6983 | 0.080 | 1.563 | 4.384 | 4.728 | 4.678 | n/a |
| | KKK-human | | 6984 | 0.077 | 0.000 | 3.149 | 4.510 | 4.399 | 3.838 |
| 2 | IL-31₈₅₋₁₁₅ | CpG1 | 6985 | 0.072 | 0.000 | 4.223 | 4.954 | 4.834 | 4.739 |
| | SEO ID NO: | | 6986 | 0.064 | 0.000 | 3.689 | 4.076 | 3.441 | 3.295 |
| | 101 | | 6987 | 0.064 | 0.000 | 1.273 | 4.252 | 3.962 | 3.400 |
| 3 | | CpG3 | 6988 | 0.077 | 1.926 | 3.788 | 4.469 | 4.147 | 3.605 |
| | | | 6989 | 0.068 | 3.186 | 4.792 | 5.001 | 4.826 | 4.744 |
| | | | 6990 | 0.064 | 0.000 | 2.487 | 3.970 | 3.336 | 3.047 |
| 4 | | ISA 51VG | 6991 | 0.132 | 4.506 | 4.803 | 4.939 | 4.847 | 4.806 |
| | | | 6992 | 0.085 | 3.702 | 4.648 | 4.745 | 4.708 | 4.369 |
| | | | 6993 | 0.092 | 4.055 | 4.874 | 4.942 | 4.956 | 5.073 |
| 5 | | Adju-Phos | 6994 | 0.068 | 1.917 | 3.460 | 4.760 | 4.603 | 3.815 |
| | | | 6995 | 0.071 | 2.583 | 4.614 | 4.925 | 4.772 | 4.517 |
| | | | 6996 | 0.073 | 2.986 | 4.072 | 4.304 | 3.801 | 3.308 |
| 6 | | ISA 51VG + | 6997 | 0.066 | 4.915 | 5.454 | 6.046 | 5.498 | 5.230 |
| | | CpG1 | 6998 | 0.064 | 4.734 | 5.381 | 6.567 | 5.851 | 5.399 |
| | | | 6999 | 0.071 | 4.863 | 5.228 | 6.288 | 5.760 | 5.228 |
| 7 | | Adju-Phos + | 7000 | 0.083 | 0.672 | 4.486 | 4.783 | 4.691 | 4.608 |
| | | CpG1 | 7001 | 0.067 | 2.570 | 4.255 | 4.730 | 4.499 | 4.084 |
| | | | 7002 | 0.073 | 3.791 | 5.010 | 5.200 | 5.056 | 4.956 |
| 8 | | ISA 51VG + | 7003 | 0.066 | 5.061 | 6.060 | 7.033 | 5.731 | 5.204 |
| | | CpG3 | 7004 | 0.063 | 4.476 | 4.974 | 5.305 | 5.075 | 4.990 |
| | | | 7005 | 0.065 | 3.076 | 5.163 | 5.588 | 5.558 | 5.086 |
| 9 | | Adju-Phos + | 7006 | 0.064 | 3.072 | 4.549 | 4.789 | 4.985 | 4.703 |
| | | CpG3 | 7007 | 0.070 | 3.071 | 4.808 | 5.010 | 5.153 | 4.909 |
| | | | 7008 | 0.090 | 3.041 | 4.708 | 4.949 | 4.947 | 4.789 |

1. An IL-31 peptide immunogen construct represented by the formulae:
(Th)ₘ-(A)ₙ-(IL-31 fragment)-X
or
(IL-31 fragment)-(A)ₙ-(Th)ₘ-X
wherein
Th is a heterologous T helper epitope;
A is a heterologous spacer;
(IL-31 fragment) is a B cell epitope having about 15 to about 75 amino acid residues from SEQ ID NO: 1 or SEQ ID NO: 2;
X is an α-COOH or α-CONH₂ of an amino acid;
m is from 1 to about 4; and
n is from 0 to about 10.

2. The IL-31 peptide immunogen construct according to claim 1, wherein the IL-31 fragment is selected from the group consisting of SEQ ID NOs: 1-13 and 93-98.

3. The IL-31 peptide immunogen construct according to claim 1, wherein the Th epitope is selected from the group consisting of SEQ ID NOs: 14-42.

4. The IL-31 peptide immunogen construct according to claim 1, wherein the peptide immunogen construct is selected from the group consisting of SEQ ID NOs: 43-90 and 99-105.

5. An IL-31 peptide immunogen construct comprising:
a B cell epitope comprising about 15 to about 75 amino acid residues from the full-length IL-31 protein sequence of SEQ ID NO: 1 or SEQ ID NO: 2;
a T helper epitope comprising an amino acid sequence selected from the group consisting of SEQ ID NOs: 14-42; and
an optional heterologous spacer selected from the group consisting of an amino acid, Lys-, Gly-, Lys-Lys-Lys-, (α, ε-N)Lys, and ε-N-Lys-Lys-Lys-Lys (SEQ ID NO: 91),
wherein the B cell epitope is covalently linked to the T helper epitope directly or through the optional heterologous spacer.

6. The IL-31 peptide immunogen construct of claim 5, wherein the B cell epitope is selected from the group consisting of SEQ ID NOs: 1-13 and 93-98.

7. The IL-31 peptide immunogen construct of claim 5, wherein the T helper epitope is selected from the group consisting of SEQ ID NOs: 14-42.

8. The IL-31 peptide immunogen construct of claim 5, wherein the optional heterologous spacer is (α, ε-N)Lys or ε-N-Lys-Lys-Lys-Lys (SEQ ID NO: 91).

9. The IL-31 peptide immunogen construct of claim 5, wherein the T helper epitope is covalently linked to the amino terminus of the B cell epitope.

10. The IL-31 peptide immunogen construct of claim 5, wherein the T helper epitope is covalently linked to the amino terminus of the B cell epitope through the optional heterologous spacer.

11. A composition comprising a peptide immunogen construction according to claim 1.

12. A pharmaceutical composition comprising:
a. a peptide immunogen construct according to claim 1; and
b. and a pharmaceutically acceptable delivery vehicle and/or adjuvant.

13. The pharmaceutical composition of claim 12, wherein
a. the IL-31 peptide immunogen construct is selected from the group consisting of SEQ ID NOs: 43-90 and 99-105; and
b. the IL-31 peptide immunogen construct is mixed with an CpG oligodeoxynucleotide (ODN) to form a stabilized immunostimulatory complex.

14. An isolated antibody or epitope-binding fragment thereof that specifically binds to the B cell epitope of the IL-31 peptide immunogen construct according to claim 1.

15. The isolated antibody or epitope-binding fragment thereof according to claim 14 bound to the IL-31 peptide immunogen construct.

16. A composition comprising the isolated antibody or epitope-binding fragment thereof according to claim 14.

17. A pharmaceutical composition comprising the isolated antibody or epitope-binding fragment thereof according to claim 14.


SEQUENCE LISTING (110 sequences)

SEQ ID NO: 1 - protein, 159 aa, Canis lupus
  PEPTIDE (1)..(159): Canine IL-31 1-159 (Uniprot C7G0W1-1; GenBank BAH97742.1)
MLSHTGPSRFALFLLCSMETLLSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 2 - protein, 164 aa, Homo sapiens
  PEPTIDE (1)..(164): Human IL-31 1-164 (GenBank AAS86448.1)
MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 3 - protein, 48 aa, Canis lupus
  PEPTIDE (1)..(48): canine IL-31 97-144
LSDKNIIDKIIEQLDKLKFQHEPETEISVPADTFECKSFILTILQQFS

SEQ ID NO: 4 - protein, 37 aa, Canis lupus
  PEPTIDE (1)..(37): canine IL-31 97-133
LSDKNIIDKIIEQLDKLKFQHEPETEISVPADTFECK

SEQ ID NO: 5 - protein, 26 aa, Canis lupus
  PEPTIDE (1)..(26): canine IL-31 97-122
LSDKNIIDKIIEQLDKLKFQHEPETE

SEQ ID NO: 6 - protein, 18 aa, Canis lupus
  PEPTIDE (1)..(18): canine IL-31 97-114
LSDKNIIDKIIEQLDKLK

SEQ ID NO: 7 - protein, 21 aa, Canis lupus
  PEPTIDE (1)..(21): canine IL-31 90-110
YFRAIRPLSDKNIIDKIIEQL

SEQ ID NO: 8 - protein, 55 aa, Canis lupus
  PEPTIDE (1)..(55): canine IL-31 90-144
YFRAIRPLSDKNIIDKIIEQLDKLKFQHEPETEISVPADTFECKSFILTILQQFS

SEQ ID NO: 9 - protein, 59 aa, Canis lupus
  PEPTIDE (1)..(59): canine IL-31 86-144
AILPYFRAIRPLSDKNIIDKIIEQLDKLKFQHEPETEISVPADTFECKSFILTILQQFS

SEQ ID NO: 10 - protein, 53 aa, Canis lupus
  PEPTIDE (1)..(53): canine IL-31 97-149, C146 to S146
LSDKNIIDKIIEQLDKLKFQHEPETEISVPADTFECKSFILTILQQFSASLES

SEQ ID NO: 11 - protein, 60 aa, Canis lupus
  PEPTIDE (1)..(60): canine IL-31 90-149, C146 to S146
YFRAIRPLSDKNIIDKIIEQLDKLKFQHEPETEISVPADTFECKSFILTILQQFSASLES

SEQ ID NO: 12 - protein, 64 aa, Canis lupus
  PEPTIDE (1)..(64): canine IL-31 86-149, C146 to S146
AILPYFRAIRPLSDKNIIDKIIEQLDKLKFQHEPETEISVPADTFECKSFILTILQQFSASLES

SEQ ID NO: 13 - protein, 48 aa, Homo sapiens
  PEPTIDE (1)..(48): human IL-31 98-145
LDNKSVIDEIIEHLDKLIFQDAPETNISVPTDTHECKRFILTISQQFS

SEQ ID NO: 14 - protein, 17 aa, Clostridium tetani
  PEPTIDE (1)..(17): Clostridium tetani 1 Th
KKQYIKANSKFIGITEL

SEQ ID NO: 15 - protein, 15 aa, Measles virus
  PEPTIDE (1)..(15): MvF1 Th
LSEIKGVIVHRLEGV

SEQ ID NO: 16 - protein, 24 aa, Bordetella pertussis
  PEPTIDE (1)..(24): Bordetella pertussis Th
GAYARCPNGTRALTVAELRGNAEL

SEQ ID NO: 17 - protein, 17 aa, Clostridium tetani
  PEPTIDE (1)..(17): Clostridium tetani 2 Th
WVRDIIDDFTNESSQKT

SEQ ID NO: 18 - protein, 23 aa, diphtheria bacilli
  PEPTIDE (1)..(23): Diphtheria Th
DSETADNLEKTVAALSILPGHGC

SEQ ID NO: 19 - protein, 21 aa, Plasmodium falciparum
  PEPTIDE (1)..(21): Plasmodium falciparum Th
DHEKKHAKMEKASSVFNVVNS

SEQ ID NO: 20 - protein, 17 aa, Schistosoma mansoni
  PEPTIDE (1)..(17): Schistosoma mansoni Th
KWFKTNAPNGVDEKHRH

SEQ ID NO: 21 - protein, 25 aa, Cholera Toxin
  PEPTIDE (1)..(25): Cholera Toxin Th
ALNIWDRFDVFCTLGATTGYLKGNS

SEQ ID NO: 22 - protein, 15 aa, Measles virus
  PEPTIDE (1)..(15): MvF 2 Th
ISEIKGVIVHKIEGI

SEQ ID NO: 23 - protein, 22 aa, Measles virus
  PEPTIDE (1)..(22): KKKMvF 3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
KKKISIXEIXXVIVXXIEXILF

SEQ ID NO: 24 - protein, 18 aa, Hepatitis B virus
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
XXXXXXXTXXXTXPXSXX

SEQ ID NO: 25 - protein, 19 aa, Measles virus
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
ISIXEIXXVIVXXIETILF

SEQ ID NO: 26 - protein, 18 aa, Hepatitis B virus
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
KKKXXXXTRIXTIXXXXD

SEQ ID NO: 27 - protein, 19 aa, Measles virus
  PEPTIDE (1)..(19): MvF 5 Th
ISITEIKGVIVHRIETILF

SEQ ID NO: 28 - protein, 18 aa, Hepatitis B virus
  PEPTIDE (1)..(18): HBsAg 3 Th
KKKIITITRIITIITTID

SEQ ID NO: 29 - protein, 11 aa, Influenza virus
  PEPTIDE (1)..(11): Influenza Matrix protein 1 _1 Th
  PEPTIDE (1)..(11): Influenza Matrix protein 1_1 Th
FVFTLTVPSER

SEQ ID NO: 30 - protein, 15 aa, Influenza virus
  PEPTIDE (1)..(15): Influenza Matrix protein 1_2 Th
SGPLKAEIAQRLEDV

SEQ ID NO: 31 - protein, 9 aa, Influenza virus
  PEPTIDE (1)..(9): Influenza Non-structural protein 1 Th
DRLRRDQKS

SEQ ID NO: 32 - protein, 19 aa, Epstein-Barr virus
  PEPTIDE (1)..(19): EBV BHRF1 Th
AGLTLSLLVICSYLFISRG

SEQ ID NO: 33 - protein, 15 aa, Clostridium tetani
  PEPTIDE (1)..(15): Clostridium tetani TT1 Th
QYIKANSKFIGITEL

SEQ ID NO: 34 - protein, 20 aa, Epstein-Barr virus
  PEPTIDE (1)..(20): EBV EBNA-1 Th
PGPLRESIVCYFMVFLQTHI

SEQ ID NO: 35 - protein, 21 aa, Clostridium tetani
  PEPTIDE (1)..(21): Clostridium tetani  TT2 Th
FNNFTVSFWLRVPKVSASHLE

SEQ ID NO: 36 - protein, 16 aa, Clostridium tetani
  PEPTIDE (1)..(16): Clostridium tetani TT3 Th
KFIIKRYTPNNEIDSF

SEQ ID NO: 37 - protein, 16 aa, Clostridium tetani
  PEPTIDE (1)..(16): Clostridium tetani  TT4 Th
VSIDKFRIFCKALNPK

SEQ ID NO: 38 - protein, 18 aa, Epstein-Barr virus
  PEPTIDE (1)..(18): EBV CP Th
VPGLYSPCRAFFNKEELL

SEQ ID NO: 39 - protein, 14 aa, Human cytomegalovirus
  PEPTIDE (1)..(14): HCMV IE1 Th
DKREMWMACIKELH

SEQ ID NO: 40 - protein, 15 aa, Epstein-Barr virus
  PEPTIDE (1)..(15): EBV GP340 Th
TGHGARTSTEPTTDY

SEQ ID NO: 41 - protein, 13 aa, Epstein-Barr virus
  PEPTIDE (1)..(13): EBV BPLF1 Th
KELKRQYEKKLRQ

SEQ ID NO: 42 - protein, 11 aa, Epstein-Barr virus
  PEPTIDE (1)..(11): EBV EBNA-2 Th
TVFYNIPPMPL

SEQ ID NO: 43 - protein, 71 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(71): canine IL-31 97-144
ISIXEIXXVIVXXIETILFKKKKLSDKNIIDKIIEQLDKLKFQHEPETEISVPADTFECKSFILTILQQFS

SEQ ID NO: 44 - protein, 68 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(68): canine IL-31 97-144
ISIXEIXXVIVXXIETILFKLSDKNIIDKIIEQLDKLKFQHEPETEISVPADTFECKSFILTILQQFS

SEQ ID NO: 45 - protein, 71 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(71): canine IL-31 97-144
ISITEIKGVIVHRIETILFKKKKLSDKNIIDKIIEQLDKLKFQHEPETEISVPADTFECKSFILTILQQFS

SEQ ID NO: 46 - protein, 68 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(68): canine IL-31 97-144
ISISEIKGVIVHKIETILFKLSDKNIIDKIIEQLDKLKFQHEPETEISVPADTFECKSFILTILQQFS

SEQ ID NO: 47 - protein, 60 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(60): canine IL-31 97-133
ISIXEIXXVIVXXIETILFKKKKLSDKNIIDKIIEQLDKLKFQHEPETEISVPADTFECK

SEQ ID NO: 48 - protein, 57 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(57): canine IL-31 97-133
ISIXEIXXVIVXXIETILFKLSDKNIIDKIIEQLDKLKFQHEPETEISVPADTFECK

SEQ ID NO: 49 - protein, 60 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(60): canine IL-31 97-133
ISITEIKGVIVHRIETILFKKKKLSDKNIIDKIIEQLDKLKFQHEPETEISVPADTFECK

SEQ ID NO: 50 - protein, 57 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(57): canine IL-31 97-133
ISISEIKGVIVHKIETILFKLSDKNIIDKIIEQLDKLKFQHEPETEISVPADTFECK

SEQ ID NO: 51 - protein, 49 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(49): canine IL-31 97-122
ISIXEIXXVIVXXIETILFKKKKLSDKNIIDKIIEQLDKLKFQHEPETE

SEQ ID NO: 52 - protein, 46 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(46): canine IL-31 97-122
ISIXEIXXVIVXXIETILFKLSDKNIIDKIIEQLDKLKFQHEPETE

SEQ ID NO: 53 - protein, 49 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(49): canine IL-31 97-122
ISITEIKGVIVHRIETILFKKKKLSDKNIIDKIIEQLDKLKFQHEPETE

SEQ ID NO: 54 - protein, 46 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(46): canine IL-31 97-122
ISISEIKGVIVHKIETILFKLSDKNIIDKIIEQLDKLKFQHEPETE

SEQ ID NO: 55 - protein, 41 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(41): canine IL-31 97-114
ISIXEIXXVIVXXIETILFKKKKLSDKNIIDKIIEQLDKLK

SEQ ID NO: 56 - protein, 38 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(38): canine IL-31 97-114
ISIXEIXXVIVXXIETILFKLSDKNIIDKIIEQLDKLK

SEQ ID NO: 57 - protein, 41 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(41): canine IL-31 97-114
ISITEIKGVIVHRIETILFKKKKLSDKNIIDKIIEQLDKLK

SEQ ID NO: 58 - protein, 38 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(38): canine IL-31 97-114
ISISEIKGVIVHKIETILFKLSDKNIIDKIIEQLDKLK

SEQ ID NO: 59 - protein, 44 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(44): canine IL-31 90-110
ISIXEIXXVIVXXIETILFKKKKYFRAIRPLSDKNIIDKIIEQL

SEQ ID NO: 60 - protein, 41 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(41): canine IL-31 90-110
ISIXEIXXVIVXXIETILFKYFRAIRPLSDKNIIDKIIEQL

SEQ ID NO: 61 - protein, 44 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(44): canine IL-31 90-110
ISITEIKGVIVHRIETILFKKKKYFRAIRPLSDKNIIDKIIEQL

SEQ ID NO: 62 - protein, 41 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(41): canine IL-31 90-110
ISISEIKGVIVHKIETILFKYFRAIRPLSDKNIIDKIIEQL

SEQ ID NO: 63 - protein, 78 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(78): canine IL-31 90-144
ISIXEIXXVIVXXIETILFKKKKYFRAIRPLSDKNIIDKIIEQLDKLKFQHEPETEISVPADTFECKSFILTILQQFS

SEQ ID NO: 64 - protein, 75 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(75): canine IL-31 90-144
ISIXEIXXVIVXXIETILFKYFRAIRPLSDKNIIDKIIEQLDKLKFQHEPETEISVPADTFECKSFILTILQQFS

SEQ ID NO: 65 - protein, 78 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(78): canine IL-31 90-144
ISITEIKGVIVHRIETILFKKKKYFRAIRPLSDKNIIDKIIEQLDKLKFQHEPETEISVPADTFECKSFILTILQQFS

SEQ ID NO: 66 - protein, 75 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(75): canine IL-31 90-144
ISISEIKGVIVHKIETILFKYFRAIRPLSDKNIIDKIIEQLDKLKFQHEPETEISVPADTFECKSFILTILQQFS

SEQ ID NO: 67 - protein, 78 aa, Artificial Sequence; identical to SEQ ID NO: 63
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(78): canine IL-31 90-144

SEQ ID NO: 68 - protein, 75 aa, Artificial Sequence; identical to SEQ ID NO: 64
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(75): canine IL-31 90-144

SEQ ID NO: 69 - protein, 78 aa, Artificial Sequence; identical to SEQ ID NO: 65
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(78): canine IL-31 90-144

SEQ ID NO: 70 - protein, 75 aa, Artificial Sequence; identical to SEQ ID NO: 66
  Synthetic peptide
  PEPTIDE (1)..(19): MvF 5 Th (UBITh1)
  SITE (20)..(20): epsilon-K as a spacer
  PEPTIDE (21)..(75): canine IL-31 90-144

SEQ ID NO: 71 - protein, 82 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(82): canine IL-31 86-144
ISIXEIXXVIVXXIETILFKKKKAILPYFRAIRPLSDKNIIDKIIEQLDKLKFQHEPETEISVPADTFECKSFILTILQQFS

SEQ ID NO: 72 - protein, 79 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(79): canine IL-31 86-144
ISIXEIXXVIVXXIETILFKAILPYFRAIRPLSDKNIIDKIIEQLDKLKFQHEPETEISVPADTFECKSFILTILQQFS

SEQ ID NO: 73 - protein, 82 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(82): canine IL-31 86-144
ISITEIKGVIVHRIETILFKKKKAILPYFRAIRPLSDKNIIDKIIEQLDKLKFQHEPETEISVPADTFECKSFILTILQQFS

SEQ ID NO: 74 - protein, 79 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(79): canine IL-31 86-144
ISISEIKGVIVHKIETILFKAILPYFRAIRPLSDKNIIDKIIEQLDKLKFQHEPETEISVPADTFECKSFILTILQQFS

SEQ ID NO: 75 - protein, 76 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(76): canine IL-31 97-149, C146 to S146
ISIXEIXXVIVXXIETILFKKKKLSDKNIIDKIIEQLDKLKFQHEPETEISVPADTFECKSFILTILQQFSASLES

SEQ ID NO: 76 - protein, 73 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(73): canine IL-31 97-149, C146 to S146
ISIXEIXXVIVXXIETILFKLSDKNIIDKIIEQLDKLKFQHEPETEISVPADTFECKSFILTILQQFSASLES

SEQ ID NO: 77 - protein, 76 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(76): canine IL-31 97-149, C146 to S146
ISITEIKGVIVHRIETILFKKKKLSDKNIIDKIIEQLDKLKFQHEPETEISVPADTFECKSFILTILQQFSASLES

SEQ ID NO: 78 - protein, 73 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(73): canine IL-31 97-149, C146 to S146
ISISEIKGVIVHKIETILFKLSDKNIIDKIIEQLDKLKFQHEPETEISVPADTFECKSFILTILQQFSASLES

SEQ ID NO: 79 - protein, 83 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(83): canine IL-31 90-149, C146 to S146
ISIXEIXXVIVXXIETILFKKKKYFRAIRPLSDKNIIDKIIEQLDKLKFQHEPETEISVPADTFECKSFILTILQQFSASLES

SEQ ID NO: 80 - protein, 80 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(80): canine IL-31 90-149, C146 to S146
ISIXEIXXVIVXXIETILFKYFRAIRPLSDKNIIDKIIEQLDKLKFQHEPETEISVPADTFECKSFILTILQQFSASLES

SEQ ID NO: 81 - protein, 83 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(83): canine IL-31 90-149, C146 to S146
ISITEIKGVIVHRIETILFKKKKYFRAIRPLSDKNIIDKIIEQLDKLKFQHEPETEISVPADTFECKSFILTILQQFSASLES

SEQ ID NO: 82 - protein, 80 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(80): canine IL-31 90-149, C146 to S146
ISISEIKGVIVHKIETILFKYFRAIRPLSDKNIIDKIIEQLDKLKFQHEPETEISVPADTFECKSFILTILQQFSASLES

SEQ ID NO: 83 - protein, 87 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(87): canine IL-31 86-149, C146 to S146
ISIXEIXXVIVXXIETILFKKKKAILPYFRAIRPLSDKNIIDKIIEQLDKLKFQHEPETEISVPADTFECKSFILTILQQFSASLES

SEQ ID NO: 84 - protein, 84 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(84): canine IL-31 86-149, C146 to S146
ISIXEIXXVIVXXIETILFKAILPYFRAIRPLSDKNIIDKIIEQLDKLKFQHEPETEISVPADTFECKSFILTILQQFSASLES

SEQ ID NO: 85 - protein, 87 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(87): canine IL-31 86-149, C146 to S146
ISITEIKGVIVHRIETILFKKKKAILPYFRAIRPLSDKNIIDKIIEQLDKLKFQHEPETEISVPADTFECKSFILTILQQFSASLES

SEQ ID NO: 86 - protein, 84 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(84): canine IL-31 86-149, C146 to S146
ISISEIKGVIVHKIETILFKAILPYFRAIRPLSDKNIIDKIIEQLDKLKFQHEPETEISVPADTFECKSFILTILQQFSASLES

SEQ ID NO: 87 - protein, 71 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(71): human IL-31 98-145
ISITEIKGVIVHRIETILFKKKKLDNKSVIDEIIEHLDKLIFQDAPETNISVPTDTHECKRFILTISQQFS

SEQ ID NO: 88 - protein, 68 aa, Artificial Sequence
  Synthetic peptide
  PEPTIDE (1)..(19): MvF 5 Th (UBITh1)
  SITE (20)..(20): epsilon-K as a spacer
  PEPTIDE (21)..(68): human IL-31 98-145
ISISEIKGVIVHKIETILFKLDNKSVIDEIIEHLDKLIFQDAPETNISVPTDTHECKRFILTISQQFS

SEQ ID NO: 89 - protein, 71 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(71): human IL-31 98-145
ISIXEIXXVIVXXIETILFKKKKLDNKSVIDEIIEHLDKLIFQDAPETNISVPTDTHECKRFILTISQQFS

SEQ ID NO: 90 - protein, 68 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K as a spacer
  PEPTIDE (21)..(68): human IL-31 98-145
ISIXEIXXVIVXXIETILFKLDNKSVIDEIIEHLDKLIFQDAPETNISVPTDTHECKRFILTISQQFS

SEQ ID NO: 91 - protein, 4 aa, Artificial Sequence
  Synthetic peptide
  SITE (1)..(1): epsilon-K
  PEPTIDE (1)..(4): epsilon-K-KKK as a spacer
KKKK

SEQ ID NO: 92 - protein, 6 aa, Artificial Sequence
  Synthetic peptide
  PEPTIDE (1)..(6): Flexible Hinge Spacer
PPXPXP

SEQ ID NO: 93 - protein, 64 aa, Homo sapiens
  PEPTIDE (1)..(64): human IL-31 87-150, C147 to S147
AIRAYLKTIRQLDNKSVIDEIIEHLDKLIFQDAPETNISVPTDTHECKRFILTISQQFSESMDL

SEQ ID NO: 94 - protein, 9 aa, Homo sapiens
  PEPTIDE (1)..(9): human IL-31 105-113
DEIIEHLDK

SEQ ID NO: 95 - protein, 31 aa, Homo sapiens
  PEPTIDE (1)..(31): human IL-31 85-115, S85 to C85, I115 to C115
CPAIRAYLKTIRQLDNKSVIDEIIEHLDKLC

SEQ ID NO: 96 - protein, 31 aa, Canis lupus
  PEPTIDE (1)..(31): canine IL-31 84-114, S84 to C84, K114 to C114
CSAILPYFRAIRPLSDKNIIDKIIEQLDKLC

SEQ ID NO: 97 - protein, 32 aa, Mus musculus
  1PEPTIDE (1)..(32): murine IL-31 86-117, S86 to C86
CVIIAHLEKVKVLSENTVDTSWVIRWLTNISC

SEQ ID NO: 98 - protein, 30 aa, Mus musculus
  PEPTIDE (1)..(30): murine IL-31 87-116, V87 to C87,  S116 to C116
CIIAHLEKVKVLSENTVDTSWVIRWLTNIC

SEQ ID NO: 99 - protein, 87 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(87): human IL-31 87-150, C147 to S147
ISITEIKGVIVHRIETILFKKKKAIRAYLKTIRQLDNKSVIDEIIEHLDKLIFQDAPETNISVPTDTHECKRFILTISQQFSESMDL

SEQ ID NO: 100 - protein, 32 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(32): human IL-31 105-113
ISITEIKGVIVHRIETILFKKKKDEIIEHLDK

SEQ ID NO: 101 - protein, 54 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(54): human IL-31 85-115, S85 to C85, I115 to C115
ISITEIKGVIVHRIETILFKKKKCPAIRAYLKTIRQLDNKSVIDEIIEHLDKLC

SEQ ID NO: 102 - protein, 54 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(54): canine IL-31 84-114, S84 to C84, K114 to C114
ISITEIKGVIVHRIETILFKKKKCSAILPYFRAIRPLSDKNIIDKIIEQLDKLC

SEQ ID NO: 103 - protein, 54 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(54): canine IL-31 84-114, S84 to C84, K114 to C114
ISIXEIXXVIVXXIETILFKKKKCSAILPYFRAIRPLSDKNIIDKIIEQLDKLC

SEQ ID NO: 104 - protein, 55 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(55): murine IL-31 86-117, S86 to C86
ISIXEIXXVIVXXIETILFKKKKCVIIAHLEKVKVLSENTVDTSWVIRWLTNISC

SEQ ID NO: 105 - protein, 53 aa, Artificial Sequence
  Synthetic peptide
  PEPTIDE (1)..(19): MvF 4 Th (UBITH3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(53): murine IL-31 87-116, V87 to C87,  S116 to C116
ISIXEIXXVIVXXIETILFKKKKCIIAHLEKVKVLSENTVDTSWVIRWLTNIC

SEQ ID NO: 106 - DNA, 21 bp, Artificial Sequence
  HPRT forward primer
tgacactggcaaaacaatgca

SEQ ID NO: 107 - DNA, 21 bp, Artificial Sequence
  HPRT Reverse Primer
ggtccttttcaccagcaagct

SEQ ID NO: 108 - DNA, 32 bp, Artificial Sequence
  CpG1 oligonucleotide ODN
tcgtcgttttgtcgttttgtcgttttgtcgtt

SEQ ID NO: 109 - DNA, 24 bp, Artificial Sequence
  CpG2 oligonucleotide ODN
  misc_feature (1)..(1): phosphorothioate group
tcgtcgttttgtcgttttgtcgtt

SEQ ID NO: 110 - DNA, 24 bp, Artificial Sequence
  CpG3 Oligonucleotide ODN
tcgtcgttttgtcgttttgtcgtt